(19) United States
(12) Patent Application Publication
(10) Pub. No.: US 2004/0209363 A1
(43) Pub. Date: Oct. 21, 2004

(54) Methods and compositions for modulating T cell activation and uses thereof

(76) Inventors: Tania Watts, Toronto (CA); Jacob Bukczynski, Toronto (CA); Tao Wen, Saint-Laurent (CA)

Correspondence Address: KENYON & KENYON, ONE BROADWAY, NEW YORK, NY, 10004, US

(21) Appl. No.: 10/756,783

(22) Filed: Jan. 12, 2004

Related U.S. Application Data
(63) Continuation-in-part of application No. PCT/CA02/01033, filed on Jul. 5, 2002.

(51) Int. Cl. C12N 5/08; C12N 15/861

(52) U.S. Cl. 435/455; 435/456; 435/372

(57) ABSTRACT

The invention relates to methods and products for the preparation of antigen presenting cells, and for the modulation of T cell activation, for example to prepare actvated T cells. The invention further relates to antigen presenting cells and activated T cells thus prepared. The invention further relates to methods and uses of such antigen presenting cells for activation of T cells in vitro, ex vivo, or in vivo, for treatment of e.g. a condition associated with immuno impairment, or for vaccination. The invention further relates to methods and uses of such activated T cells for treatment of e.g. a condition associated with immuno impairment.

### CROSS-REFERENCE TO RELATED APPLICATIONS

This application is a continuation in part of PCT International Patent Application No. PCT/CA02/01033 filed Jul. 5, 2002, which was published in English under PCT Article 21(2), and which claims the benefit under Title 35, United States Code, § 119(e), of U.S. Provisional Patent Application Serial No. 60/304,430 filed Jul. 12, 2001. All of these applications are incorporated herein in their entirety.

### FIELD OF THE INVENTION

The present invention relates to immunology and more specifically to methods and compositions for modulating the stimulation of T cells and implications thereof for design of therapeutic strategies in vitro, ex vivo and in vivo.

### BACKGROUND OF THE INVENTION

The immune system acts as a defense against a variety of internal and external conditions which include, for example, infections, cancer, mutations, injuries and the like, and is mediated by two interconnected systems: the humoral and cellular immune systems. Briefly, the humoral system is mediated by the action of soluble molecules termed antibodies or immunoglobulins which, through their properties of specifically combining with a target (e.g., an antigen) recognized as being foreign to the body (or non-self), can inactivate same. The cellular immune system also involves the mobilization of cells, termed T cells. T cells are responsible for what is called cell-mediated immunity. This immunity involves the destruction of foreign cells, infected cells or the like by the action of cells of the immune system.

T cells can be subdivided into different subsets based on surface markers or based on function. For example, “helper”, “regulatory” and “killer” T cell subsets have been described. A T cell which recognizes and binds to a particular antigen displayed on the surface of another cell (often termed antigen presenting cell or APC) can become activated. An activated T cell can multiply, produce cytokines and, if it is a killer T cell, can kill the cell to which it is bound. Helper T cells generally produce cytokines and activate other cells of the immune system. Killer T cells recognize infected, foreign or altered cells, such as cancer cells and eliminate them. Regulatory T cells can modulate or suppress certain immune responses.

Different subsets of T cells can also be identified and are generally defined by the antigenic determinants found on their cell surfaces. Samples of such subsets include CD4 and CD8 T cells. “CD” refers to the cell differentiation cluster and the numbers accompanying same are in accordance with the terminology set forth by the international workshop on leukocyte differentiation. In general, CD4⁺ T cells recognize antigen as a peptide bound to an MHC class II protein on the surface of an antigen presenting cell and CD8⁺ T cells recognize antigen as a peptide complexed to MHC I proteins on the surface of an antigen presenting cell or target cells.

Cytotoxic or killer T cells are primarily found in the CD8⁺ T cell subset and “helper” T cells are primarily found in the CD4⁺ T cell subset.

Memory T cells are T cells that have been previously exposed to antigen and persists in the host ready to eliminate the foreign agent, infection or cancer if it appears again. A number of cell surface markers are associated with an activated or memory T cells. T cells in humans can also differ in their expression of the T cell surface protein CD28. CD28 is a surface receptor important in initial T cell activation. Humans possess both CD28⁺ and CD28⁻ T cells. Memory T cells are found in both the CD28⁺ and CD28⁻ T cell subset and it is not fully understood why some memory cells lose CD28 expression. However, it is clear that the number of CD28⁻ T cells are increased with age and in certain disease states such as HIV infection (up to 80% of T cells are CD28⁻), inflammatory arthritis, other auto-immune diseases and multiple myelomas.

The physiological mechanism of human T cell activation involves the recognition of an MHC-peptide complex by the antigen-specific T cell receptor together with other receptor ligand interactions, known as costimulatory interactions. However, a number of additional means can be used to stimulate T cells, such as antibodies to T cell surface receptors or mitogenic lectins. Of note, the induction of proliferation is only but one marker of T cell activation, since other markers include: increase in lymphokine or cytokine production, cytotoxic activity and a change in the basal or resting state of the cell.

The complex phenomenon of T cell activation involves a variety of receptor/ligand interactions between T cells and antigen presenting cells. One key player in T cell activation is the T cell receptor (TCR), a disulfide-linked heterodimer which contains two glycoprotein chains (α/β) uncovalently associated with a complex of low molecular weight invariant proteins which are commonly designated as CD3. While the TCR α and β chains (or γ and δ) determine the antigen specificities of the T cell, the CD3 structures of the TCR are thought to be responsible for transducing the activation signal upon binding of the α and β chains to its ligand. As discussed above, the TCR interacts with small peptidic antigens which are presented by the major histocomptability complex (MHC) proteins. The MHC proteins are a highly polymorphic set of molecules which are randomly dispersed throughout the species and further increase the complexity of the T cell activation phenomenon.

In summary, therefore, T cell activation usually requires a trimolecular interaction between a TCR, a peptidic antigen and MHC proteins which bind to this antigen. Although the recognition of antigen/MHC by the antigen-specific T cell is necessary for T cell activation, this signal alone is usually not sufficient to activate a T cell, rather, other receptor-ligand interactions, called costimulatory interactions are usually also required. The CD28 receptor on T cells, binding to B7 molecules on antigen presenting cells can provide such a costimulatory signal. However, since CD28 is not present on all human T cells, a critical issue remains how to activate the CD28⁻ T cells.

4-1BB is a costimulatory member of the tumor necrosis factor receptor (TNFR) family, expressed on activated CD4 and CD8 T cells (for review, see 1, 2). The 4-1BB ligand (termed 4-1BBL) is expressed on activated antigen-presenting cells (APC), including IFNγ-activated macrophages, Ig or CD40L-activated B cells as well as mature dendritic cells (3-5). There is an extensive body of literature indicating that murine 4-1BBL can augment T cell proliferation, cytokine production, cytolytic effector function and prevent activation induced cell death (1, 2). In the mouse system, using artificially generated CD28⁻ cells (5, 6), it was shown that when given in conjunction with a strong signal through the T cell receptor (TCR), 4-1BBL can induce IL-2 production by resting CD4 T cells independently of the CD28 costimulatory pathway. However, when signals through the TCR are limiting, CD28 is much more effective in costimulating IL-2 production by resting mouse T cells than is 4-1BB, likely due to the requirement for TCR-induced 4-1BB activation prior to the T cells becoming responsive (6). Although some studies have suggested a preferential role for 4-1BBL in CD8 T cell activation in mice (7, 8) a number of murine studies have shown that CD4 T cells also respond to 4-1BBL (6, 9-12). Recent experiments have shown that murine CD4 and CD8 T cells respond similarly to 4-1BBL (13). In contrast, agonistic anti-4-1BB antibodies show differences in stimulation of the two subsets (7, 13). For example, anti-4-1BB monoclonal antibodies have been shown to generally preferentially target CD8 cells having only a minor effect on CD4 T cell activation. In vivo, the 4-1BB/4-1BBL costimulatory pathway has been shown to augment suboptimal cytotoxic T-lymphocytes (CTL) responses to influenza virus and LCMV (14-16) and to augment anti-tumor immunity (17-21). Doubly deficient, CD28^{-/-} 4-1BBL^{-/-} knock-out mice show a delay in skin allograft rejection compared to mice lacking either one of these costimulatory molecules (14). In mouse models of graft versus host disease (GVHD), 4-1BB and 4-1BBL have been shown to play a role in both the CD4 and CD8 T cell component of the response (12).

Human 4-1BB (CD137) was cloned by three different groups (22-24), and has also been referred to as ILA (24). Human 4-1BB/ILA is 60% identical to murine 4-1BB, and contains notable differences in its cytoplasmic tail. In particular, human 4-1BB lacks the single tyrosine residue and also diverges from murine 4-1BB at the putative lck binding site found in the murine 4-1BB cytoplasmic tail (25). However, both human and murine 4-1BB have in common the motifs required for binding tumor necrosis factor receptor (TNFR) associated factor TRAF (23-28), an adaptor protein that is essential for mediating downstream signaling events leading to IL-2 production in response to 4-1BBL signaling (2, 29, 30). Human 4-1BB is expressed on activated CD4 and CD8 T cells. 4-1BB is expressed at higher levels on activated CD8 T cells from HIV⁺ individuals than on CD8 T cells from healthy donors. In addition to its expression on T cells, human 4-1BB expression has been reported on epithelial and hepatoma cells (24) as well as on blood vessels from individuals with malignant tumors (31). Interestingly, the human 4-1BB gene maps to human chromosome 1p36, a region previously associated with several malignancies (32). A soluble form of 4-1BB has also been reported in the serum of patients with Rheumatoid arthritis (33). Human monocytes also express 4-1BB and anti-4-1BB has been shown to augment TNFα and IL-8 production by monocytes (34). 4-1BB is also expressed on neutrophils and anti-4-1BB can ameliorate activation induced cell death of neutrophils (35).

4-1BBL-transfected CV1 cells or anti-4-1BB antibodies can augment PHA-stimulated or CD3-stimulated T cell proliferation, respectively (22, 36; U.S. Pat. No. 6,355,779 [Goodwin et al., Mar. 12, 2002]). For a human Th1 clone, the effects of anti-4-1BB were only observed in conjunction with CD28 signaling and 4-1BB was found to enhance proliferation and cytokine production by the anti-CD3+anti-CD28 stimulated cells (37). In contrast, murine 4-1BB-mediated costimulation is CD28-independent (5).

Human 4-1BBL is found on EBV-transformed human B cell lines as well as on the monocyte cell line THP-1. It shares 36% identity with murine 4-1BBL and like murine 4-1BBL is a type II glycoprotein with a single predicted transmembrane segment (22). Immobilized 4-1BBFc can induce monocytes to secrete cytokines, suggesting that human 4-1BBL may be involved in reverse signaling in APC (38, 39).

The binding of B7.1 or B7.2 present on antigen presenting cells to CD28 on T cells, together with signals through the TCR, provide signals for initial T cell activation (40, 41, 46). At birth, most human T cells express CD28. With age however, humans accumulate CD28⁻ T cells (43, 44). CD8 ⁺CD28⁻ T cells found in asymptomatic carriers of HIV and HCMV contain high frequencies of cells with specificity for viral epitopes (45-48). Furthermore, TCR gene analysis of CD8 ⁺CD28⁻ T cells suggests that CD28⁻ T cells are clonally related to CD28⁺ T cells (45). Analysis using MHC I/HIV peptide tetramers has shown that the memory CD8 T cell subset in HIV infected individuals is largely found in the CD28⁻ subset (46). Others have suggested that CD28⁻ T cells contain the active effector population, based on their expression of NK markers (49). CD8 ⁺CD28⁻ T cells lack immediate activation markers such as CD69, consistent with a memory phenotype (43).

The finding that CD28⁻ T cells have shortened telomeres and relatively poor responses to stimulation raises the question of whether they can be activated to further enhance the immune response or whether they represent senescent or terminally differentiated effector cells (50). Despite their limited proliferative potential, CD28⁻ T cells can be induced to divide in culture and in fact clones of CD8⁺ CD28⁻ and CD4⁺ CD28⁻ T cells have been generated (51). The propagation of such clones requires autologous feeder cells, implying a requirement for costimulatory ligands on APC (51). Costimulatory ligands capable of sustaining human CD28⁻ T cells have not been identified to date (51). CD28⁻ T cells have been shown to accumulate to a greater extent in certain disease states such as rheumatoid arthritis (RA; 52) haematopoietic cancers (53) and HIV infection (45-48).

4-1BB is an inducible member of the tumour necrosis factor receptor family found on activated CD4 and CD8 T cells as well as on some non-lymphoid tissues (22, 24, 54). Its ligand, 4-1BBL is expressed on activated APC (3, 5, 22). Work from a number of groups has shown that engaging 4-1BB with antibodies or ligand can activate murine CD4 and CD8 T cells from wild-type (WT) or gene targeted CD28^{-/-} mice to proliferate, secrete cytokines, develop CTL effector function and prolong their survival (1, 2, 55). In vivo, murine 4-1BBL has been shown to play a critical role in the memory CD8 T cell response to viruses as well as in graft rejection and MHC I- or MHC II-restricted graft versus host disease (12, 14-16, 56). Systemic administration of anti-4-1BB antibody in mice potentiates CD8 T cell survival and enhances tumor rejection (7, 8, 18).

4-1BB has also been shown to play a role in costimulation of human T cell responses (22, 57). To date, however, studies of 4-1BB mediated costimulation either did not eliminate the effects of CD28^{'1} B7 or found that T cell responses to 4-1BB ligation were dependent on CD28 (22, 57).

Thus, there are clear and significant differences between human and murine 4-1BB and 4-1BBL. One such significant difference is the fact that CD28 in mice is constitutively expressed in cell peripheral blood T cells. In contrast, CD28 expression in humans is significantly lowered with aging. In addition, it is affected in disease states. To date, studies with human 4-1BBL have largely depended on the use of antibodies and have examined proliferation of unfractionated T cells.

It has proven difficult in the past to activate human T cells to kill target cells. An example of a method for activating T cells is provided in U.S. Pat. No. 6,406,696 (Bluestone, Jun. 18, 2002), which describes a method for activating T cells via the in vivo administration of a soluble anti-CD3 monoclonal antibody.

Another method to stimulate T cells is taught in U.S. Pat. No. 6,355,779 (Goodwin et al., Mar. 12, 2002) which teaches that 4-1BBL transfected into peripheral blood T cells in the presence of PHA as a costimulus enhanced T cell proliferation under suboptimal PHA concentrations, and that hv4-1BBL had no effect on T cell proliferation in the absence of this PHA costimulus. PHA is known to be a non-specific ligand, a lectin that binds to oligosaccarides on the T cell surface, and as such the enhanced proliferation is thus likely to be non-specific, since PHA can bind to numerous receptors. Of note, a related lectin, concanavalin A, has been shown to exert its mitogenic effect in part through an interaction with the CD28 receptor (68) (CD28⁺-dependent).

Efficient activation of the cellular arm of the immune system requires a specific TCR signal delivered through peptide/MHC complexes together with costimulatory signals delivered by constitutive or inducible costimulatory molecules. While the best known costimulatory molecule is CD28 (41), recently other inducible costimulatory molecules have been characterized (74, 2, 83). The emerging picture is that CD28 is important for the initial activation of an immune response, and other costimulatory ligand-receptor pairs act later, to help sustain and diversify the response (74, 2, 83). Recent evidence suggests that 4-1BB/4-1BBL interaction plays an important role in the memory CD8 T cell responses to viruses (14, 16, 56).

4-1BB is an inducible costimulatory member of the TNFR family expressed on activated CD4 and CD8 T cells (reviewed in (1)). Its ligand, 4-1BBL (2), is expressed on activated APC (3-5 and 84). 4-1BB can enhance both the proliferation and survival of murine CD4 and CD8 T cells (3, 5, 8-11, 13, 85 and 86),. Although 4-1BBL can stimulate both CD4 and CD8 T cells (9, 11, 13, and 87), anti-4-1BB antibodies preferentially stimulate CD8 T cells (7). Mice lacking 4-1BBL have a defect in the CD8 recall responses to viruses, with no detectable effect on the anti-viral CD4 T cell or anti-viral antibody responses (14-16). Whereas CD28 is critical for primary expansion of viral specific CTL, the absence of 4-1BBL results in normal primary expansion and contraction of CD8 T cells in response to influenza virus. However, in the absence of 4-1BBL there are decreased numbers of CD8 T cells late in the primary response as well as a defect in secondary responses upon challenge in vivo (25).

In several mouse models, anti-4-1BB or 4-1BBL have shown promise in anti-tumor vaccination strategies (18-20, 79, 81, 88, 89, 90) Anti-4-1BB has been shown to promote the regression of tumors, an effect that was dependent on the presence of CD4, CD8 and NK cells (19, 91). 4-1BB-mediated costimulation is independent of CD28 and can allow expansion of CD28^{-/-} T cells both in vitro and in vivo (5, 6, 92).

Human 4-1BBL or anti-4-1BB plus anti-CD3 can stimulate both CD4 and CD8 CD28⁺ or CD28⁻ T cells to divide and upregulate their effector function (22, 80, 37, 58, 93, 94) The inclusion of 4-1BBL together with anti-CD3/anti-CD28 has also been shown to increase yields of human CD8 T cells in long term cultures (57). The accumulation of CD28⁻ T cells in chronic viral disease (61, 95) and in aging (44) suggests that under such conditions, 4-1BBL might be better suited for boosting immunity than B7.1 (96).

To date, studies of human T cell responses to 4-1BBL have involved polyclonal T cell activation models or have not tested the effects of 4-1BBL in isolation (57). In one study, anti-4-1BB was used to augment priming of peptide-specific T cell responses from cord blood but with only modest effect (80). In view of the greater effects of 4-1BBL on secondary versus primary CD8 T cell responses, in mice (56).

There thus remains a need to provide a means and compositions thereof for activating human T cells, thereby allowing T cell expansion as well as cytokine production and the development of CTL effector function. There also remains a need to provide a means and compositions thereof to deactivate human T cells. In particular, there remains a need to modulate the activation of human CD28⁻ T cells. As well, there remains a need to provide a strategy to augment human MHC-restricted responses.

### SUMMARY OF THE INVENTION

The present invention relates to immunology and more specifically to methods and compositions for modulating the stimulation of T cells in vitro and implications thereof for design of therapeutic strategies in vitro, ex vivo and in vivo. In embodiments, the invention relates to primate cells, proteins and/or nucleic acids. In embodiments, the invention relates to human cells, proteins and/or nucleic acids.

In a first aspect, the invention provides a method of preparing an antigen presenting cell specific to an antigen, said method comprising: (a) providing a human monocyte or monocyte-related cell; (b) increasing the level of a 4-1BBL in said monocyte or monocyte-related cell; (c) increasing the level of a B7 molecule in said monocyte or monocyte-related cell; (d) contacting said monocyte or monocyte-related cell with said antigen or a part thereof; and (e) culturing said monocyte or monocyte-related cell for less than 1 week thereby to allow its conversion to an antigen presenting cell.

In an embodiment, the B7 molecule is selected from the group consisting of B7.1 and B7.2.

In an embodiment, the level of a 4-1BBL is increased by introducing into said monocyte or monocyte-related cell a nucleic acid encoding said 4-1BBL. In an embodiment, the level of a B7 molecule is increased by introducing into said monocyte or monocyte-related cell a nucleic acid encoding said B7 molecule. In an embodiment, the nucleic acid is introduced into said monocyte by introducing into said monocyte a vector (e.g. a recombinant vector) comprising said nucleic acid. In an embodiment, the vector is a viral vector, in a further embodiment, an adenovirus.

In an embodiment, the nucleic acid encoding said 4-1BBL is introduced into said monocyte or monocyte-related cell by introducing into said monocyte or monocyte-related cell a vector comprising said nucleic acid encoding said 4-1BBL, and wherein said nucleic acid encoding said B7 molecule is introduced into said monocyte or monocyte-related cell by introducing into said monocyte or monocyte-related cell a vector comprising said nucleic acid encoding said B7 molecule.

In embodiments, the nucleic acids noted herein may be located within or introduce via the same vector or separate vectors.

In an embodiment, the vector is a viral vector, in a further embodiment, an adenovirus.

In an embodiment, the method further comprises contacting said T cell with a TNF ligand. In an embodiment, the TNF ligand is selected from the group consisting of OX40L, LIGHT, CD70, CD30 and GITR-L.

In embodiments, the culturing step (e) has a duration of less than about 72 hours, in a further embodiment less than about 24 hours, in a further embodiment less than about 16 hours, in a further embodiment from about 12 to about 16 hours.

In an embodiment, the antigen or part thereof is selected from a virus, a protein and a polypeptide. In an embodiment, the protein or polypeptide is contacted with said monocyte or monocyte-related cell by introducing into said monocyte or monocyte-related cell a nucleic acid capable of encoding said protein or said polypeptide.

The invention further provides an antigen presenting cell produced by the above-mentioned method, wherein said antigen presenting cell comprises: (a) a recombinant vector comprising said nucleic acid encoding said 41BBL; and (b) a recombinant vector comprising said nucleic acid encoding said B7 molecule. In embodiments, both said nucleic acid encoding said 41BBL and said nucleic acid encoding said B7 molecule may be located within or comprised in the same or separate vectors.

The invention further provides a composition comprising the above-mentioned antigen presenting cell and a pharmaceutically acceptable carrier.

The invention further provides a vaccine comprising the above-mentioned antigen presenting cell.

The invention further provides a method of activating a human T cell, said method comprising contacting said T cell with the above-mentioned antigen presenting cell. In an embodiment, the contacting step is carried out ex vivo. In an embodiment, the contacting step is carried out in vivo and said antigen presenting cell is administered to a subject comprising said human T cell.

In an embodiment, the human T cell is obtained from a subject suffering from a condition associated with immuno impairment.

The invention further provides method of treating a subject suffering from a condition associated with immuno impairment, said method comprising administering to said subject the above-mentioned antigen presenting cell.

The invention further provides a method of vaccinating a subject, said method comprising administering to said subject the above-mentioned antigen presenting cell.

The invention further provides a method of treating a subject suffering from a condition associated with immuno impairment, said method comprising administering to said subject an activated T cell prepared by the above-mentioned method.

In an embodiment, the above-mentioned condition is selected from the group consisting of viral disease, pathogen infection and cancer. In an embodiment, the viral disease is selected from the group consisting of AIDS, hepatitis C, and CMV-related disease. In an embodiment, the pathogen is selected from the group consisting or a bacteria, a fungus and a parasite.

In an embodiment, the above-mentioned antigen is associated with a complex comprising an MHC I or MHC II molecule and said complex is presented on the surface of an antigen presenting cell.

In an embodiment, the 4-1BBL is substantially identical to SEQ ID NO: 2. In an embodiment, the 4-1BBL is encoded by a nucleic acid selected from the group consisting of: (a) a nucleic acid capable of encoding SEQ ID NO: 2; (b) SEQ ID NO: 1; and (c) a nucleic acid substantially identical to (a) or (b).

In an embodiment, the B7.1 is substantially identical to SEQ ID NO: 4. In an embodiment, the B7.1 is encoded by a nucleic acid selected from the group consisting of: (a) a nucleic acid capable of encoding SEQ ID NO: 4; (b) SEQ ID NO: 3; and (c) a nucleic acid substantially identical to (a) or (b).

In an embodiment, the B7.2 is substantially identical to SEQ ID NO: 6. In an embodiment, the B7.2 is encoded by a nucleic acid selected from the group consisting of: (a) a nucleic acid capable of encoding SEQ ID NO: 6; (b) SEQ ID NO: 5; and (c) a nucleic acid substantially identical to (a) or (b).

The invention further provides uses and commercial packages relating to the products and methods mentioned herein. Accordingly, the invention further provides a use of 4-1BBL, a B7 molecule, and an antigen or part thereof, optionally in the form of nucleic acid(s) which encode these component(s), for the preparation of an antigen presenting cell, according to the methods described herein. The invention further provides a commercial package comprising a 4-1BBL, a B7 molecule, an antigen or part thereof, optionally in the form of nucleic acid(s) which encode these component(s), and instructions for the preparation of an antigen presenting cell, in accordance with the methods described herein. The invention further provides a use of the above-mentioned antigen-presenting cell for (or for preparation of a medicament for) activation of a T cell, for treatment of a disease associated with immuno impairment, or for vaccination, in accordance with the methods described herein. The invention further provides a commercial package comprising the above-mentioned antigen-presenting cell and instructions for (or for preparation of a medicament for) activation of a T cell, for treatment of a disease associated with immuno impairment, or for vaccination, in accordance with the methods described herein. The invention further provides a use of an activated T cell prepared using the methods described herein for (or for preparation of a medicament for) treatment of a disease associated with immuno impairment. The invention further provides a commercial package comprising an activated T cell prepared using the methods described herein for (or for preparation of a medicament for) treatment of a disease associated with immuno impairment.

The present invention further relates to methods for modulating human CD28⁻ T cells. In an embodiment, the invention relates to human CD28⁻ T cells activation resulting in cell division, cytokine production, enhancement of cytolytic effector function as well as to the inhibition of the apoptotic pathway in these cells. The present invention also relates to an inhibition of human T cells and particularly CD28⁻ T cell activation. Further, the present invention finds utility in a variety of diseases or conditions in humans and particularly those in which CD28⁻ T cells are increased in numbers, such as in chronic viral infection, cancer and autoimmune disease.

The invention further relates to a method of activating human T cells against specific antigens in vitro, so that they can be reinfused into patients to fight a predetermined disease or condition in a patient, comprising:

a) upregulating 4-1BB expression in said human T cells ; and

b) stimulating said T cells with an antigen presenting cell which provides in combination with MHC/Ag and/or thereby activating said T cells prior to said reinfusion in said patient. In an embodiment, the disease or condition is cancer or AIDS.

The invention further relates to a method to induce human CD4 and/or CD8 T cell expansion, and/or to enhance cytokine production and/or to augment the development of cytotoxic effector function in said T cells, comprising expression in said human T cells and providing and a signal through the T cell receptor of said human T cells on the same stimulatory antigen presenting cell (APC).

The invention further relates to a use of an antigen-presenting cell which expresses 4-1BBL for expanding the CTL function of a human T cell with concomitant development of CTL effector function thereof.

The invention further relates to a method of expanding T cells in culture comprising an incubation of human CD4 and/or CD8 T cells with 4-1BBL and a signal which stimulates the T cell receptor the thereby enabling a co-stimulation which enhances the expansion of said T cells in culture. In an embodiment, the CD4 and CD8 T cells are present in the same culture.

The invention further relates to a method to augment human MHC-restricted responses using a composition which upregulates 4-1BB rapidly, said composition comprising at least one of a FcR bearing, 4-1BBL-transfected APC that can present both the APC-bound molecule which upregulates 4-1BB and an MHC-peptide combination of interest, and a 4-1BBL molecule to stimulate said T cells through 4-1BB. In an embodiment, the molecule which upregulates 4-1BB rapidly is OKT3.

In an embodiment, anti-CD3 is used in conjunction with 4-1BBL for expanding functional CD4 T cells and CD8 T cells with cytotoxic activity.

In an embodiment, the expansion is performed when CD4 and CD8 T cells are present in the same culture.

The invention further relates to a method of stimulating and/or production in human T cells comprising an upregulation of 4-1BB expression in said human T cells, in combination with stimulation with and a signal through the wherein said T cell activation is accompanied by an increased production of said IL-2 and/or INF-γ. The method of claim 11, wherein said upregulation is performed in the presence of OKT3.

In embodiments, the stimulation is carried-out by a delivery of human 4-1BBL into antigen presenting cells. In an embodiment, the delivery of human carried-out by a transfection of said APC of a nucleic acid encoding a functional 4-1BBL. In an embodiment, 4-1BBL protein is delivered. In embodiments, the nucleic acid is a delivered into APC cells by electroporation or a viral vector.

The invention further relates to an in vivo vaccination strategy comprising a co-delivery of and a T cell activating signal to antigen presenting cells, whereby the 4-1BBL and said signal are delivered to an antigen presenting cell (APC). In an embodiment, the T cell activating signal is selected from a protein, a peptide, an epitope, an antigen or pool thereof, or a nucleic acid sequence encoding same. In an embodiment, the antigen or pool thereof is a specific antigen or pool of specific antigens. In an embodiment, the 4-1BBL and the T cell activating signal are co-delivered to the same APC. In an embodiment, the 4-1BBL and the T cell activating signal are on the same viral vector.

The invention further relates to a composition for activating human T cells for inducing human CD4 and/or CD8 T cell expansion, and/or to enhance Th1 cytokine production, and/or to augment the development of effector function in said T cells, and/or for expanding the CTL function of said T cells, and/or to augment human MHC-restricted responses, and/or to stimulate and/or IFN-γ production in said T cells, comprising subjecting said T cells to a biologically active amount of and a sufficient amount of a molecule that binds the T cell receptor of said human T cells and upregulates 4-1BB in said T cells. In an embodiment, the composition is a vaccine.

In embodiments, the methods of the invention may be effected in vitro, in vivo and ex vivo.

### BRIEF DESCRIPTION OF THE DRAWINGS

Having thus generally described the invention, reference will now be made to the accompanying drawings, showing by way of illustration a preferred embodiment thereof, and in which:

FIG. 1 shows the induction of human 4-1BB on peripheral blood lymphocytes from healthy donors following stimulation with immobilized anti-CD3. Total T cells were analyzed by three colour flow cytometry after staining with anti-CD3, anti-CD4 or anti-CD8 and anti-4-1BB.

FIG. 2 shows the expression of 4-1BBL on mock transfected versus 4-1BBL-transfected P815 cells. Cloned P815 cells after human 4-1BBL or mock (pcDNA3) transfection were stained with PE-conjugated anti-human 4-1BBL mAb and analyzed for human 4-1BBL expression on a Becton-Dickinson Facscalibur™.

FIG. 3 shows the expansion of T cells following 4-1BBL mediated costimulation. A) 1.3×10⁶ total T cells, CD4 T cells or CD8 T cells were incubated with 0.7×10⁶ irradiated 80Gy 4-1BBL or mock-transfected P815 stimulator cells with or without OKT3 in a 2 ml culture as indicated in the figure. After 5 days of culture, cells were recovered and viable cell numbers assessed by trypan blue exclusion. In a parallel experiment, B), samples were analyzed by flow cytometry to determine the proportion of CD4 and CD8 T cells using FITC-anti-CD4 and PE-conjugated CD8 following various treatments, as well as for scatter parameters as an indication of whether cells had undergone blastogenesis in the cultures. Stimulators: a. P815, b. P815+OKT3, c. P815-H4-1BBL, d. P815-H4-1BBL+OKT3. Control staining with isotype control antibodies was used to define the gates. This experiment is representative of four similar experiments.

FIG. 4 shows the role of 4-1BBL in induction of IL-2 production by CD4, CD8 or total T cells. Total T lymphocytes or isolated CD4 or CD8 T cells were stimulated as in FIG. 3A, using mock transfected or human 4-1BBL-transfected P815 cells, with or without OKT3, as indicated in the figure. After 48 hr, supernatants were removed and analyzed for IL-2 content using a CTLL-2 bioassay as described in the examples below. This experiment is representative of four similar experiments.

FIG. 5 shows the IFN-γ production in response to 4-1BBL mediated stimulation. 10⁵Purified T cells were cultured in 200 ml CCM in 96-well plates with 5×10⁴ irradiated control or H4-1BBL-transfected P815 cells at a 2:1 ratio for 2 to 4 days. Human IFN-γ in the supernatant was measured by ELISA. This experiment is representative of two similar experiments.

FIG. 6 shows the role of 4-1BBL in augmenting the development of Cytotoxic effector function. Purified total T responders were co-cultured with irradiated stimulators at a 2:1 ratio for 5 days. OKT3-loaded stimulator cells were prepared as described below. The results presented are based on triplicate samples and this experiment is representative of three similar experiments.

FIG. 7 shows the expression of CD28 and 4-1BB on T cells. a. Unfractionated T cells were stimulated with plate-bound anti-CD3 for the duration indicated, gated on CD4⁺ or CD8⁺ T cells, and plotted as CD28 vs. 4-1BB dot plots (representative of 7 donors). The numbers in each quadrant indicate the percentage of gated cells in that quadrant b. The frequency of CD28⁻ T cells as a function of time was measured by flow cytometry for four different donors.

FIG. 8 shows the role of 4-1BBL in expansion of CD28⁺ and CD28⁻ T cells. a. Model system utilized. 4-1BBL transfected P815 cells or control transfected cells, coated with anti-CD3 antibody (OKT3) were used to stimulate unfractionated or sorted CD28⁺ and CD28⁻ T cells. b. Unfractionated T cells from healthy donors were stimulated for up to 96h with P815 cells with or without 4-1BBL in the presence or absence of anti-CD3, as indicated on the left of the figure. The cell populations were gated on the four different subsets as indicated above each set of panels. Data are representative of results from 3 donors. c. Purity of sorted CD28⁺ and CD28⁻ T cells after depletion of other subsets by FACS. Cells were stained for CD28 and CD3. The donor shown represents average purity obtained. d. Cell enumeration after stimulation with P815 cells±anti-CD3±4-1BBL as described in FIG. 2a. CD28⁺ and CD28⁻ T cells were separated by flow cytometry and stimulated for 5 days with P815 only (open bars), P815+ anti-CD3 (grey), P815-4-1BBL (hatched) or P815-4-1BBL plus OKT3 (black). The starting number was 1.5 million in all cultures. Live cells were counted, based on trypan blue exclusion (representative of 3 donors).

FIG. 9 shows the measurement of IL-2 production and IL-2 receptor expression by CD28⁻ T cells. a. IL-2 levels in the supernatant were measured using the IL-2 dependent cell line CTLL-2. Results are reported as tritiated thymidine incorporation in response to serial dilutions of stimulated culture supernatants. b. Expression of IL-2Rα on activated CD28⁺ and CD28⁻ T cells as measured by flow cytometry. Numbers in each quadrant represent the percent of CD28⁺ or CD28⁻ T cells positive for CD25 (data are representative of 4 donors).

FIG. 10 shows that 4-1BB costimulation enhances the level of BCL-X_{L} in both CD28⁺ and CD28⁻ T cells. The Bcl-X_{L} level in cells, stimulated as indicated above each panel, was measured by intracellular staining and flow cytometry. The numbers in each quadrant represent the percent of each population CD28⁺ or CD28⁻ that were positive for Bcl-X_{L} above basal level (data are representative of 3 donors).

FIG. 11 shows the CD28⁻ T cells produce IFN-γ in response to 4-1BBL-mediated costimulation. a. Unfractionated (Total) or sorted CD28⁺ and CD28⁻ T cells (as indicated on the left) were analyzed for IFN-γ production by intracellular staining following stimulation. Stimulation conditions are indicated above the panels (data are representative of 5 donors). For the donor shown, the CD28⁻ T cells were more than 95% CD8⁺. b. Production of IFN-γ was measured by ELISA for the populations indicated above each figure. Cells were stimulated with P815 alone (m), P815+anti-CD3 (1), P815-4-1BBL (o) or P815-4-1BBL plus anti-CD3 (n). Data are representative of results obtained from 4 donors. C. Expression of TNF-α by CD28⁺ and CD28⁻ T cells was followed by intracellular staining and flow cytometry after stimulation as indicated above each panel. Results are shown for TNFα versus CD28 staining. The numbers in the upper quadrants represent the percentage of each subset (CD28⁺ or CD28⁻) positive for TNFα expression over isotype control (data are representative of results obtained with 3 donors).

FIG. 12 shows that 4-1BBL-mediated costimulation increases the levels of perforin and cytotoxic activity of CD28⁺ and CD28⁻ T cells. a. Levels of perforin were analyzed by intracellular staining of unfractionated, freshly isolated T cells. Isotype control (thin line), CD28^{'} (thick black line), and CD28⁺ (thick grey line) are plotted after electronic gating on the CD4 or CD8 T cell subset. The donor shown has a significant population of CD4⁺ CD28⁻ T cells, not a common occurrence. The CD8⁺ CD28⁻ population predominates in the majority of donors (representative of 5 donors). b. Unfractionated (⋄) or sorted CD28^{'1} (n) or CD28⁺ (m) T cells were incubated with P815 targets, with or without anti-CD3, as indicated above each panel. c. Sorted CD28⁺ or CD28⁻ T cell subsets were stimulated with P815 alone (m), P815 coated with anti-CD3 (1), P815-4-1BBL (o) or P815-4-1BBL coated with anti-CD3 (n). Five days later, the stimulated cells were tested for lysis of anti-CD3-coated P815 cells in a 4 hr ⁵¹Cr-release assay. d. Following the 5-day stimulation, as in FIG. 6c, the level of perforin in sorted CD28⁺ or CD28⁻ was followed by intracellular staining and flow cytometry. Cells are gated on each T cell subset and isotype control (dashed), P815-4-1BBL+anti-CD3 stimulated cells (black) and P815+anti-CD3 stimulated cells (grey) are plotted (representative of 3 independent donors).

FIG. 13 shows 4-1BBL expression in human blood cells using a replication defective viral vector. Transduction of peripheral blood adherent cells with adenovirus encoding the human 4-1BBL gene was carried out as follows. Blood mononuclear cells from a healthy HLA-A2 positive donor were allowed to adhere to plastic to remove lymphocytes, at a concentration of 5×10⁶/ml for 2 hours and washed 2× with media. The adherent cells (mainly monocytes) were mixed with control adenovirus or adenovirus encoding human 4-1BBL. To obtain greater transduction efficiency, the adherent cells were centrifuged for 1 h 30min at 1350×g at 35° C. Cell viability was not affected, as judged by negative 7-AAD staining up to 3 days after the centrifugation procedure. Expression was 25% at 24h, and 34% at 48h, as visualized by FACS staining for 4-1BBL (FIG. 13B). Control adenovirus induced negligible amounts of 4-1BBL (FIG. 13A).

FIG. 14 shows the effect of a costimulation of peptide-specific HLA-A2-restricted responses of purified total T cells by 4-1BBL of FIG. 13. Dashed lines show responses to control adenovirus while solid lines correspond to 4-1BBL containing adenovirus+peptide. T cells were purified from the non-adherent lymphocytes described above and shown in FIG. 13 by using Cellect™ Enhanced Total T cell immunocolumns (Cedarlane Laboratories). The purified T cells were then incubated with adenovirally transduced autologous adherent cells+10 μm of influenza or EBV peptides or an equivalent amount of DMSO for the time indicated and 5 U/ml IL-2. Cells were restimulated on day 3 and day 6 with media containing 5 μm peptide/DMSO and 5 U/ml IL-2. IFN-γ ELISA was performed on supernatants obtained 3 and 6 days after the stimulation.

FIG. 15: Use of Adenovirus to deliver 4-1BBL (and/or B7.1) to syngeneic APC for use in peptide-specific T cell activation.

This figure shows the methodology for converting peripheral blood monocytes from patients or healthy donors into antigen presenting cells using recombinanant adenovirus and peptide antigens as described in Example 17. This method has been tested with HLA-A2+ healthy donors with Influenza Matrix Protein (GILGFVFTL [SEQ ID NO: 7]) and EBV BMLF1 protein (GLCTLVAML (SEQ ID NO: 8]) epitopes and with HIV donors of A2 and B8 HLA type using two different peptide epitopes.

FIG. 16 shows the characterization of adherent cells and gene transfer efficiency with recombinant adenoviruses as described in Example 18. Adherent cells are monocytes and express 4-1BBL and B7 24 h post-infection. The modified or control adenovirus also increases levels of HLA class I and II molcules which also increaseantigen presenting cell function.

FIG. 17 shows the effect of 4-1BBL on expansion of T cells from healthy donors. 4-1BBL acts as a potent adjuvant for influenza- and EBV-specific memory CD8 T cell responses as described in Example 19 . In healthy donors, 4-1BBL alone allows expansion of virus peptide specific T cells. The data shows expansion of Influenza-M1 (GILGFVFTL [SEQ ID NO: 7]) specific T cells by 4-1BBL costimulation in A*0201⁺ donor: Similar results were found for EBV specific responses also.

FIG. 18 shows studies of the ability of 4-1BBL-expanded T cells to kill influenza peptide-treated cells as described in Example 20. For healthy donors, adherent cells modified with peptide+4-1BBL adenvoirus alone enhances cytolytic activity of virus-specific T cells. Chromium release assay: Targets are T2 cells (HLA-A*0201+) pulsed with Influenza M1 peptide or DMSO.

FIG. 19 shows an induction of cytokine production by 4-1BBL-expressing APC as described in Example 21. On restimulation, 4-1BBL costimulated T cells produce IFNγ, TNFα and some IL-2.

FIG. 20 shows a comparison of 4-1BBL and B7.1 or the combination for stimulation of influenza specific CD8 T cells as described in Example 22. Either B7 or 4-1BBL-modified APC+peptide can expand influenza specific CD8 T cells from healthy donors and there is no added benefit of combining the two, and in fact the combination leads to slight inhibition.

FIG. 21 shows a comparison of 4-1BBL and B7.1 or the combination for stimulation of EBV-specific responses, in a study similar to FIG. 20, as described in Example 22. Either B7 or 4-1BBL -modified APC+peptide can expand EBV-1 specific CD8 T cells from healthy donors and there is no added benefit of combining the two, and in fact the combination leads to slight inhibition.

FIG. 22 shows that 4-1BBL is equal or better than B7.1 in increasing expression of survival and effector molecules in virus specific T cells from healthy donors, as described in Example 23. For healthy donor T cells, 4-1BBL is equal or slightly better than B7.1 in inducing effector molecule upregulation. Data shown for 3/5 donors; the other two donors showed similar levels of upregulation.

FIG. 23 shows that for HIV infected donors, the combination of B7+4-1BBL gives improved expansion of T cells and greatly improved induction of HIV peptide specific cytotoxic T cell activity over either stimulus alone, as described in Example 24. For HIV infected donors, the combination of B7+4-1BBL gives improved expansion of T cells and greatly improved induction of HIV peptide specific cytotoxic T cell activity over either stimulus alone. HIV Patient Responses: 9-day A2.1-SLYNTVATL (SEQ ID NO: 9) stimulation. Lower panel: Chromium release Assay: A2.1-SLYNTVATL (SEQ ID NO: 9) or irrelevant peptide targets.

FIG. 24 shows results of a study similar to that of FIG. 23, in this case using an HLA-B8 donor (see Example 24). HIV Patient Responses: 8-day B8-FLKEKGGL (SEQ ID NO: 10) stimulation. Lower panel: Perforin is upregulated most efficiently with the combination of B7 and 4-1BBL costimulation. Grey line: cells stimulated with APC expressing both B7 and 4-1BBL. Dashed line: cell line stimulated with control APC only. Thick and thin black lines represent B7 or 4-1BBL alone.

FIG. 25 shows the cytotoxic T cell activity against HIV peptide coated targets of the T cells generated in FIG. 24 (see Example 24). HIV Patient Responses: 8-day B8-FLKEKGGL (SEQ ID NO: 10) stimulation. Cells from the 8 day culture in FIG. 24 were used in a 4 hr ⁵¹Cr release assay to test lysis of HIV peptide coated cells.

FIG. 26 shows studies of an HIV patient for responses to influenza virus (see Example 24). HIV patient response to influenza virus peptides also shows the same dependence on both 4-1BBL and B7 expression on the APC.

FIG. 27: Fold-expansion in five healthy donors to influenza and EBV epitopes, showing lack of enhancement with the combination of 4-1BBL and B7.1 costimulation, as described in Example 25.

FIG. 28: Fold-expansion in six HIV patients to HIV or influenza epitopes, showing additive/synergistic effects with the combination of 4-1BBL and B7.1 costimulation (chronic patients), as described in Example 26. Length of infection and epitope are indicated.

FIG. 29 shows studies of other members of the TNF family, delivered by adenoviruses to human monocytes, with respect to enhancement of the ability of monocytes to activate influenza-specific T cells, as described in Example 27. Other members of the TNF ligand family are also able to promote expansion of influenza-specific CD8 T cells specific T cells from healthy donors. Therefore, it should be possible to substitute OX40L or LIGHT for 4-1BBL or use them in combinations to get further activation of T cell responses. In this example, adherent cells from healthy donors were incubated with B7, OX40L or LIGHT recombinant adenoviruses together with influenza peptide and seven days later, expansion of the T cells was monitored by MHC tetramer staining.

FIG. 30 shows human 4-1BBL DNA (SEQ ID NO: 1) and polypeptide (SEQ ID NO: 2) sequences (GenBank Accession U03398).

FIG. 31 shows human B7.1 DNA (SEQ ID NO: 3) and polypeptide (SEQ ID NO: 4) sequences (GenBank Accession NM_{—}005191).

FIG. 32 shows human B7.2 DNA (SEQ ID NO: 5) and polypeptide (SEQ ID NO: 6) sequences (GenBank Accession NM_{—}175862).

FIG. 33: 4-1BBL constimulated tetramer cells express higher levels of perforin, Granyzme A and Bcl-XL than control adenovirus stimulated cells. Intracellular levels of Perforin, Granzyme-A, Bcl-x_{L}, and Bcl-2 were measured by intracellular flow cytometry. 4-1BBL-Adv costimulated cultures (thick line) and control-Adv (thin line) are shown. All plots are gated on CD8⁺ Influenza-Tetramer⁺ T cells. Mean Fluorescence intensities of CD8 tetramer⁺ population for 4-1BBL (top number) and control adenovirus cultures (bottom number) are indicated beside the histograms

FIG. 34: 4-1BBL costimulation results in the generation of mature CD27-effectors that maintain CD28 expression. Prestimulation: Expression of CD27 and CD28 on Influenza Tetramer⁺ CD8 T cells in freshly isolated, unstimulated T cells from a healthy donor. Right panels: Expression of CD27 and CD28 on Tetramer+ T cells after a seven day stimulation with 4-1BBL (top) or control Adv (bottom) plus peptide. Data are gated on CD8⁺ Tetramer+ T cells. The percent of cells in each quadrant is indicated.

FIG. 35: Time course of expansion: 4-1BBL expands memory T cells from healthy donors with more rapid kinetics than B7.1. Kinetics of expansion of tetramer⁺ T cells in cultures with indicated recombinant adenoviruses as a function of stimulation time in days.

FIG. 36: 4-1BBL generates more mature CD27-effectors than B7.1 costimulation. T cells were cultured with adherent monocytes pre-incubated with the indicated recombinant adenoviruses and pulsed with Influenza matrix protein 1 peptide at 0.5 mM. Eight days later, cells were analyzed for CD27 and CD28 expression (data shown are gated on CD8⁺ Tetramer⁺ cells). Pre-stimulation profile is shown in the top two plots.

FIG. 37: Both effector and central memory cells are generated in response to either 4-1BBL or B7 costimulation. T cells were cultured with adherent monocytes pre-incubated with the indicated recombinant adenoviruses and pulsed with Influenza or EBV peptides as indicated. Expression of CCR7 and CD45RA for three donors, before and after stimulation. Pre-stimulation expression is shown on the left. Data shown are gated on CD8+ Tetramer+ cells, with the percentage of cells indicated in each quadrant.

Other objects, advantages and features of the present invention will become more apparent upon reading of the following non-restrictive description of preferred embodiments with reference to the accompanying drawings which is exemplary and should not be interpreted as limiting the scope of the present invention.

### DETAILED DESCRIPTION OF THE INVENTION

To study the role of 4-1BBL in human T cell responses, a model system was set up to stimulate human T cells with 4-1BBL and/or B7 in conjunction with a TCR signal. In some embodiments, CD28 negative T cells were activated, as such, in an embodiment, and in cases where 4-1BBL is used, the T cell may be a CD28⁻ T cell.

Further, among the studies described herein, the ability of 4-1BBL to stimulate anti-viral cytotoxic memory T cells from humans was tested. The results show that 4-1BBL is highly effective in expanding and activating T cell memory responses to influenza virus and EBV and is more effective than B7.1 in driving the CD8 T cells toward a fully differentiated CD27 effector phenotype. Although 4-1BBL or B7.1 expressed on donor monocytes were both capable of expanding virus-specific T cells, the combination did not result in enhanced responses. In contrast, when the same analysis was done on cells from HIV patients, then the combination of both B7.1 and 4-1BBL resulted in greater expansion of virus-specific T cells and greater cytotoxic activity against target cells coated with viral peptides. This requirement for both costimulatory molecules was observed with the HIV patient T cells whether one looked at HIV-specific or influenza-specific responses. Thus HIV infected individuals have a greater requirement for activation of their memory T cell responses than T cells from healthy donors.

Broadly, the present invention relates to methods and compositions to activate human T cells comprising a costimulation of the T cell using 4-1BBL fragment or variant thereof and a costimulus of TCR which is general (e. g. anti-CD3) or specific (e.g. specific antigen). The present invention therefore relates to an upregulation of 4-1BB in a T cell, together with a costimulation thereof with 4-1BBL, thereby activating same. In one embodiment, the activated T cell is a resting T cell or a CD28⁻ T cell.

In one embodiment, the present invention thus relates to methods and compositions to induce human T cell expansion, e.g. human CD4 and/or CD8 cell expansion, to enhance Th1 cytokine production and the development of cytotoxic effector function.

In another embodiment, the present invention relates to an induction of a response by the human T cells to a costimulator, for example an induction of a response to 4-1BBL such that the human T cells receive a signal through the TCR which upregulates 4-1BB, thereby enhancing the ability of 4-1BBL's biological action.

In addition, in another embodiment, the invention relates to an effect of a costimulator, e.g. 4-1BBL, on human T cells in culture containing both CD4 and CD8 T cells.

In yet another embodiment of the present invention, there is provided the identification that human 4-1BBL can promote CD28-independent human T cell activation.

In another embodiment, the invention relates to a use of a costimulator, e.g. 4-1BBL, for expanding the CTL with concomitant development of CTL effector function in human T cells.

In addition, in another embodiment, the present invention relates to a method of expanding human T cells in culture and composition for doing same, comprising an incubation of CD4 and CD8 human T cells with e.g. a 4-1BB ligand (e.g. 4-1BBL), and antibodies to TCR (general) or a specific antigen, thereby enabling a co-stimulation which enhances the expansion of the T cells in culture. In one particular embodiment, CD28⁺ cells are thereby activated. Moreover, in another embodiment, the present invention relates to a strategy to augment human MHC-restricted responses using a combination of molecules which upregulate 4-1BB rapidly (e.g. OKT3), which comprises at least one of FcR bearing, 4-1BBL-transfected APC that can present both the surface-bound molecule (which upregulates 4-1BB [e.g. OKT3]) and an MHC-peptide combination of interest.

In addition, in another embodiment, the present invention relates to 4-1BBL (or part thereof, or another 4-1BB ligand) in conjunction with anti-CD3 (or a specific antigen) for expanding functional human CD4 T cells and human CD8 T cells with cytotoxic activity. In an embodiment, for the human CD8 T cells, this expansion is performed when CD4 and human CD8 T cells are present in the same culture. In a particular embodiment, the CD4 cells are CD28⁺ and CD8 cells are CD28⁻ cells in which a cooperation between the different T cell subtypes further increases the activation.

A new method for the stimulation of cytotoxic T cells in vitro has been developed. This method has implications for the design of vaccination strategies in vivo.

A cell line expressing the MHC molecule of interest is transfected with the human T cell stimulatory molecule, 4-1BB Ligand (CD137 or 4-1BBL ; also see U.S. Pat. No. 6,355,779B). As proof of concept, the murine mastocytoma cell line, P815 was transfected with the gene for human 4-1BBL.

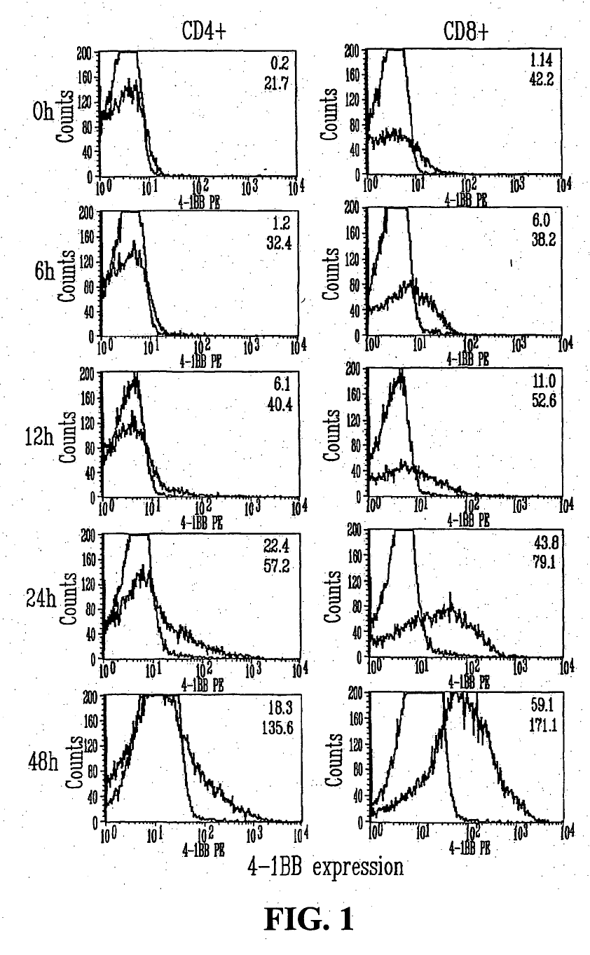

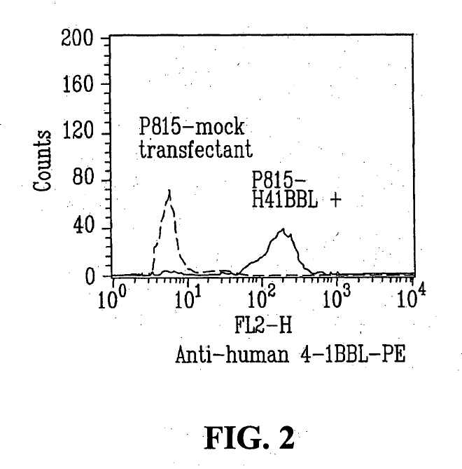

This cell line also expresses Fc receptors and therefore can bind stimulatory anti-CD3 antibodies and can be used to deliver both a TCR signal and an additional “costimulatory” signal from 4-1BBL (FIGS. 1 and 2).

To test the potential of 4-1BBL in augmenting human T cell responses, T cells were isolated from peripheral blood from healthy donors and incubated with the cells expressing 4-1BBL. After incubation with 4-1BBL expressing cells together with anti-human CD3, the T cells were tested for their ability to kill target cells. The human T cells had developed cytotoxic T cell activity that was specific for the MHC ‘type’ expressed by the stimulatory cell (the P815 mastocytoma) and this activity no longer required the 4-1BBL or anti-CD3 molecule. Thus, this method could be used to activate human T cells ex vivo against tumor antigens for reinfusion into patients. In addition, these results also suggest that delivering 4-1BBL to tumors or antigen presenting cells as part of tumor vaccines in vivo could be a useful means of activating an anti-tumor CTL responses.

Thus, in an aspect, the present invention offers a means of activating cytotoxic T cells in vitro. It provides the first direct evidence that 4-1BBL augments the development of effector function (lytic activity) by human cytotoxic T cells. Transfection of 4-1BBL or delivery thereof by virus infection or otherwise into other human cells/tumors and testing of the ability of 4-1BBL in augmenting MHC-restricted anti-tumor responses can thus be carried out.

In an embodiment, the method of the invention uses a non-specific signal (e.g. anti-CD3) to first activate the T cells, after which time, MHC-specific responses develop in the culture in a 4-1BBL-dependent way. Of course, the present invention also covers a specific activation of T cells through the presentation of a chosen antigen (see below).

The present invention also teaches that resting T cells can be activated upon a two-part treatment thereof which comprises an incubation of the resting T cells with a T cell activating amount of anti-TCR (or antigen) and a costimlator (e.g. 4-1BBL, a B7 molecule, or both). In one embodiment of the present invention, the anti-TCR and costimulator (e.g. 4-1BBL) are added substantially simultaneously. In another embodiment, the resting T cells are first pre-activated with anti-TCR and the costimulator (e.g. 4-1BBL) is added after.

In a particular embodiment, 4-1BBL is added within 24 hrs following pre-activation of the resting cells with anti-TCR.

This invention also relates to the fact that a costimulator (e.g. 4-1BB) delivered to human blood adherent cells using for example an adenovirus vector can augment anti-viral immunity. Indeed, it is shown herein that as measured following 4-1BBL delivery to human blood adherent cells, an increased interferon gamma production in response to a challenge with peptides derived from EBV or influenza was observed. These results additionally demonstrate antigen specificity, anti-viral response as well as an additional mode of delivery that would be suitable for in vivo as well as ex vivo therapy.

The present invention also relates to the demonstration that CD28⁻ T cells can respond to a specific costimulatory signal. The potential to further activate CD28⁻ T cells using 4-1BBL (and/or B7, in cases where the population of T cells comprises some [even if a minor proportion] CD28⁺ T cells), is relevant to diseases such as HIV where the majority of memory T cells with anti-viral specificity are found in the CD28⁻ T cell subset. The data presented herein show that 4-1BBL can promote release of IFN-γ from CD28⁻ CD4⁺ and CD8⁺ T cells. IFN-γ is an important cytokine in anti-viral immunity as well as in immunity to intracellular bacteria, for example tuberculosis. HIV patients have greatly diminished acquired immune responses. Thus, these data provide additional evidence for the therapeutic potential of 4-1BBL and methods and compositions of the present invention. Furthermore, the data suggest that in autoimmune diseases such as RA, in which CD28⁻ T cells accumulate to a greater extent, the ability of 4-1BBL to activate the expanded subset of CD4⁺ CD28⁻ T cells might be harmful and strongly suggests that interfering with this pathway would be useful in lowering the chronic inflammation associated with the auto-immune activity in these patients.

The present invention also relates to a composition of matter comprising a vector which harbors 4-1BBL or B7 (or both) sequences according to the present invention and a chosen antigen sequence. In a particular embodiment, the composition of matter comprises an antigen presenting cell which expresses a nucleic acid sequence encoding a specific antigen determinant or presents such antigenic determinant and 4-1BBL or B7 (or both) sequences according to the present invention.

In embodiments, various molecules (e.g. an anti CD-3 antibody such as OKT-3 [see U.S. Pat. No. 4,658,019 of Kung et al., Apr. 14, 1987]) which bind to the T cell receptor (TCR) can be used in accordance with the present invention to enhance the ability of the immune system to respond to an antigen (e.g. immunopotentiation). Non-limiting examples of such molecules include, broadly, molecules which bind the TCR and trigger an upregulation of 4-1BB and particularly monoclonal antibodies against a variable or constant epitope on the cell surface of T cells. Such molecules include MHC/Ag, antibodies to TCR (anti-TCR), bacterial toxins (e.g. staphylococcal enterotoxin B, A, C1, C2, D, E) that engage TCR, MHC/peptide oligomers, bacterial super antigens, and the like. As alluded above and as exemplified below, specific antigens can also be used as a costimulus. In embodiments, such antigens may include viral antigens, e.g. virus particles or parts thereof of viral proteins or peptides or fragments thereof. In embodiments, the virus is a retrovirus, in a further embodiment, HIV, thus in embodiments the viral proteins or peptides may be HIV-derived proteins and peptides.

While the present invention has been exemplified with 4-1BBL as a ligand which binds specifically to 4-1BB, other ligands of 4-1BB could also be used. In embodiments, such a ligand should stimulate both CD4 and CD8 cells. Non-limiting examples of 4-1BB ligands include fragments or variants of human 4-1BBL as well as primate homologs thereof, peptidomimetics thereof and the like, which retain their binding activity to human 4-1BB. A cross-reactivity of anti-4-1BB antibody between human and primate has been reported (http://research.bidmc.harvard.edu/v_path/pathogen_info.asp?p athogen=CD137&species=1&monkeys=rhesus,cynomolgus,pigtailed, chinese). Thus, the present invention also covers the use of primate 4-1BB and 4-1BBL. In an embodiment, such 4-1BB ligands, homologs or peptidomimetics further retain their ability in stimulating both CD4 and CD8 T cells.

As will be recognized by a person of ordinary skill to which the present invention pertains, the present invention also finds utility for diseases and conditions in which a deactivation of T cells could be desired (e. g. inflammatory diseases) as well as to diseases in which one wants to activate T cells (e.g. conditions associated with immunoimpairment). Non-limiting examples of such diseases include all types of infectious diseases and neoplastic diseases and more particularly chronic viral or bacterial infections and cancer. One of the causes of cancer or tumor growth and malignancy is believed to be due to an escaping of the cancer or tumor cell from the immune system, which fails to properly respond to the cancer antigen.

Broadly, the present invention relates to any type of disease in which a modulation of T cell activation is expected to provide a benefit. The present invention finds utility for any conditions or disease state which correlates with a de-activation of 4-1BB or 4-1BBL (or alternatively an activation of 4-1BB or 4-1BBL). More particularly, the present invention finds utility in diseases or conditions which show expanded CD28⁻ T cell populations. For example, in rheumatoid arthritis, the loss of expression of CD28 on the CD4 T cell pool is associated with the severity of the disease (Schmidt, 1996; Martens, 1997). Increased numbers of CD28⁻ T cells are also observed in other auto-immune conditions including systemic lupus erythromatosus and multiple sclerosis (63, 64). These diseases are characterized by chronic immune activation. Without being limited to a particular theory, 4-1BBL may play a role in sustaining this chronic inflammatory condition. The data presented here show that CD28⁻ T cells can respond to 4-1BBL-mediated costimulation by secreting inflammatory cytokines (IFN-γ and TNF-α). Therefore, blocking 4-1BB/4-1BBL interaction using blocking antibodies or soluble forms of the receptor or ligand, or a reduced expression of 4-1BBL, for example, would thus be a suitable immunotherapy of autoimmune diseases where the increased CD28⁻ T cell population contributes to immune pathology.

There has been an extensive amount of published experimental data on mouse 4-1BBL and its role in cytotoxic T cell activation. In view of the complexity of the T cell activation mechanism, and of the significant differences between T cell activation in human vs. mice, there was, until the present invention, no teaching that human and mouse T cells would behave simliarly. For example, the sequences of mouse and human 4-1BB are only 60% identical at the amino acid level. Moreover, the ligands are even less similar, showing 36% identity at the amino acid level (22). Furthermore, as observed in the instant inventors' laboratory, the ligands and receptors do not interact across these species since mouse 4-1BBL could not stimulate T cells through human 4-1BB. It is noteworthy that the cytoplasmic tail of human and mouse 4-1BB is responsible for the transduction of signals upon 4-1BB ligation. This region of the molecule diverges between species. Mouse 4-1BB has a putative lck binding site that is mutated in human 4-1BB and the single tyrosine residue in the cytoplasmic domain of 4-1BB is found at position 220 of human 4-1BB and position 254 of murine 4-1BB.

Prior to the results described herein which show that human 4-1BBL can activate human CD4 and CD8 T cells, there was only limited information that transfected ligand or antibodies to 4-1BB could stimulate human T cells. For example, 4-1BBL-transfected CV1 cells could augment PHA-stimulated or CD3-stimulated T cell proliferation. However other outcomes such as expansion of the T cells over time, production of cytokines or development of killing function were not tested (22). For a human Th1 clone, the effects of anti-4-1BB were only observed in conjunction with CD28 signaling, and 4-1BB was found to enhance proliferation and cytokine production by the anti-CD3 and anti-CD28 stimulated cells (37). Thus, the instant invention provides the first evidence that human 4-1BBL can stimulate both CD4 and CD8 human T cells. In addition, it provides the first evidence that the combination of CD4 T cells and CD8 T cells in the same culture gives a better response to human 4-1BBL and provides the first evidence that human 4-1BBL can augment the development of CD8 T cell killing function. In addition, the cytokine profile obtained with human 4-1BBL is different from that observed in mice, as such the nature of the response in mice as compared to humans shows the differences between these two systems. CD28⁻ T cells from mice produce IL-2 when stimulated with 4-1BBL and anti-CD3, whereas the human CD28⁻ T cells are clearly different. They do not make any detectable IL-2, but make IFN-γ and TNF-α. Similarly, 4-1BBL stimulates IL-4 production by mouse T cells whereas there was no IL-4 detected in the human experiments performed herein.

Another noteworthy aspect of the present invention, is that 4-1BBL works in the apparent absence of CD28. The present invention clearly establishes this point by using purified CD28⁻ T cells and shows that isolated CD4 and CD8 CD28⁻ T cells can respond to 4-1BBL signaling. Although mouse 4-1BBL had been shown to stimulate mouse CD28⁻ T cells, mice do not normally have a CD28⁻ T cell subset. The mouse cells lacking CD28 were generated by gene targeting. In contrast, CD28⁻ T cells arise spontaneously over time in humans (and primates) and are thought to represent a population of memory T cells. They are found in increased numbers in individuals who have had a large immune response due to infection with certain viruses or due to autoimmune conditions or cancer. For this reason they are thought to be a specialized subset of memory cells. Most of the literature suggests that they are more terminally differentiated than CD28⁺ T cells. Thus it was not predictable that because genetically engineered mouse CD28⁻ T cells, and hence somewhat an artificial system, responded to mouse 4-1BBL, that human 4-1BBL would be able to activate the distinct natural human CD28⁻ T cell subset. Prior to the present invention, it was not known whether CD28⁻ T cells were senescent effector cells or memory cells and whether they could be targeted so as to enable a vaccine strategy.

The present invention also establishes for the first time that 4-1BBL allows an upregulation of the survival of T cells by increasing the expression of survival factor Bcl-X_{L} on human CD28⁻ as well as CD28⁺ T cells. Bcl-X_{L} is associated with cell survival as it protects mitochondria against apoptosis. Previous work had shown that CD28 regulates Bcl-X_{L} expression. Although TNFR family members were known to regulate NF-κB, which in turn had been shown to regulate Bcl-X_{L}, there was no evidence prior to the present invention that 4-1BBL could regulate cell survival in a primate in the absence of a CD28 signal. This finding is of importance because it also shows that activated T cells survive, which is of relevance since under viral responses, an activation of T cells is accompanied by a significant proportion of T cell apoptosis. The present invention thus provides direct evidence that 4-1BBL can upregulate the cell survival pathway in humans. A regulation of Bcl-X L expression had been identified by RT-PCR, in human cells, in the presence of anti-CD3, anti-CD28 and 4-1BBL. In these experiments, anti-CD3 plus anti-CD28 and 4-1BBL were provided to the cells at the same time and a measurement of Bcl-X_{L} by RT PCR was carried-out. Thus, the authors did not show protein expression and did not show an effect of 4-1BBL in the absence of anti-CD28(69).

Further, prior to the present invention, it was not known that human CD28⁻ T cells could be activated so as to enhance their cytotoxic capabilities. Indeed, as exemplified herein, CD28⁻ T cells show redirected lysis of targets using a chromium release assay, as well as an increase in perforin levels.

In addition, the present invention relates to the demonstration that delivery or expression of 4-1BBL in chosen cells together with the presentation of a chosen antigen can augment immunity towards this chosen antigen, thereby enabling the therapeutic means.

Unless defined otherwise, the scientific and technological terms and nomenclature used herein have the same meaning as commonly understood by a person of ordinary skill to which this invention pertains. Generally, the procedures for cell cultures, transfection, molecular biology methods and the like, antibody purification and the like, are common methods used in the art. Such standard techniques can be found in reference manuals such as for example Sambrook et al. (1989, Molecular Cloning-A Laboratory Manual, Cold Spring Harbor Laboratories), Ausubel et al. (1994, Current Protocols in Molecular Biology, Wiley, New York), Campbell 1984, in “Monoclonal Antibody Technology: Laboratory Techniques in Biochemistry and Molecular Biology”, Elsevier Science Publisher, Amsterdam, The Netherlands), in Harlow et al. 1988 (in: Antibody-A Laboratory Manual, CSH Laboratories), Klein 1982 (in: Immunology: The Science of Self-Nonself Discrimination, Wiley & Sons, N.Y.), in Immunology Today 10: 254 (1989), and in Kanoff, M. E. 1991: Immunological studies in humans. In Current protocols in Immunology, Vol. 1. Wiley & Sons, New York, p. 7.1.

In order to provide a clear and consistent understanding of terms used in the present description, a number of definitions are provided herein below.

Nucleotide sequences are presented herein by single strand, in the 5′ to 3′ direction, from left to right, using the one letter nucleotide symbols as commonly used in the art and in accordance with the recommendations of the IUPAC-IUB Biochemical Nomenclature Commission.

As used herein, the term “activation” refers to any change induced in the basal or resting state of T cells. Non-limiting examples of such changes include any increase in at least one of the following: cell proliferation, cell division, cytokine production (IFN, TNF enhanced response to an antigen or MHC), DNA synthesis, lymphokine, cytokine reduction, cytotoxic activity, intracellular rise in calcium, increased expression of receptors (e. g. IL2-receptor). While the present invention demonstrates means to activate T cells, support can be found in the application for further activating T cells which are already partly activated (as opposed to resting T cells).

The term “immunopotentiation” as used herein refers to an enhanced ability of the immune system to respond to an antigen.

“Monocyte-related cell” as used herein refers to any cells of monocyte lineage or precursors thereof, such as macrophages and dendritic cells.

The term “cytokines” as used herein refers to a diverse group of soluble proteins which are released by one cell type to mediate a biological effect in a second cell type. Biological effects are varied and include cell proliferation, differentiation, growth. Non-limiting examples of cytokines include interleukins (IL1-12), interferons (IFNα, β and γ) tumor necrosis factor (TNF α, β and the like). The biological effect of a cytokine is generally mediated by the binding thereof to its receptor. The cytokine is often referred as a “ligand” of a receptor. The term “ligand” is well-known in the art of immunology and other ligands include, for example, antibodies which bind a receptor. For certainty, the term “ligand” as used herein is used in its broad sense to refer to a molecule which can bind specifically to another one.

One type of TNF receptor is the 4-1BB receptor. The murine 4-1BB receptor has been described in Kwong et al. 1989, Proc. Natl. Acad. Sci. USA 86: 1963; and in U.S. Pat. No. 6,355, 779. The human homolog of 4-1BB is described in U.S. Pat. No. 6,355,779. The sequences for murine and human 4-1BB ligand can also be found in U.S. Pat. No. 6,355,779. While the present invention is exemplified using full length human 4-1BBL, the present invention is not so limited since biologically active fragments and variants of human 4-1BBL and other primate 4-1BBL could also be used in the context of the present invention. For example, variants of human 4-1BBL comprising the extracellular domain thereof and being deleted or mutated in the intracellular domain (e.g. cytoplasmic tail) of 4-1BBL could be used in the context of the present invention. Of course, such 4-1BB ligand derivatives should retain their capability of binding to human 4-1BB or other primate 4-1BB. Other derivatives of 4-1BBL include fusion proteins comprising a fragment which binds to 4-1BB multimeric forms of 4-1BBL (e.g. dimers or trimers which may exhibit an enhanced biological activity in activating T cells according to the present invention) and the like. Another example of variant of primate 4-1BBL would be a variant in which the TM is deleted or mutated and the extracellular domain of this variant is fused to a signal sequence. The variant primate 4-1BBL could be a multimeric variant thereof which enables export and interaction with primate 4-1BB. Examples of signals which could be fused to such variants include heterologous signal sequences to allow export from cells, signals to allow GPI-linkage to the membrane, or sequences that encode for self-assembling protein domains. Generally, the human 4-1BBL protein is considered to be comprised of three regions: a cytoplasmic domain (amino acids 1-25), a transmembrane domain (amino acids 26-48) and an extracellular domain amino acids 49-254) which binds to 4-1BB. For certainty, the terminology “4-1BBL”, “4-1BB”, “B7”, “B7.l” and “B7.2” relate to primate sequences thereof and preferably human sequences.

In embodiments, the 4-1BBL has a sequence as set forth in SEQ ID NO:2 or a sequence substantially identical thereto. In further embodiments, the 4-1BBL is encoded by a sequence capable of encoding SEQ ID NO: 2 (e.g. SEQ ID NO: 1) or a sequence substantially identical thereto.

The term “adjuvant” is used herein in its conventional meaning to relate to agent which improves the immunogenecity of a composition of the present invention.

The terminology “Fc polypeptide” includes native and mutant forms thereof, as well as variants thereof such as truncated Fc polypeptides which retain the hinge region which promotes dimerization.

The terminologies “antigen” and “antigenic determinant” are very well-known in the art. Indeed, the art teaches how to choose particularly antigenic determinants, how to increase the antigenicity of a peptide, molecule or the like, etc. The strength of an antigen is often referred to as the antigenicity or immunogenicity and relates to the property (which is often quantifiable) in eliciting or inducing an immune response.

The present description refers to a number of routinely used recombinant DNA (rDNA) technology terms. Nevertheless, definitions of selected examples of such rDNA terms are provided for clarity and consistency.

As used herein, “nucleic acid molecule”, refers to a polymer of nucleotides. Non-limiting examples thereof include DNA (e.g. genomic DNA, cDNA), RNA molecules (e.g. mRNA) and chimeras thereof. The nucleic acid molecule can be obtained by cloning techniques or can be synthesized. DNA can be double-stranded or single-stranded (coding strand or non-coding strand (antisense).

The term “recombinant DNA” as known in the art refers to a DNA molecule resulting from the joining of DNA segments. This is often referred to as genetic engineering. The same is true for “recombinant nucleic acid”.

The term “DNA segment”, is used herein, to refer to a DNA molecule comprising a linear stretch or sequence of nucleotides. This sequence when read in accordance with the genetic code, can encode a linear stretch or sequence of amino acids which can be referred to as a polypeptide, protein, protein fragment and the like.

The terminology “amplification pair” refers herein to a pair of oligonucleotides (oligos) of the present invention, which are selected to be used together in amplifying a selected nucleic acid sequence by one of a number of types of amplification processes, preferably a polymerase chain reaction. Other types of amplification processes include ligase chain reaction, strand displacement amplification, or nucleic acid sequence-based amplification, as explained in greater detail below. As commonly known in the art, the oligos are designed to bind to a complementary sequence under selected conditions.

The nucleic acid (e.g. DNA, RNA or chimeras thereof) for practicing the present invention may be obtained according to well known methods.

The term “DNA” molecule or sequence (as well as sometimes the term “oligonucleotide”) refers to a molecule comprised generally of the deoxyribonucleotides adenine (A), guanine (G), thymine (T) and/or cytosine (C), often in a double-stranded form, which can comprise or include a “regulatory element”, as the term is defined herein. DNA can be found in linear DNA molecules or fragments, viruses, plasmids, vectors, chromosomes or synthetically derived DNA. As used herein, particular double-stranded DNA sequences may be described according to the normal convention of giving only the sequence in the 5′ to 3′ direction.

“Homology” and “homologous” refers to sequence similarity between two peptides or two nucleic acid molecules. Homology can be determined by comparing each position in the aligned sequences. A degree of homology between nucleic acid or between amino acid sequences is a function of the number of identical or matching nucleotides or amino acids at positions shared by the sequences. As the term is used herein, a nucleic acid sequence is “homologous” to another sequence if the two sequences are substantially identical and the functional activity of the sequences is conserved (as used herein, the term ‘homologous’ does not infer evolutionary relatedness). Two nucleic acid sequences are considered substantially identical if, when optimally aligned (with gaps permitted), they share at least about 50% sequence similarity or identity, or if the sequences share defined functional motifs. In alternative embodiments, sequence similarity in optimally aligned substantially identical sequences may be at least 60%, 70%, 75%, 80%, 85%, 90% or 95%. As used herein, a given percentage of homology between sequences denotes the degree of sequence identity in optimally aligned sequences. An “unrelated” or “non-homologous” sequence shares less than 40% identity, though preferably less than about 25 % identity, with any of SEQ ID NOs 1-10.

Substantially complementary nucleic acids are nucleic acids in which the complement of one molecule is substantially identical to the other molecule. Two nucleic acid or protein sequences are considered substantially identical if, when optimally aligned, they share at least about 70% sequence identity. In alternative embodiments, sequence identity may for example be at least 75%, at least 80%, at least 85%, at least 90%, or at least 95% . Optimal alignment of sequences for comparisons of identity may be conducted using a variety of algorithms, such as the local homology algorithm of Smith and Waterman, 1981, Adv. Appl. Math 2: 482, the homology alignment algorithm of Needleman and Wunsch, 1970, J. Mol. Biol. 48:443, the search for similarity method of Pearson and Lipman, 1988, Proc. Natl. Acad. Sci. USA 85: 2444, and the computerised implementations of these algorithms (such as GAP, BESTFIT, FASTA and TFASTA in the Wisconsin Genetics Software Package, Genetics Computer Group, Madison, Wis., U.S.A.). Sequence identity may also be determined using the BLAST algorithm, described in Altschul et al., 1990, J. Mol. Biol. 215:403-10 (using the published default settings). Software for performing BLAST analysis may be available through the National Center for Biotechnology Information (through the internet at http://www.ncbi.nlm.nih.gov/). The BLAST algorithm involves first identifying high scoring sequence pairs (HSPs) by identifying short words of length W in the query sequence that either match or satisfy some positive-valued threshold score T when aligned with a word of the same length in a database sequence. T is referred to as the neighbourhood word score threshold. Initial neighbourhood word hits act as seeds for initiating searches to find longer HSPs. The word hits are extended in both directions along each sequence for as far as the cumulative alignment score can be increased. Extension of the word hits in each direction is halted when the following parameters are met: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T and X determine the sensitivity and speed of the alignment. The BLAST program may use as defaults a word length (W) of 11, the BLOSUM62 scoring matrix (Henikoff and Henikoff, 1992, Proc. Natl. Acad. Sci. USA 89: 10915-10919) alignments (B) of 50, expectation (E) of 10 (or 1 or 0.1 or 0.01 or 0.001 or 0.0001), M=5, N=4, and a comparison of both strands. One measure of the statistical similarity between two sequences using the BLAST algorithm is the smallest sum probability (P(N)), which provides an indication of the probability by which a match between two nucleotide or amino acid sequences would occur by chance. In alternative embodiments of the invention, nucleotide or amino acid sequences are considered substantially identical if the smallest sum probability in a comparison of the test sequences is less than about 1, preferably less than about 0.1, more preferably less than about 0.01, and most preferably less than about 0.001.

“Nucleic acid hybridization” refers generally to the hybridization of two single-stranded nucleic acid molecules having complementary base sequences, which under appropriate conditions will form a thermodynamically favored double-stranded structure. Examples of hybridization conditions can be found in the two laboratory manuals referred above (Sambrook et al., 1989, supra and Ausubel et al. 1989, supra) and are commonly known in the art. In the case of a hybridization to a nitrocellulose filter, as for example in the well known Southern blotting procedure, a nitrocellulose filter can be incubated overnight at 65° C. with a labeled probe in a solution containing 50% formamide, high salt (5×SSC or 5×SSPE), 5× Denhardt's solution, 1% SDS, and 100 ug/ml denatured carrier DNA (e. g. salmon sperm DNA). The non-specifically binding probe can then be washed off the filter by several washes in 0.2×SSC/0.1% SDS at a temperature which is selected in view of the desired stringency: room temperature (low stringency), 42° C. (moderate stringency) or 65° C. (high stringency). The selected temperature is based on the melting temperature (Tm) of the DNA hybrid. Of course, RNA-DNA hybrids can also be formed and detected. In such cases, the conditions of hybridization and washing can be adapted according to well known methods by the person of ordinary skill. Stringent conditions will be preferably used (Sambrook et al., 1989, supra).

Probes of the invention can be utilized with naturally occurring sugar-phosphate backbones as well as modified backbones including phosphorothioates, dithionates, alkyl phosphonates and a-nucleotides and the like. Modified sugar-phosphate backbones are generally taught by Miller, 1988, Ann. Reports Med. Chem. 23: 295 and Moran et al., 1987, Nucleic Acids Res., 14: 5019. Probes of the invention can be constructed of either ribonucleic acid (RNA) or deoxyribonucleic acid (DNA), and preferably of DNA.

The types of detection methods in which probes can be used include Southern blots (DNA detection), dot or slot blots (DNA, RNA), and Northern blots (RNA detection). Labeled proteins could also be used to detect a particular nucleic acid sequence to which it binds. Other detection methods include kits containing probes on a dipstick setup and the like.

Although the present invention is not specifically dependent on the use of a label for the detection of a particular nucleic acid sequence, such a label might be beneficial, by increasing the sensitivity of the detection. Furthermore, it enables automation. Probes can be labeled according to numerous well known methods (Sambrook et al., 1989, supra). Non-limiting examples of labels include ³H, ¹⁴C, ³²P, and ³⁵S. Non-limiting examples of detectable markers include ligands, fluorophores, chemiluminescent agents, enzymes, and antibodies. Other detectable markers for use with probes, which can enable an increase in sensitivity of the method of the invention, include biotin and radionucleotides. It will become evident to the person of ordinary skill that the choice of a particular label dictates the manner in which it is bound to the probe.

As commonly known, radioactive nucleotides can be incorporated into probes of the invention by several methods. Non-limiting examples thereof include kinasing the 5′ ends of the probes using gamma ³²P ATP and polynucleotide kinase, using the Klenow fragment of Pol I of E. Coli in the presence of radioactive dNTP (e.g. uniformly labeled DNA probe using random oligonucleotide primers in low-melt gels), using the SP6/T7 system to transcribe a DNA segment in the presence of one or more radioactive NTP, and the like.

As used herein, “oligonucleotides” or “oligos” define a molecule having two or more nucleotides (ribo or deoxyribonucleotides). The size of the oligo will be dictated by the particular situation and ultimately on the particular use thereof and adapted accordingly by the person of ordinary skill. An oligonucleotide can be synthesized chemically or derived by cloning according to well known methods. While they are usually in a single-stranded form, they can be in a double-stranded form and even contain a “regulatory region”.

Amplification of a selected, or target, nucleic acid sequence may be carried out by a number of suitable methods. See generally Kwoh et al., 1990, Am. Biotechnol. Lab. 8: 14-25. Numerous amplification techniques have been described and can be readily adapted to suit particular needs of a person of ordinary skill. Non-limiting examples of amplification techniques include polymerase chain reaction (PCR), ligase chain reaction (LCR), strand displacement amplification (SDA), transcription-based amplification, the replicase system and NASBA (Kwoh et al., 1989, Proc. Natl. Acad. Sci. USA 86,1173-1177 ; Lizardi et [AL.,] 1988, BioTechnology 6: 1197-1202; Malek et al., 1994, Methods Mol. Biol., 28: 253-260; and Sambrook et al., 1989, supra). Preferably, amplification will be carried out using PCR.

Polymerase chain reaction (PCR) is carried out in accordance with known techniques. see, e.g. , U.S. Pat. Nos. 4,683,195; 4,683,202; 4,800,159; and 4,965,188, the disclosures of which are incorporated herein by reference. In general, PCR involves, a treatment of a nucleic acid sample (e.g. , in the presence of a heat stable dna polymerase) under hybridizing conditions, with one oligonucleotide primer for each strand of the specific sequence to be detected. An extension product of each primer which is synthesized is complementary to each of the two nucleic acid strands, with the primers sufficiently complementary to each strand of the specific sequence to hybridize therewith. The extension product synthesized from each primer can also serve as a template for further synthesis of extension products using the same primers. Following a sufficient number of rounds of synthesis of extension products, the sample is analyzed to assess whether the sequence or sequences to be detected are present. Detection of the amplified sequence may be carried out by visualization following EtBr staining of the DNA following gel electrophores, or using a detectable label in accordance with known techniques, and the like. For a review on PCR techniques (see PCR Protocols, A Guide to Methods and Amplifications, Michael et al. Eds, Acad. Press, 1990).

Ligase chain reaction (LCR) is carried out in accordance with known techniques (Weiss, 1991, Science 254: 1292). Adaptation of the protocol to meet the desired needs can be carried out by a person of ordinary skill. Strand displacement amplification (SDA) is also carried out in accordance with known techniques or adaptations thereof to meet the particular needs (Walker et al. 1992, Proc. Natl. Acad. Sci. USA 89: 392-396; and ibid., 1992, Nucleic Acids Res. 20: 1691-1696).

As used herein, the term “gene” is well known in the art and relates to a nucleic acid sequence defining a single protein or polypeptide. A “structural gene” defines a DNA sequence which is transcribed into RNA and translated into a protein having a specific amino acid sequence thereby giving rise to a specific polypeptide or protein. It will be readily recognized by the person of ordinary skill, that the nucleic acid sequence of the present invention can be incorporated into anyone of numerous established kit formats which are well known in the art.

A “heterologous” (e.g. a heterologous gene) region of a DNA molecule is a subsegment of DNA within a larger segment that is not found in association therewith in nature. The term “heterologous” can be similarly used to define two polypeptidic segments not joined together in nature. Non-limiting examples of heterologous genes include reporter genes such as luciferase, chloramphenicol acetyl transferase, β-galactosidase and the like which can be juxtaposed or joined to heterologous control regions or to heterologous polypeptides.

The term “vector” is commonly known in the art and defines a plasmid DNA, phage DNA, viral DNA and the like, which can serve as a DNA vehicle into which DNA of the present invention can be cloned.

Numerous types of vectors exist and are well known in the art. In a particular embodiment, the vector is a viral vector which can introduce e.g. a 4-1BBL or B7 molecule in a chosen cell type. In an embodiment the viral vector is an adenoviral vector. In a particular embodiment, the cell type is an antigen presenting cell.

The use a viral (e.g. adenoviral) vector may provide an advantage as it may stimulate a receptor of the innate immune system on a monocyte or monocyte-related cell, thus contributing further to its conversion to an antigen presenting cell.

The term “expression” defines the process by which a gene is transcribed into mRNA (transcription), the mRNA is then being translated (translation) into one polypeptide (or protein) or more.

The terminology n“expression vector” defines a vector or vehicle as described above but designed to enable the expression of an inserted sequence following transformation or transfection into a host. The cloned gene (inserted sequence) is usually placed under the control of control element sequences such as promoter sequences. The placing of a cloned gene under such control sequences is often referred to as being operably linked to control elements or sequences.

Operably linked sequences may also include two segments that are transcribed onto the same RNA transcript. Thus, two sequences, such as a promoter and reporter sequence” are operably linked if transcription commencing in the promoter will produce an RNA transcript of the reporter sequence. In order to be “operably linked” it is not necessary that two sequences be immediately adjacent to one another.

Expression control sequences will vary depending on whether the vector is designed to express the operably linked gene in a prokaryotic or eukaryotic host or both (shuttle vectors) and can additionally contain transcriptional elements such as enhancer elements, termination sequences, tissue-specificity elements, and/or translational initiation and termination sites.

Prokaryotic expressions are useful for the preparation of large quantities of the protein encoded by the DNA sequence of interest. This protein can be purified according to standard protocols that take advantage of the intrinsic properties thereof, such as size and charge (e.g. SDS gel electrophoresis, gel filtration, centrifugation, ion exchange chromatography, etc.). In addition, the protein of interest can be purified via affinity chromatography using polyclonal or monoclonal antibodies or a specific ligand. The purified protein can be used for therapeutic applications. Prokaryotically expressed eukaryotic proteins are often not glycosylated.

The DNA (or RNA) construct can be a vector comprising a promoter that is operably linked to an oligonucleotide sequence of the present invention, which is in turn, operably linked to a heterologous gene, such as the gene for the luciferase reporter molecule. “Promoter” refers to a DNA regulatory region capable of binding directly or indirectly to RNA polymerase in a cell and initiating transcription of a downstream (3′ direction) coding sequence. For purposes of the present invention, the promoter is preferably bound at its 3′ terminus by the transcription initiation site and extends upstream (5′ direction) to include the minimum number of bases or elements necessary to initiate transcription at levels detectable above background. Within the promoter will be found a transcription initiation site (conveniently defined by mapping with S1 nuclease), as well as protein binding domains (consensus sequences) responsible for the binding of RNA polymerase. Eukaryotic promoters will often, but not always, contain “TATA” boxes and “CCAT” boxes. Prokaryotic promoters contain −10 and −35 consensus sequences, which serve to initiate transcription and the transcript products contain Shine-Dalgarno sequences, which serve as ribosome binding sequences during translation initiation. Non-limiting examples of vectors which can be used in accordance with the present invention include adenoviral vectors, poxviral vectors, VSV-derived vectors and retroviral vectors. Such vectors and others are well-known in the art.

As used herein, the designation “functional derivative” denotes, in the context of a functional derivative of a sequence whether a nucleic acid or amino acid sequence, a molecule that retains a biological activity (either function or structural) that is substantially similar to that of the original sequence. In a preferred embodiment of the present invention, the retained biological activity of the functional derivative of 4-1BBL is that of binding to 4-1BB. This functional derivative or equivalent may be a natural derivative or may be prepared synthetically. Such derivatives include amino acid sequences having substitutions, deletions, or additions of one or more amino acids, provided that the biological activity of the protein is conserved. The same applies to derivatives of nucleic acid sequences which can have substitutions, deletions, or additions of one or more nucleotides, provided that the biological activity of the sequence is generally maintained. When relating to a protein sequence, the substituting amino acid generally has chemico-physical properties which are similar to that of the substituted amino acid. The similar chemico-physical properties include, similarities in charge, bulkiness, hydrophobicity, hydrophylicity and the like. The term “functional derivatives” is intended to include “fragments”, “segments”, “variants”, “analogs” or “chemical derivatives” of the subject matter of the present invention.

Thus, the term “variant” refers herein to a protein or nucleic acid molecule which is substantially similar in structure and biological activity to the protein or nucleic acid of the present invention but is not limited to a variant which retains all of the biological activities of the parental protein, for example. For example, a 4-1BBL variant having its cytoplasmic domain deleted or mutated is within the scope of the present invention. Other examples of variants include 4-1BBL polypeptides which comprise, for example, all or part of the extracellular domain of 4-1BBL yet enabling interaction with 4-1BB but deleted for all or a substantial part of the cytoplasmic domain and some or all of the transmembrane region thereof, but engineered so as to be presented to the T cell. In another embodiment of the present invention, the variant of 4-1BBL is deleted or mutated in the cytoplasmic domain thereof, the transmembrane region enabling an anchoring thereof in the membrane. The extracellular domain should, in most embodiments, retain its biological activity in binding to 4-1BB.

The functional derivatives of the present invention can be synthesized chemically or produced through recombinant DNA technology. All these methods are well known in the art.

As used herein, “chemical derivatives” is meant to cover additional chemical moieties not normally part of the subject matter of the invention. Such moieties could affect the physico-chemical characteristic of the derivative (e.g. solubility, absorption, half-life, decrease of toxicity and the like). Such moieties are exemplified in Remington's Pharmaceutical Sciences (1980). Methods of coupling these chemical-physical moieties to a polypeptide or nucleic acid sequence are well known in the art.

The term “allele” defines an alternative form of a gene which occupies a given locus on a chromosome.

As commonly known, a “mutation” is a detectable change in the genetic material which can be transmitted to a daughter cell. As well known, a mutation can be, for example, a detectable change in one or more deoxyribonucleotide. For example, nucleotides can be added, deleted, substituted for, inverted, or transposed to a new position. Spontaneous mutations and experimentally induced mutations exist. A mutant polypeptide can be encoded from this mutant nucleic acid molecule.

As used herein, the term “purified” refers to a molecule having been separated from a cellular component. Thus, for example, a “purified protein” has been purified to a level not found in nature. A “substantially pure” molecule is a molecule that is lacking in most other cellular components.

As used herein, the terms “molecule”, “compound”, or “agent” are used interchangeably and broadly to refer to natural, synthetic or semi-synthetic molecules or compounds. The term “molecule” therefore denotes for example chemicals, macromolecules, cell or tissue extracts (from plants or animals) and the like. Non-limiting examples of molecules include nucleic acid molecules, peptides, antibodies, carbohydrates and pharmaceutical agents. The agents can be selected and screened by a variety of means including random screening, rational selection and by rational design using for example protein or ligand modeling methods such as computer modeling. The terms “rationally selected” or “rationally designed” are meant to define compounds which have been chosen based on the configuration for example of interacting domains of the present invention (4-1BB and 4-1BBL for example). As will be understood by the person of ordinary skill, macromolecules having non-naturally occurring modifications are also within the scope of the term “molecule”. For example, peptidomimetics, well known in the pharmaceutical industry and generally referred to as peptide analogs can be generated by modeling as mentioned above. Similarly, in a preferred embodiment, the polypeptides of the present invention are modified to enhance their stability. It should be understood that in most cases this modification should not alter the biological activity of the interaction domain. The molecules identified in accordance with the teachings of the present invention have a therapeutic value in diseases or conditions in which the physiology or homeostasis of the cell and/or tissue is compromised by a defect in T cell activation. As defined above, the term “ligand” also encompasses molecules such as peptides, antibodies and carbohydrates.

Non limiting examples of such fusion proteins include hemaglutinin fusions and glutathione-s-transferase (GST) fusions and Maltose binding protein (MBP) fusions. In certain embodiments, it might be beneficial to introduce a protease cleavage site between the two polypeptide sequences which have been fused. Such protease cleavage sites between two heterologously fused polypeptides are well known in the art.

In certain embodiments, it might also be beneficial to fuse the interaction domains of the present invention to signal peptide sequences enabling a secretion of the fusion protein from the host cell. Signal peptides from diverse organisms are well known in the art. Bacterial OmpA and yeast Suc2 are two non limiting examples of proteins containing signal sequences. Examples of eukaryotic signal sequences include myelin associated glycoprotein. The myelin associated glycoprotein signal sequence has been successfully used to obtain secretion of the extracellular domain of murine 4-1BBL from eukaryotic cell lines (6). In certain embodiments, it might also be beneficial to introduce a linker (commonly known) between the interaction domain and the heterologous polypeptide portion. Such fusion protein find utility in the assays of the present invention as well as for purification purposes, detection purposes and the like. It would also be possible to introduce fusion proteins capable of spontaneously forming oligomers of the 4-1BBL-fusion protein.

For certainty, the sequences and polypeptides useful to practice the invention include without being limited thereto mutants, homologs, subtypes, alleles and the like. It shall be understood that generally, the primate 4-1BBL and B7 (e.g. B7.1 and B7.2) sequences of the present invention should encode a functional (albeit defective) interaction domain with primate 4-1BB and more particularly human 4-1BB, and primate CD28 and more particularly human CD28, respectively. It will be clear to the person of ordinary skill that whether an interaction domain of the present invention, variant, derivative, or fragment thereof retains its function in binding to its partner can be readily determined by using the teachings and assays of the present invention and the general teachings of the art.

As exemplified herein, the 4-1BB interaction domain of 4-1BBL can be modified, for example by in vitro mutagenesis, to dissect the structure-function relationship thereof and permit a better design and identification of modulating compounds. However, some derivative or analogs having lost their biological function of interacting with their respective interaction partner (4-1BB or 4-1BBL) may still find utility, for example for raising antibodies. Such analogs or derivatives could be used for example to raise antibodies to the interaction domains of the present invention. These antibodies could be used for detection or purification purposes. In addition, these antibodies could also act as competitive or non-competitive inhibitor and be found to be modulators of the 4-1BB-4-1BB ligand interaction.

A host cell or indicator cell has been “transfected” by exogenous or heterologous DNA (e.g. a DNA construct) when such DNA has been introduced inside the cell. The transfecting DNA may or may not be integrated (covalently linked) into chromosomal DNA making up the genome of the cell. In prokaryotes, yeast, and mammalian cells for example, the transfecting DNA may be maintained on a episomal element such as a plasmid. In addition, the cell might have been “infected” using a viral vector. With respect to eukaryotic cells, a stably transfected cell is one in which the transfecting DNA has become integrated into a chromosome so that it is inherited by daughter cells through chromosome replication. This stability is demonstrated by the ability of the eukaryotic cell to establish cell lines or clones comprised of a population of daughter cells containing the transfecting DNA. Transfection methods are well known in the art (Sambrook et al., 1989, supra ; Ausubel et al., 1994 supra). The use of a mammalian cell as indicator can provide the advantage of furnishing an intermediate factor, which permits for example the interaction of two polypeptides which are tested, that might not be present in lower eukaryotes or prokaryotes. Of course, such an advantage might be rendered moot if both polypeptides tested directly interact. It will be understood that extracts from mammalian cells for example could be used in certain embodiments, to compensate for the lack of certain factors. In addition, the use of mammalian indicator cells favors the correct processing and post-translational modifications of the interacting factors (e. g. glycosylation, phosphorylation and the like). For most of the embodiments of the present invention, it will be understood that the cells to be transfected are mammalian cells, preferably primate cells and more preferably human cells (as exemplified below). Of course, other mammalian cells can also be used, as exemplified using the murine P815 cells. It will also be understood that having now shown that human T cells can be activated using 4-1BBL, a person of ordinary skill could, for certain embodiments, use murine or other mammalian cells in which the relevant human (or primate) sequences have been introduced.

In one particular embodiment, the indicator cell co-expresses 4-1BBL and/or a B7 molecule (e.g. B7.1 or B7.2) and a chosen antigenic determinant. In another embodiment, the indicator cell co-expresses 4-1BBL and/or a B7 molecule (e.g. B7.1 or B7.2) and can present a processed peptide having a chosen antigenic determinant. For example, adherent cells from human blood contain antigen presenting cells which naturally express MHC molecules. These can be transfected with 4-1BBL and genes encoding the antigen of interest, or the antigen of interest can be provided in the form of a peptide. Antigen presenting cells known as dendritic cells can also be propagated from human blood by those skilled in the art using appropriate cytokines and other stimuli. Antigenic peptides or other molecules can be chosen in accordance with the present invention to specifically co-stimulate the T cell receptor as well-known in the art. For example, peptides having from about 8 to 30 amino acids in length, preferably from about 8 to 10 for MHC I specific recognition and up to 25 amino acids for MHC II specific recognition can be provided. Alternatively, genes encoding antigenic proteins or entire proteins or longer peptides can be provided to antigen presenting cells and the antigen presenting cell can be allowed to process them into appropriate size fragments. It should be noted that the particular peptide recognized in an immune response (the epitope) can vary with MHC type of the individual and for some diseases have been well established. In other cases, the epitopes can be determined by testing a panel of overlapping peptides comprising the sequence of the proteins of the pathogen and in this way the appropriate epitope may be identified. In a further embodiment, in the absence of knowledge of a specific epitope, one can use the entire antigen and allow the antigen presenting cell to present a mixture of peptides, among which the dominant epitope will be presented.

Nucleic acids may be delive red to c ells in vivo using methods such as direct injection of DNA, receptor-mediated DNA uptake, viral-mediated transfection or non-viral transfection and lipid based transfection, all of which may involve the use of gene therapy vectors. Direct injection has been used to introduce naked DNA into cells in vivo (see e.g., Acsadi et al. (1991) Nature 332:815-818; Wolff et al. (1990) Science 247:1465-1468). A delivery apparatus (e.g., a “gene gun”) for injecting DNA into cells in vivo may be used. Such an apparatus may be commercially available (e.g., from BioRad). Naked DNA may also be introduced into cells by complexing the DNA to a cation, such as polylysine, which is coupled to a ligand for a cell-surface receptor (see for example Wu, G. and Wu, C. H. (1988) J. Biol. Chem. 263:14621; Wilson el al. (1992) J. Biol. Chem. 267:963-967; and U.S. Pat. No. 5,166,320). Binding of the DNA-ligand complex to the receptor may facilitate uptake of the DNA by receptor-mediated endocytosis. A DNA-ligand complex linked to adenovirus capsids which disrupt endosomes, thereby releasing material into the cytoplasm, may be used to avoid degradation of the complex by intracellular lysosomes (see for example Curiel el al. (1991) Proc. Natl. Acad. Sci. USA 88:8850; Cristiano et al. (1993) Proc. Natl. Acad. Sci. USA 90:2122-2126).

Defective retroviruses are well characterized for use as gene therapy vectors (for a review see Miller, A. D. (1990) Blood 76:271). Protocols for producing recombinant retroviruses and for infecting cells in vitro or in vivo with such viruses can be found in Current Protocols in Molecular Biology, Ausubel, F. M. et al. (eds.) Greene Publishing Associates, (1989), Sections 9.10-9.14 and other standard laboratory manuals. Examples of suitable retroviruses include pLJ, PZIP, pWE and pEM which are well known to those skilled in the art. Examples of suitable packaging virus lines include .psi.Crip, .psi.Cre, .psi.2 and .psi.Am. Retroviruses have been used to introduce a variety of genes into many different cell types, including epithelial cells, endothelial cells, lymphocytes, myoblasts, hepatocytes, bone marrow cells, in vitro and/or in vivo (see for example Eglitis, et al. (1985) Science 230:1395-1398; Danos and Mulligan (1988) Proc. Natl. Acad. Sci. USA 85:6460-6464; Wilson et al. (1988) Proc. Natl. Acad. Sci. USA 85:3014-3018; Armentano et al. (1990) Proc. Natl. Acad. Sci. USA 87:6141-6145; Huber et al. (1991) Proc. Natl. Acad. Sci. USA 88:8039-8043; Ferry et al. (1991) Proc. Natl. Acad. Sci. USA 88:8377-8381; Chowdhury et al. (1991) Science 254:1802-1805; van Beusechem et al. (1992) Proc. Natl. Acad. Sci. USA 89:7640-7644; Kay et al. (1992) Human Gene Therapy 3:641-647; Dai et al. (1992) Proc. Natl. Acad. Sci. USA 89:10892-10895; Hwu et al. (1993) J. Immunol. 150:4104-4115; U.S. Pat. No. 4,868,116; U.S. Pat. No. 4,980,286; PCT Application WO 89/07136; PCT Application WO 89/02468; PCT Application WO 89/05345; and PCT Application WO 92/07573).

Adeno-associated virus (AAV) may be used as a gene therapy vector for delivery of DNA for gene therapy purposes. AAV is a naturally occurring defective virus that requires another virus, such as an adenovirus or a herpes virus, as a helper virus for efficient replication and a productive life cycle (Muzyczka et al. Curr. Topics in Micro. and Immunol. (1992) 158:97-129). AAV may be used to integrate DNA into non-dividing cells (see for example Flotte et al. (1992) Am. J. Respir. Cell. Mol. Biol. 7:349-356; Samulski et al. (1989) J. Virol. 63:3822-3828; and McLaughlin et al. (1989) J. Virol. 62:1963-1973). An AAV vector such as that described in Tratschin et al. (1985) Mol. Cell. Biol. 5:3251-3260 may be used to introduce DNA into cells (see for example Hermonat et al. (1984) Proc. Natl. Acad. Sci. USA 81:6466-6470; Tratschin et al. (1985) Mol. Cell. Biol. 4:2072-2081; Wondisford et al. (1988) Mol. Endocrinol. 2:32-39; Tratschin et al. (1984) J. Virol. 51:611-619; and Flotte et al. (1993) J. Biol. Chem. 268:3781-3790). Lentiviral gene therapy vectors may also be adapted for use in the invention.

General methods for gene therapy are known in the art. See for example, U.S. Pat. No. 5,399,346 by Anderson et al. A biocompatible capsule for delivering genetic material is described in PCT Publication WO 95/05452 by Baetge et al. Methods of gene transfer into hematopoietic cells have also previously been reported (see Clapp, D. W., et al., Blood 78: 1132-1139 (1991); Anderson, Science 288:627-9 (2000); and, Cavazzana-Calvo et al., Science 288:669-72 (2000)).

The present invention also provides antisense nucleic acid molecules which can be used for example to decrease or abrogate the expression of the nucleic acid sequences or proteins of the present invention. An antisense nucleic acid molecule according to the present invention refers to a molecule capable of forming a stable duplex or triplex with a portion of its targeted nucleic acid sequence (DNA or RNA). The use of antisense nucleic acid molecules and the design and modification of such molecules is well known in the art as described for example in WO 96/32966, WO 96/11266, WO 94/15646, WO 93/08845 and U.S. Pat. No. 5,593,974. Antisense nucleic acid molecules according to the present invention can be derived from the nucleic acid sequences and modified in accordance to well known methods. For example, some antisense molecules can be designed to be more resistant to degradation to increase their affinity to their targeted sequence, to affect their transport to chosen cell types or cell compartments, and/or to enhance their lipid solubility by using nucleotide analogs and/or substituting chosen chemical fragments thereof, as commonly known in the art. In one embodiment, antisense molecules targeting 4-1BBL and/or a B7 moleculecan be used to decrease or abrogate the expression thereof and diminish or inhibit T cell activation. This inhibition of activation would be advantageous in auto-immune diseases for example.

In a further embodiment, expression of a nucleic acid encoding a polypeptide of interest, or a fragment thereof, may be inhibited or prevented using RNA interference (RNAi) technology, a type of post-transcriptional gene silencing. RNAi may be used to create a pseudo “knockout”, i.e. a system in which the expression of the product encoded by a gene or coding region of interest is reduced, resulting in an overall reduction of the activity of the encoded product in a system. As such, RNAi may be performed to target a nucleic acid of interest or fragment or variant thereof, to in turn reduce its expression and the level of activity of the product which it encodes. Such a system may be used for functional studies of the product, as well as to treat disorders related to the activity of such a product. RNAi is described in for example Hammond et al. (2001), Sharp (2001), Caplen et al. (2001), Sedlak (2000) and published U.S. patent applications 20020173478 (Gewirtz; published Nov. 21, 2002) and 20020132788 (Lewis et al.; published Nov. 7, 2002), all of which are herein incorporated by reference. Reagents and kits for performing RNAi are available commercially from for example Ambion Inc. (Austin, Tex., USA) and New England Biolabs Inc. (Beverly, Mass., USA).

The initial agent for RNAI in some systems is thought to be dsRNA molecule corresponding to a target nucleic acid. The dsRNA is then thought to be cleaved into short interfering RNAs (siRNAs) which are 21-23 nucleotides in length (19-21 bp duplexes, each with 2 nucleotide 3′ overhangs). The enzyme thought to effect this first cleavage step has been referred to as “Dicer” and is categorized as a member of the RNase III family of dsRNA-specific ribonucleases. Alternatively, RNAi may be effected via directly introducing into the cell, or generating within the cell by introducing into the cell a suitable precursor (e.g. vector encoding precursor(s), etc.) of such an siRNA or siRNA-like molecule. An siRNA may then associate with other intracellular components to form an RNA-induced silencing complex (RISC). The RISC thus formed may subsequently target a transcript of interest via base-pairing interactions between its siRNA component and the target transcript by virtue of homology, resulting in the cleavage of the target transcript approximately 12 nucleotides from the 3′ end of the siRNA. Thus the target mRNA is cleaved and the level of protein product it encodes is reduced.

RNAi may be effected by the introduction of suitable in vitro synthesized siRNA or siRNA-like molecules into cells. RNAi may for example be performed using chemically-synthesized RNA (Brown et al., 2002). Alternatively, suitable expression vectors may be used to transcribe such RNA either in vitro or in vivo. In vitro transcription of sense and antisense strands (encoded by sequences present on the same vector or on separate vectors) may be effected using for example T7 RNA polymerase, in which case the vector may comprise a suitable coding sequence operably-linked to a T7 promoter. The in vitro-transcribed RNA may in embodiments be processed (e.g. using E. coli RNase III) in vitro to a size conducive to RNAi. The sense and antisense transcripts are combined to form an RNA duplex which is introduced into a target cell of interest. Other vectors may be used, which express small hairpin RNAs (shRNAs) which can be processed into siRNA-like molecules. Various vector-based methods are described in for example Brummelkamp et al. (2002), Lee et al. (2002), Miyagashi and Taira (2002), Paddison et al. (2002) Paul et al. (2002) Sui et al. (2002) and Yu et al. (2002). Various methods for introducing such vectors into cells, either in vitro or in vivo (e.g. gene therapy) are known in the art.

Accordingly, in an embodiment expression of a nucleic acid encoding a polypeptide of interest, or a fragment thereof, may be inhibited by introducing into or generating within a cell an siRNA or siRNA-like molecule corresponding to a nucleic acid encoding a polypeptide of interest, or a fragment thereof, or to an nucleic acid homologous thereto. “siRNA-like molecule” refers to a nucleic acid molecule similar to an siRNA (e.g. in size and structure) and capable of eliciting siRNA activity, i.e. to effect the RNAi-mediated inhibition of expression. In various embodiments such a method may entail the direct administration of the siRNA or siRNA-like molecule into a cell, or use of the vector-based methods described above. In an embodiment, the siRNA or siRNA-like molecule is less than about 30 nucleotides in length. In a further embodiment, the siRNA or siRNA-like molecule is about 21-23 nucleotides in length. In an embodiment, siRNA or siRNA-like molecule comprises a 19-21 bp duplex portion, each strand having a 2 nucleotide 3′ overhang. In embodiments, the siRNA or siRNA-like molecule is substantially identical to a nucleic acid encoding a polypeptide of interest, or a fragment or variant (or a fragment of a variant) thereof. Such a variant is capable of encoding a protein having activity similar to the polypeptide of interest. In embodiments, the sense strand of the siRNA or siRNA-like molecule is substantially identical to SEQ ID NOs: 1, 3 and 5, or a fragment thereof (RNA having U in place of T residues of the DNA sequence).

In general, techniques for preparing antibodies (including monoclonal antibodies and hybridomas) and for detecting antigens using antibodies are well known in the art (Campbell, 1984, In “Monoclonal Antibody Technology: Laboratory Techniques in Biochemistry and Molecular Biology”, Elsevier Science Publisher, Amsterdam, The Netherlands) and in Harlow et al., 1988 (in: Antibody-A Laboratory Manual, CSH Laboratories). The present invention also provides polyclonal, monoclonal antibodies, or humanized versions thereof, chimeric antibodies and the like which inhibit or neutralize their respective interaction domains and/or are specific thereto.

From the specification and appended claims, the term therapeutic agent should be taken in a broad sense so as to also include a combination of at least two such therapeutic agents. Further, the DNA segments or proteins according to the present-invention can be introduced into individuals in a number of ways. As exemplified herein, peripheral T cells can be isolated from an individual afflicted or at risk of suffering from a disease or condition, transfected with a DNA construct according to the invention and reintroduced to the afflicted individual in a number of ways, including intravenous injection. Alternatively, the DNA construct can be administered directly to the afflicted individual, for example, by injection in the thymus. The DNA construct can also be delivered through a vehicle such as a liposome, which can be designed to be targeted to a specific cell type, and engineered to be administered through different routes. Of course, proteins or peptides can also be administered. A person of ordinary skill can adapt the transfection method, type of cells transfected, type of disease or condition, co-stimulus (general or specific) etc to meet particular needs. While the therapeutic use of the present invention finds its greatest utility for treating the human disease or condition, the invention should not be so limited, as it is intended to apply to any primate displaying the 4-1BBL/4-1BB activation pathway demonstrated herein. In particular, the present invention applies to primates for which there is an ortholog animal model for a human disease or condition (e.g. SIV).

For administration to humans, the prescribing medical professional will ultimately determine the appropriate form and dosage -for a given patient, and this can be expected to vary according to the chosen therapeutic regimen (e.g. DNA construct, protein, cells), the response and condition of the patient as well as the severity of the disease.

Composition within the scope of the present invention should contain the active agent (e.g. fusion protein, peptide, nucleic acid, and molecule, or antigen, or antibody, or APC) in an amount effective to achieve the desired therapeutic T cell activation while avoiding adverse side effects. Typically, the nucleic acids in accordance with the present invention can be administered to mammals (e.g. humans) in doses ranging from 0.005 to 1 mg per kg of body weight per day of the mammal which is treated. Pharmaceutically acceptable preparations and salts of the active agent are within the scope of the present invention and are well known in the art (Remington's Pharmaceutical Science, 16th ed. , Mack ed. ). The invention therefore further provides a composition comprising an active agent and a pharmaceutically acceptable carrier. For the administration of polypeptides, antagonists, agonists and the like, the amount administered should be chosen so as to avoid adverse side effects. The dosage will be adapted by the clinician in accordance with conventional factors such as the extent of the disease and different parameters from the patient. Typically, 0.001 to 50 mg/kg/day will be administered to the mammal. In accordance with one embodiment of the present invention, and as exemplified herein, T cells may be removed from a patient (e.g. cancer patient, or virally affected patient [or susceptible of being infected by a virus]), activating these T cells in accordance with the present invention and re-administering these activated T cells to the patient. Of course, known steps for further cultivating or proliferating these T cells could be carried-out prior to assaying an activated T cell function or re-injecting same into a patient. For example, cytokines or other mitogens or molecules could added to the culture medium.

As used herein “pharmaceutically acceptable carrier” or “excipient” includes any and all solvents, dispersion media, coatings, antibacterial and antifungal agents, isotonic and absorption delaying agents, and the like that are physiologically compatible. In one embodiment, the carrier is suitable for parenteral administration. Alternatively, the carrier can be suitable for intravenous, intraperitoneal, intramuscular, sublingual or oral administration. Pharmaceutically acceptable carriers include sterile aqueous solutions or dispersions and sterile powders for the extemporaneous preparation of sterile injectable solutions or dispersion. The use of such media and agents for pharmaceutically active substances is well known in the art. Except insofar as any conventional media or agent is incompatible with the active compound, use thereof in the pharmaceutical compositions of the invention is contemplated. Supplementary active compounds can also be incorporated into the compositions.

For certainty, while human 4-1BBL and human 4-1BB and human B7 (B7.1 and B7.2) are preferred sequences (nucleic acid and proteins) in accordance with the present invention, the invention should not be so limited. Indeed, in view of the conservation of these genes within the primates and cross-reacting of anti-4-1BB antibody between humans and other primates, sequences from different primate species, could be used in the compositions, methods and assays of the present invention. Non-limiting examples include primate species in which CD28⁻ cells increase with age or under certain pathological conditions.

It is shown herein that human CD4 and CD8 T cells can respond to human 4-1BBL in the apparent absence of CD28 signaling and that 4-1BBL can augment both expansion and effector function of human T cells. The effects of 4-1BB on the CD8 T cell response are most apparent when both CD4 and CD8 T cells are present in the cultures. Thus CD4 and CD8 T cells cooperate in the response to 4-1BBL-mediated costimulation. These studies provide further support for the development of 4-1BBL as an immunotherapeutic for augmenting suboptimal T cell responses, particularly in situations where CD28-mediated costimulation may be limiting or drastically reduced.

Although anti-CD28 and 4-1BBL were found to synergistically activate human T cells, there was evidence for some CD28-independent costimulation by 4-1BBL in unfractionated T cell cultures. A formal demonstration for a direct role for 4-1BBL in stimulation of human CD28⁻ T cells was sought. The CD28⁻ T cell population from healthy donors was sorted and indeed enabled a demonstration that they can be reactivated to expand, survive and show enhanced effector function following costimulation by 4-1BBL.

It is also shown herein that 4-1BBL can be delivered to chosen cells and specifically augment immunity to a chosen antigen. In the example shown in FIG. 13, peptides representing T cell epitopes from influenza virus or Epstein Barr virus (EBV) (Influenza Ml amino acid sequence=GILGVFTL [SEQ ID NO: 7], EBV sequence=GLCTLVAML [SEQ ID NO: 8]) were synthesized and added to cultures of human cells to which adenovirus-4-1BBL had been introduced. This allowed the reactivation of memory T cells specific for these epitopes in the donor. This provides evidence that 4-1BBL can be delivered to antigen presenting cells and augment antigen specific immunity. In this case, the antigens were chosen based on known T cell epitopes in EBV or influenza. Similarly, this approach could be applied to known epitopes in cancer cells, by synthesizing the desired sequence. Alternatively, a gene encoding the same amino acid sequence could be incorporated into recombinant adenovirus vectors, for example, in a bicistronic vector with 4-1BBL and/or B7 to deliver the two components simultaneously ex vivo or in vivo. In another variation, one could incorporate the gene for the intact viral protein containing the desired epitopes, into the recombinant adenovirus (or other vector) with 4-1BBL and/or B7.

Furthermore, one does not need to know the specific epitope since whole killed tumors or viruses could be delivered to antigen presenting cells, such as dendritic cells, together with the recombinant 4-1BBL and/or B7.

Expression of 4-1BB on Human CD4 and CD8 T Cells

Previous reports have indicated that 4-1BB is expressed on human and mouse CD4 and CD8 T cells although the kinetics of this expression was not determined (17). To determine the kinetics of 4-1BB expression on CD4 versus CD8 T cells, peripheral blood mononuclear cells from healthy donors was obtained and depleted of APC by adherence. In the absence of stimulation, there was no detectable expression of 4-1BB on the lymphocytes from 7 healthy donors examined. To assess the induction of 4-1BB upon activation, peripheral blood lymphocytes (PBL) were incubated with immobilized anti-CD3 and the expression of 4-1BB on CD4 versus CD8 CD3 cells was monitored by 3-colour flow cytometry analysis. FIG. 1 shows a representative example for one healthy donor. Table I summarizes the kinetics of induction of 4-1BB for 7 donors. It can be seen that a greater proportion of CD8 T cells upregulate 4-1BB and to higher levels than on CD4 T cells, but that a proportion of both CD4 and CD8 T cells express 4-1BB after anti-CD3 stimulation in all individuals examined. 4-1BB was detectable within 6 hr of anti-CD3 stimulation and reached a maximum by about 48 hr with most donors and where examined, 4-1BB expression was maintained at 72 hr.

TABLE I
Summary of induction OF 4-1BB on CD4 and CD8 T cells

| | | % of cell population that is 4-1BB+ for each | | | | | | |
|---|---|---|---|---|---|---|---|---|
| | | donor | | | | | | |
| | | Donor | Donor | Donor | Donor | Donor | Donor | Donor |
| Cell population | Time | 1 | 2 | 3 | 4 | 5 | 6 | 7 |
| CD4⁺CD3⁺4-1BB⁺ | 0 h | 0.2 | 0.2 | 0.2 | 0.2 | 0.2 | 1.4 | 0.5 |
| | 6 h | 1.2 | 1.8 | 2.1 | nd | nd | nd | nd |
| | 12 h | 6.1 | 10.5 | 5.4 | 19.1 | 15.1 | 5.4 | 10.5 |
| | 24 h | 22.4 | 18.7 | 21.5 | 16.9 | 24.5 | 17.6 | 12.9 |
| | 48 h | 18.3 | 27.7 | 64.3 | 16.6 | 22.7 | 28 | 21.5 |
| | 72 h | nd | nd | nd | 19.1 | nd | 16.4 | nd |
| CD8⁺CD3⁺4-1BB⁺ | 0 h | 1.1 | 2 | 0.8 | 3.1 | 1.7 | 0.4 | 1.3 |
| | 6 h | 6.0 | 3.4 | 6.8 | nd | nd | nd | nd |
| | 12 h | 11 | 22.7 | 18.3 | 37.2 | 18.4 | 22.5 | 23.7 |
| | 24 h | 43.8 | 51.7 | 63.6 | 47.9 | 37 | 33.6 | 24.6 |
| | 48 h | 59.1 | 72.4 | 88.7 | 62.8 | 56 | 56 | 49.8 |
| | 72 h | nd | nd | nd | 64.5 | nd | 56.5 | 72.4 |

Legend to Table I. Peripheral blood lymphocytes were obtained from healthy donors as described in the Examples. Following depletion of adherent cells, lymphocytes were incubated on plates containing plate bound anti-CD3. After the times of 10 incubation indicated in the table, lymphocytes were removed and analyzed by 3-colour flow cytometry for the expression of CD3, CD4 and CD8 versus isotype control. The table indicates % cells expressing 4-1BB above background after gating on the CD3⁺ CD4⁺ or the CD3⁺ CD8⁺ populations. Results are shown for 7 individual donors tested. For three of the donors, repeat experiments with a separate blood sample showed similar results.

Expansion of T Cells in Response to 4-1BBL Mediated Costimulation

Having established that human CD4 and CD8 T cells can express 4-1B rapidly upon anti-CD3 stimulation, the effect of 4-1BBL stimulation on these T cells was determined. To test the role of 4-1BBL in human T cell activation, the full length human 4-1BBL cDNA or vector control into the P815 mastocytoma cell line was transfected. A subclone of the murine mastocytoma P815 was first isolated by limiting dilution to ensure that the parental cell line was homogeneous. A xenogeneic cell line was chosen to minimize the effects of endogenous costimulatory molecules on the results. FIG. 2 shows flow cytometry analysis of P815 transfected with pcDNA3 versus P815 transfected with 4-1BBL.

P815 cells express Fc receptors and can therefore be used to present anti-CD3 to T cells, thus providing a means of providing the anti-TCR signal as well as the costimulatory signal on the same cells. Flow cytometry analysis was used to determine that the mock transfected P815 and 4-1BBL-transfected P815 bind similar levels of FITC-OKT3 (data not shown).

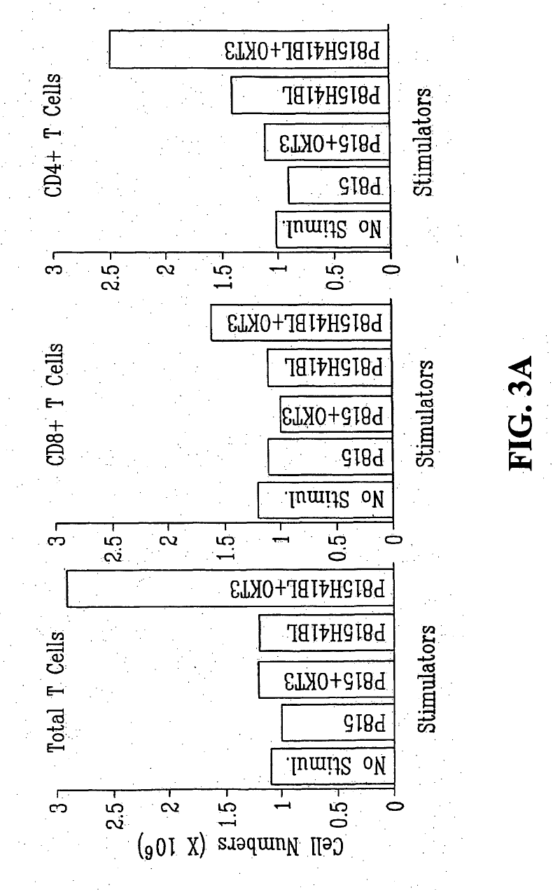

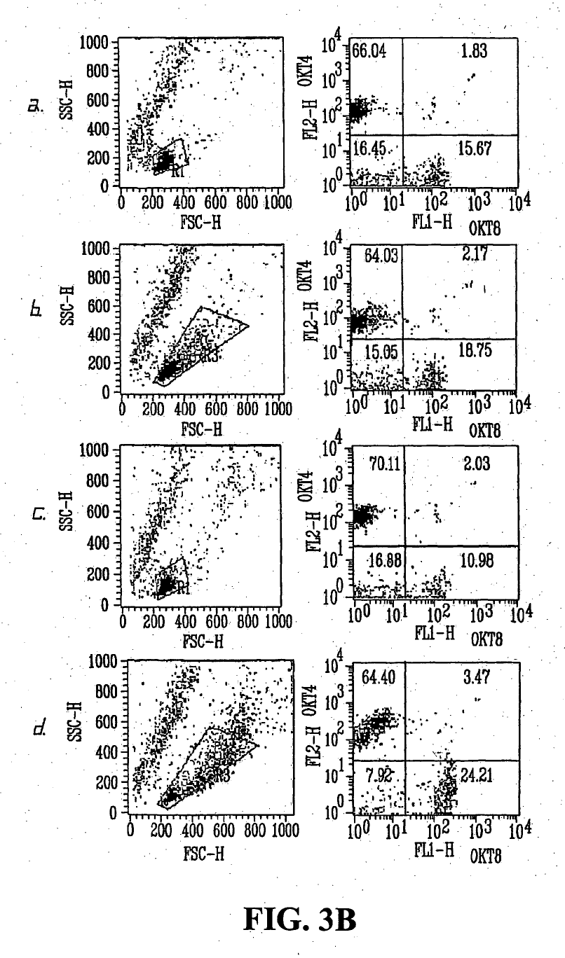

To test the role of 4-1BBL in expansion of total T cells versus CD4 or CD8 T cells, T cells were isolated as described in the Examples and stimulated with P815 cells transfected with vector or 4-1BBL in the presence or absence of OKT3. At the end of the 5-day culture the number of viable cells was determined by trypan blue exclusion and the proportion of CD4 and CD8 T cells analyzed by flow cytometry (FIG. 3). It can be seen that isolated CD4 T cells expand more than the isolated CD8 T cells (FIG. 3A). However, examination of the proportion of the CD4 and CD8 T cells after stimulation of total T cells with P815-4-1BB plus OKT3 indicated that the CD4 and CD8 T cells had both expanded to a similar extent in the total T cell cultures such that the proportion of CD4 and CD8 T cells was not changed in stimulated versus unstimulated cultures (FIG. 3B). Similar results were found with four different donors. Using the total T cell numbers from FIG. 3A and the proportion of CD4 and CD8 T cells in FIG. 3B, both the CD4 and CD8 T cell populations increased by about 2-fold over the five day culture. This expansion reflects the net effects of cell death versus survival and expansion. These results indicate that 4-1BBL can stimulate the survival and/or expansion of both human CD4 and CD8 T cells to similar extent. Thus the presence of CD4 T cells in the culture contributes to the CD8 T cell expansion. It is also apparent from the scatter profiles that the cultures stimulated with both 4-1BBL and OKT3 indicate the presence of enlarged cells in the culture, whereas the unstimulated cultures showed no evidence of the presence of T cell blasts. Cultures stimulated with P815-4-1BB or P815+OKT3 alone showed a much smaller number of enlarged cells in the cultures. It should be noted that attempts to stimulate T cells with 4-1BBL expressed on CHO cells, while providing OKT3 separately were not as efficient. Thus it appears to be preferable, although not necessary, to provide 4-1BBL and anti-TCR general (e.g. OKT3) or specific on the same cell line.

IL-2 Production by CD4 and CD8 T Cells Responding to 4-1BBL-Mediated Costimulation

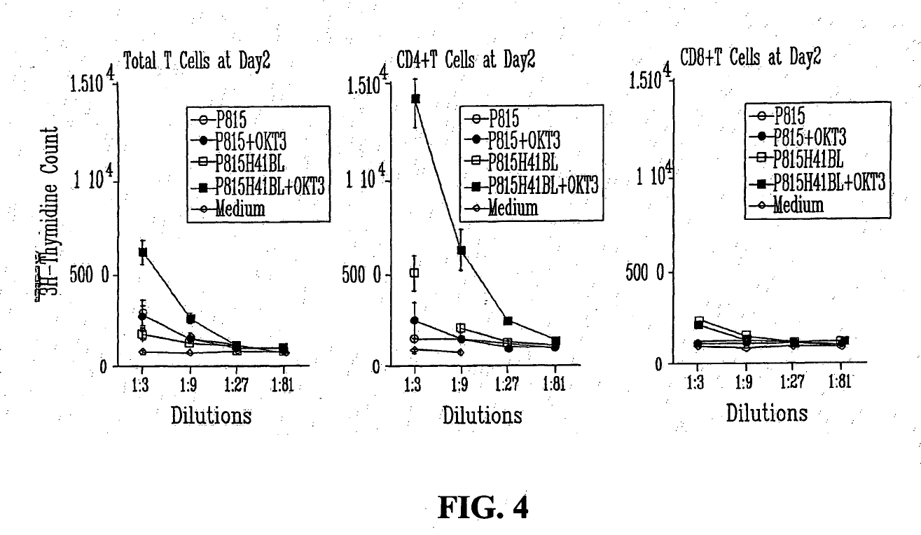

To determine the role of 4-1BBL ON IL-2 production by T cells, unfractionated T cells, or column purified CD4 or CD8 T cells were incubated with P815 cells with or without 4-1BBL and with or without OKT3 as described above. FIG. 4 shows that in the presence of both 4-1BBL and OKT3 there is substantially more IL-2 production in the cultures of total T cells or CD4 T cells than in cultures stimulated with OKT3 alone. In contrast, CD8 T cells produced limited IL-2 in response to 4-1BBL-mediated costimulation. The observation that the CD8 T cells produce no detectable IL-2 in response to 4-1BBL-mediated costimulation provides an explanation for the poorer expansion of the isolated CD8 T cells in FIG. 3. However, in the total T cell cultures, it appears that CD4 T cells can produce IL-2 which in turn can enhance CD8 T cell proliferation.

Augmentation of IFN-γ Production by 4-1BBL

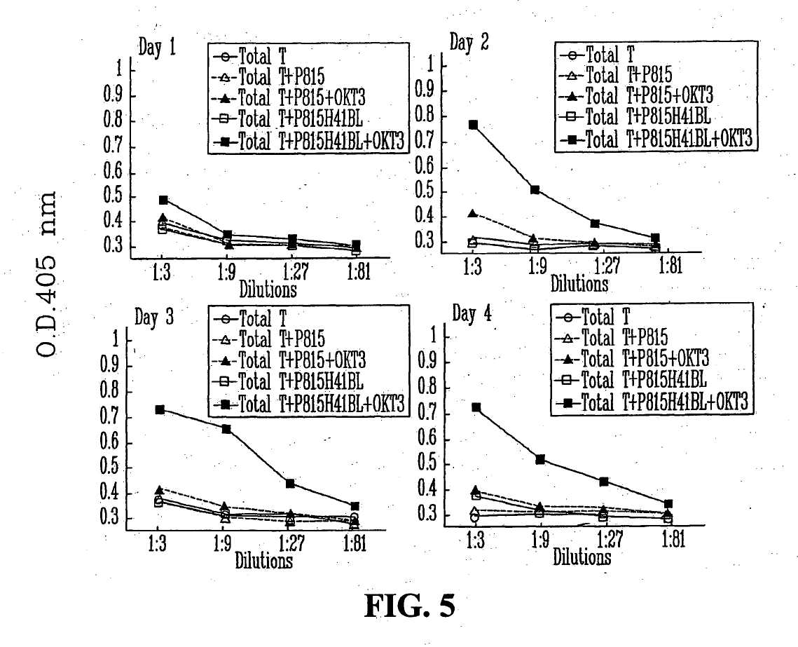

FIG. 5 shows a time course of INF-γ production in cultures of purified human T cells incubated with irradiated P815 cells with or without 4-1BBL and with or without OKT3. It can be seen that the combination of 4-1BBL and OKT3 on the P815 cells allows IFN-γ production by day 2 of culture, whereas 4-1BBL or OKT3 alone do not support IFN-γ production by the T cells. Thus 4-1BBL can provide a costimulatory signal for IFN-γ production by purified human T cells. This finding is consistent with the previous results of Kim et al. (37) who showed that anti-4-1BB could enhance Th1 cytokine production by a Th1 clone responding to anti-CD3 plus anti-CD28.

Role of 4-1BBL in Augmentation of CTL Effector Function

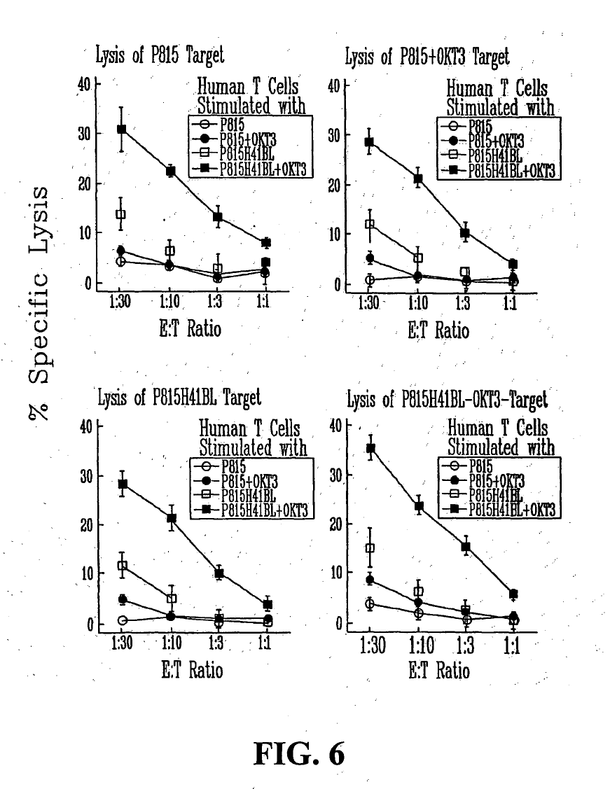

4-1BBL has been shown to augment the development of CTL effector function by mouse CD8 T cells. To test the effect of 4-1BBL on development of human cytotoxic T-lymphocytes (CTL) activity, purified T cells were incubated with P815 cells with and without OKT3 and 4-1BBL as described herein. After 5 days of culture, T cells were tested for their ability to kill ⁵¹Cr-labelled p815 cells. As shown in FIG. 6, the presence of both 4-1BBL and OKT3 on the stimulator cells resulted in a substantial increase in CTL activity against the P815 targets over stimulation with P815-OKT3 or P815-4-1BBL alone, although the presence of 4-1BBL on the P815 cells also showed a small effect on the development of CTL effector function. Once stimulated with 4-1BBL and OKT3, the effector CTL could kill the P815 targets regardless of the presence of anti-CD3 or 4-1BBL (FIG. 6). Thus the CTL appear to have developed a xenoresponse against the P815 cells during the 5 day culture and the development of this response was augmented by 4-1BBL and even more so when 4-1BBL was provided together with OKT3. To further substantiate the conclusion that the T cells have developed a xenogeneic response to P815 cells, CTL effector cells from P815-stimulated cultures were also tested for their ability to kill other targets. It was found that T cells that had been stimulated with P815-4-1BBL plus OKT3 were able to kill the P815 (H-2^{d}) cells and to a lesser extent another (H-2^{d}) target (A20) but were unable to kill an MHC-unrelated target, EL4 (H-2^{b}) or the mouse NK-sensitive target YAC. The response of the T cells to 4-1BBL requires that the T cells receive a signal through the TCR to upregulate 4-1BB. The presence of anti-CD3 in the cultures is expected to result in more effective upregulation of 4-1BB and may enhance the ability of 4-1BBL to augment the development of a xenogeneic response to the P815 target cells.

The data presented herein indicate that human 4-1BBL can provide a costimulatory signal for human T cell activation, thereby allowing T cell expansion as well as cytokine production and the development of CTL effector function. Thus, as previously demonstrated for murine 4-1BBL and surprisingly, human 4-1BBL can function as a costimulatory molecule for CD4 and CD8 T cell activation. For murine T cells, it had been recently reported that isolated CD4 and CD8 T cells expand to a similar extent to anti-CD3 plus 4-1BBL-mediated costimulation (13). Herein, when total T cells were stimulated with 4-1BBL together with OKT3 on the surface of P815 cells, both CD4 and CD8 T cells expanded to a similar extent, consistent with both CD4 and CD8 T cells responding to 4-1BBL-mediated costimulation. However, in cultures of isolated CD8 T cells, the amount of T cell expansion was less (FIG. 3) and this is attributed to the lower levels of IL-2 produced by the CD8 T cells (FIG. 4) as compared to CD4 T cells. These studies demonstrate that 4-1BBL exerts its maximal effect in cultures containing both CD4 and CD8 T cells. The net expansion of the T cells in the P815/OKT3/4-1BBL stimulated cultures was relatively modest (about 2 fold). The recovery of cells in the cultures reflects the net effects of cell death versus cell survival and division. In these cultures there were no human B7 family members on the stimulator cells, so the only source of B7 family members would be on contaminating APC or on activated T cells. Thus, B7 family molecules are likely to be present at most at very low levels in the cultures. Since CD28 B7 interaction is thought to be critical in initial expansion of T cells, the relatively modest expansion of the T cells using primarily 4-1BBL as the primary costimulus is not surprising, but does suggest, that like murine 4-1BBL, human 4-1BBL can promote CD28-independent T cell activation.

4-1BBL was also able to augment the development of CTL effector function (FIG. 6). Once the CTL activity was induced, however, the presence of 4-1BBL on the target cells did not effect the level of lysis. These data imply a role for 4-1BBL in the expansion of the CTL with concomitant development of CTL effector function, but rule out a role for 4-1BB/4-1BB in the actual killing function of the CTL. Although the generation of maximal CTL activity in the cultures required both OKT3 and 4-1BBL on the P815 stimulator CELLS, 4-1BB on P815 alone induced some activity, consistent with the finding that the target cell specificity indicated that a xeno-response has been induced to the P815 cells that becomes independent of CD3 in the cultures by the time of the ⁵¹Cr-release assay. The enhancement of the development of CTL activity due to the presence of OKT3 may be due to the requirement for a strong signal through the TCR to induce 4-1BB expression, a prerequisite for the response to 4-1BBL.

4-1BB was found to be inducible on both CD4 and CD8 T cells as well as on both CD45RO and CD45RA subsets of these cells. CD8 T cells upregulated 4-1BB to a greater and more rapid extent than CD4 T cells, when unfractionated lymphocyte cultures were stimulated with plate bound anti-CD3. In spite of the differences in the level of 4-1BB upregulation by CD4 versus CD8 T cells, the studies in FIG. 3 imply that both CD4 and CD8 T cells respond to 4-1BBL-mediated costimulation in terms of net expansion observed in the cultures.

Taken together, the results presented hereinabove suggest a strategy to augment human MHC-restricted responses using a combination of a signal through the TCR to upregulate 4-1BB rapidly (e.g. OKT3) as well as a FcR bearing, 4-1BBL-transfected APC that can present both the soluble OKT3 and an MHC-peptide combination of interest. In addition, the results suggest that 4-1BBL in conjunction with anti-CD3 provides an effective method for expanding functional CD4 T cells and CD8 T cells with cytotoxic activity. For the CD8 T cells, this expansion is most efficient when both CD4 and CD8 T cells are present in the same culture and independently of CD28. In addition, these findings enable the corollary strategy: for diseases or conditions in which a deactivation of T cells would be advantageous (e.g. auto-immune diseases), a blocking of 4-1BBL function should be pursued. Of note, the OKT3mAb is used currently to prevent renal transplant rejection (Goldstein, Transpl. Proc. 1987 XIX (2), Supp. 1:1).

Although various embodiments of the invention are disclosed herein, many adaptations and modifications may be made within the scope of the invention in accordance with the common general knowledge of those skilled in this art. Such modifications include the substitution of known equivalents for any aspect of the invention in order to achieve the same result in substantially the same way. Numeric ranges are inclusive of the numbers defining the range. In the claims, the word “comprising” is used as an open-ended term, substantially equivalent to the phrase “including, but not limited to”. The following examples are illustrative of various aspects of the invention, and do not limit the broad aspects of the invention as disclosed herein.

Throughout this application, various references are referred to describe more fully the state of the art to which this invention pertains. The disclosures of these references are hereby incorporated by reference into the present disclosure.

### EXAMPLES

The present invention is illustrated in further detail by the following non-limiting examples.

### Example 1

### Cell Lines and Antibodies

The DBA/2 mastocytoma P815, the chemically induced C57BL/6 lymphoma EL4 and the Moloney leukemia virus induced lymphoma of an A/Sn mouse, YAC-1 were obtained from the American Type Culture Collection (ATCC, Rockville, Md.). The IL-2 dependent line CTLL-2 and the human monocytic line THP-1 were also obtained from the ATCC. All cell lines were maintained in complete culture medium (CCM), which was prepared with RPMI-1640 medium (SIGMA, St. Louis Mo.) supplemented with 10% heat-inactivated foetal bovine serum, (Cansera, Rexdale, Ontario, Canada), 50 μM 2-ME, MEM nonessential amino acids (Life Technologies, Gaithersburg, Md.), antibiotics, pyruvate, and glutamine.

Anti-human CD3 OKT3, was purified from culture supernatant of the hybridoma using protein G-Sepharose™ (Pharmacia, Piscataway, N.J.) column, and conjugated to fluorescein using fluorescein isothiocyanate (Molecular Probes). OKT3 was obtained from the American Type Culture Collection (Rockville, Md.). OKT3, OKT4 and FITC or PE conjugated anti-human CD4, CD8 and 4-1BB ligand monoclonal antibodies were purchased from BD PharMingen (San Diego, Calif.).

### Example 2

### Transfection of P815 Cells with Human 4-1BB Ligand Gene

Cytoplasmic RNA for cDNA cloning was prepared from THP-1 monocytic cells using RNeasy™ mini kit (QIAGEN, Germany). Human 4-1BBL cDNA was synthesized with First Strand™ cDNA Syntheses Kit (Boehringer Mannheim, Indianapolis, Ind.), and PCR-amplified with HotStarTaq™ polymerase (QIAGEN, Germany). PCR primers were designed based on the published sequence. The transfection construct was made by insertion of PCR product into the pcDNA3 vector (Invitrogen, Carlsbad, Calif.) at an EcoRI site.

P815 cells were cloned by limiting dilution in 96-well culture plates. An individual clone, confirmed to bind FITC-OKT3 at high levels, was chosen for the transfection. The cloned P815 cells were transfected by electroporation with human 4-1BBL construct and selected with Geneticin (GibcoBRL, Grand Island N.Y.) for neomycin resistance. The resistant cells were sorted for human 4-1BB ligand expression with PE-conjugated anti human 4-1BBL mAb, and a clone with high expression was used as a stimulator/target in the T cell functional assays described below. Cloned P815 cells were also transfected with pcDNA3 vector only (mock transfection), and a neomycin-resistant clone were used as 4-1BBL negative control stimulator/target.

### Example 3

### T Cell Isolation

Human peripheral blood was collected from healthy volunteers after obtaining signed consent according to a protocol approved by the University of Toronto Human Subjects Review Committee. Blood was mixed with an equal volume of phosphate buffered saline (PBS) and overlaid on Ficoll-Paque Plush (Amersham Pharmacia, Uppsala, Sweden), and centrifuged at 300 g for 20 min. Mononuclear cells at the interface were collected, washed twice with PBS and resuspended in CCM. Cells in CCM were incubated in culture flask at 37° C. for one hour to deplete the plastic-adherent macrophages and monocytes. Non-adherent cells were collected in PBS and loaded on isolation columns for total, CD4+ or CD8+ human T cell separation (Cedarlane Laboratories, Hornby, Ontario, Canada).

Purified human T cells were column purified according to the manufacturer's protocol, and resuspended in CCM for functional assays. Similarly, CD45RO or CD45RA cells were purified using columns to deplete the unwanted subsets, also obtained from Cedarlane Laboratories and used according to the manufacturer's instructions.

### Example 4

### T Cell Stimulation Assays

Purified T cells were mixed with the 80 Gy γ-irradiated stimulator cells at 2:1 (E:S) ratio in CCM, and co-cultured with stimulators on either 96-well or 24 well plates for 2 to 5 days. Culture supernatant was collected for cytokine assays and the responder cells were harvested for counting, FACS analysis and analysis of CTL function in a ⁵¹-Chromium release assay. To prepare the OKT3-loaded stimulatory cells, mock or 4-1BBL transfected P815 cells were suspended in PBS at 10⁷ cells/ml, mixed with OKT3 and incubated at 37° C. for one hour. After the incubation, the cells were washed three times with PBS to remove the unbound OKT3. Initial experiments involved titration of the anti-CD3 and it was determined that a starting concentration of 0.25 μg of OKT3 per 10⁶ T cells resulted in minimal stimulation with OKT3 alone while maintaining optimum effect of 4-1BBL on T cell stimulation. Therefore, this concentration of OKT3 was used in the experiments described herein.

### Example 5

### Cytokine Assays

Human IL-2 production was analyzed by measuring ³H-thymidine incorporation of IL-2 dependent CTLL-2 cells as described previously (10). Interferon-γ (IFN-γ) levels in the culture supernatant were measured by ELISA using a cytokine detection kit obtained from eBioscience (San Diego, Calif.) according to the manufacturer's instructions.

### Example 6

### Cytotoxic T Cell Assays

Cytotoxic T cell effector function was measured by a standard ⁵¹Cr release assay. Effectors and targets were co-cultured at 37° C. for 4 hours, and the radioactivity of supernatant determined using a Top Count™ scintillation counter (Canberra-Parkard, Meiden, Conn.).

### Example 7

### Expression of 4-IBB on CD28⁻ Cells and Characterization of the CD28^{'1} T Cell Subset

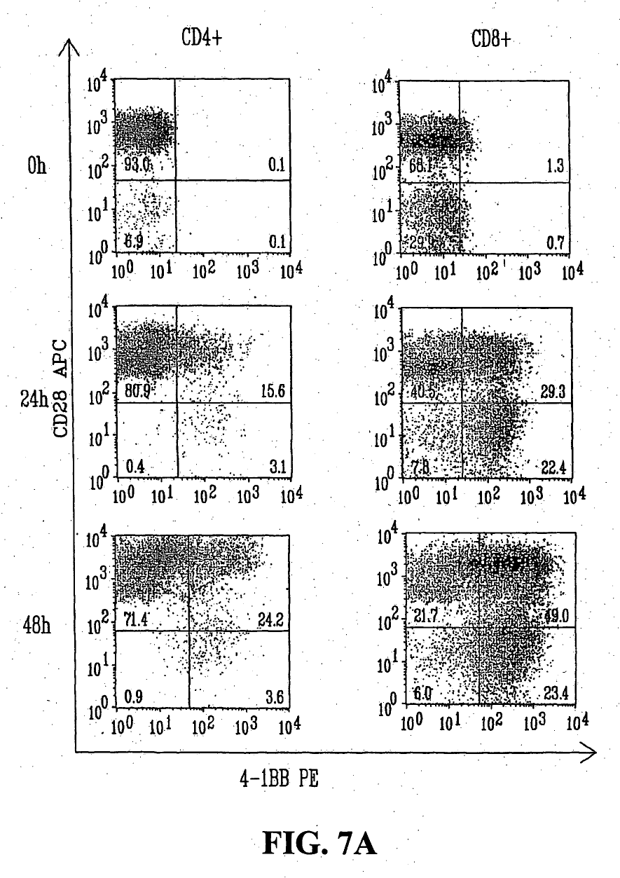

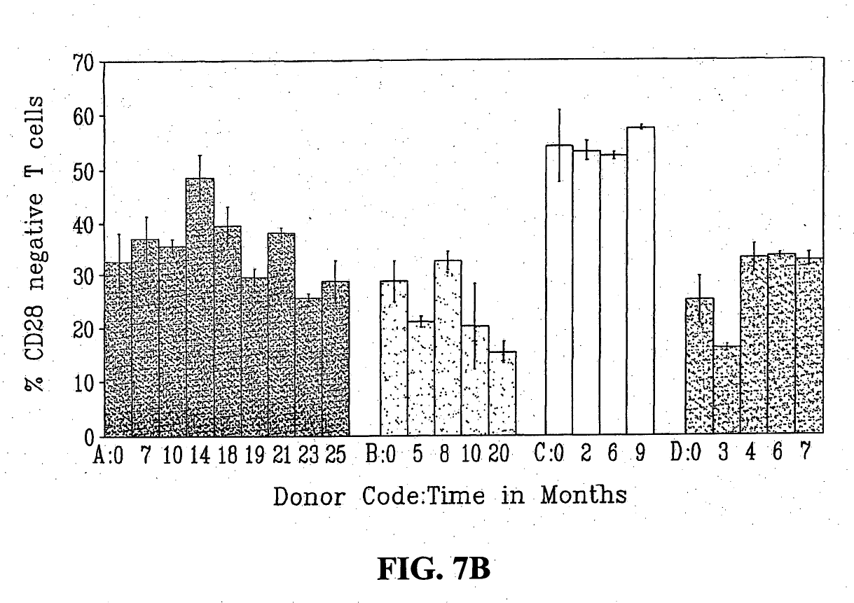

To determine whether CD28⁻ T cells can express 4-1BB after activation, peripheral blood mononuclear cells were obtained from a panel of healthy donors, (age 23-55) and stimulated with plate bound anti-CD3 to induce 4-1BB expression. 4-1BB expression was analyzed on CD4⁺ and CD8⁺, CD28⁺ and CD28⁻ CD3⁺ cells by 4-colour flow cytometry. In most donors, almost all the CD28⁻ T cells became 4-1BB positive after as little as 24 h of stimulation. In contrast, the upregulation of 4-1BB on CD28⁺ T cells proceeded more slowly. This was observed with the CD8⁺ CD28⁻ as well as the CD4⁺ CD28⁻ T cells (FIG. 7a and data not shown). For the 11 donors examined, no detectable expression of 4-1BB was observed in the absence of anti-CD3 stimulation. As demonstrated previously, CD8 T cells expressed higher levels of 4-1BB with faster kinetics of induction than CD4 T cells (58).

The frequency of CD28⁻ T cells in donors ranged from <1% to 55% of the CD3⁺ cells. For further analysis of T cell responses, CD28 negative T cells were isolated from donors with ≧15% CD28⁻ T cells. There was no significant correlation between the percentage of CD28⁻ T cells and the age or sex of the donor over the age range examined. In the majority of donors, the CD8⁺ CD28⁻ population was dominant, often consisting of >95% of CD28⁻ T cells. CD4⁺ CD28⁻ T cells were found in 9 of 11 donors. The proportion of CD28 T cells that were CD4⁺ ranged from 0-46%, with a median of 4.4%. CD4⁺ CD28⁻ T cells also upregulated 4-1BB (FIG. 7a). In all cases, the percentage of CD28⁻ T cells was stable in donors over time, albeit with some fluctuation (FIG. 7b). Freshly isolated CD28⁻ T cells did not express HLA-DR or CD25, implying that they are not activated effectors (data not shown). The CD28⁻ T cell population was heterogeneous for CD45RO and CD45RA expression, but in most donors a larger proportion of CD28⁻ T cells were CD45RA⁺ in comparison to CD28⁺ T cells (data not shown). While CD45RA⁺ expression has been interpreted to imply a naive phenotype, memory T cells can express the CD45RA isoform, a condition associated with the loss of CD28 expression (59).

### Example 8

### Costimulation of Cell Division and T Cell Expansion by 4-1BBL

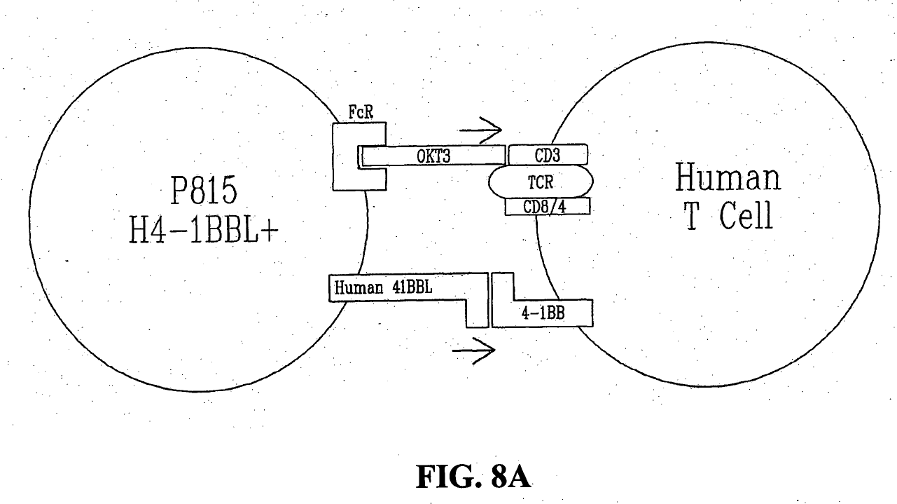

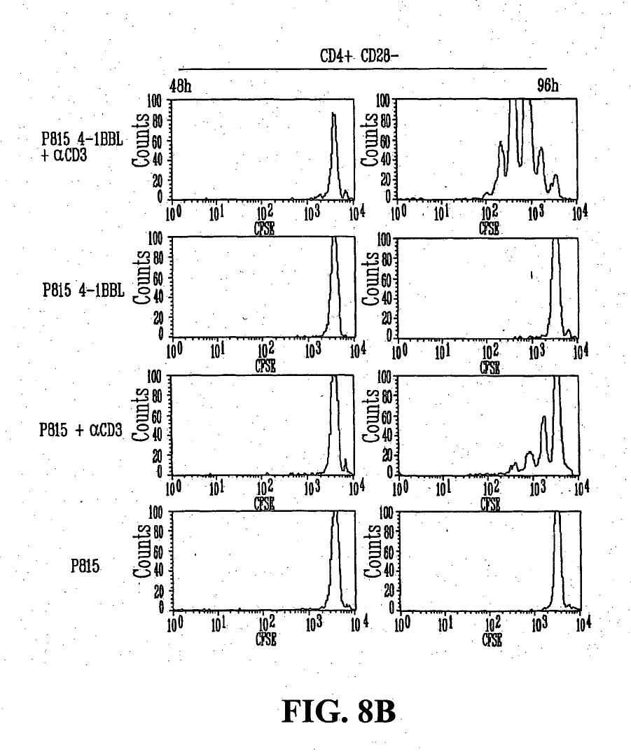

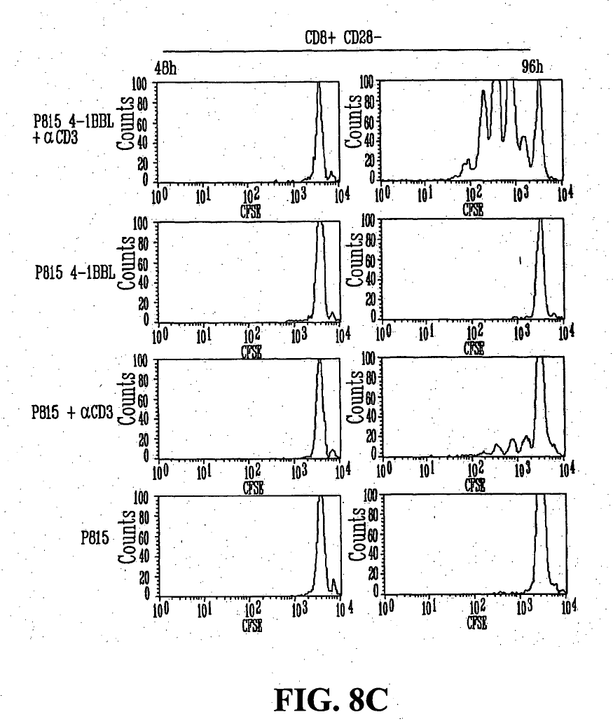

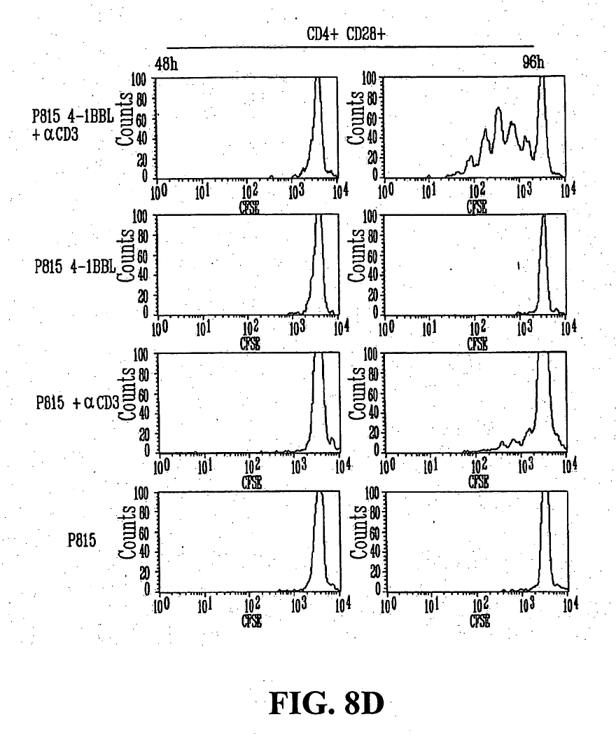

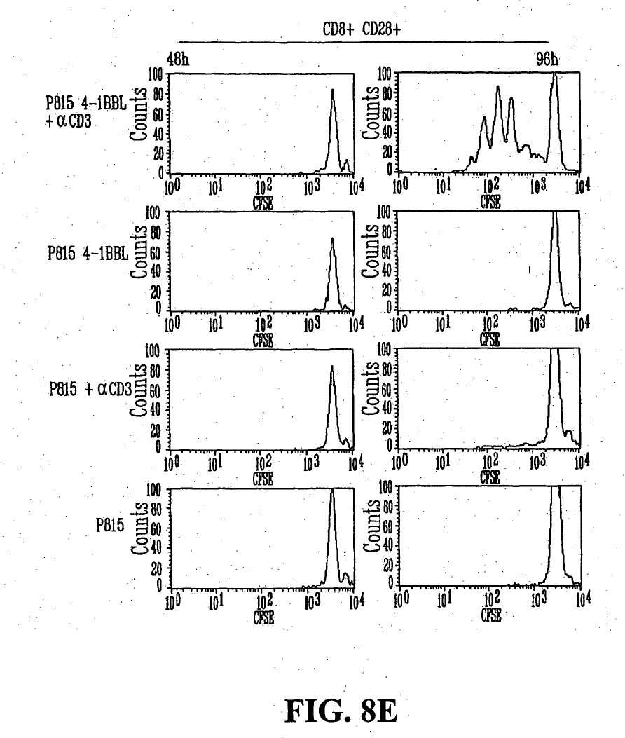

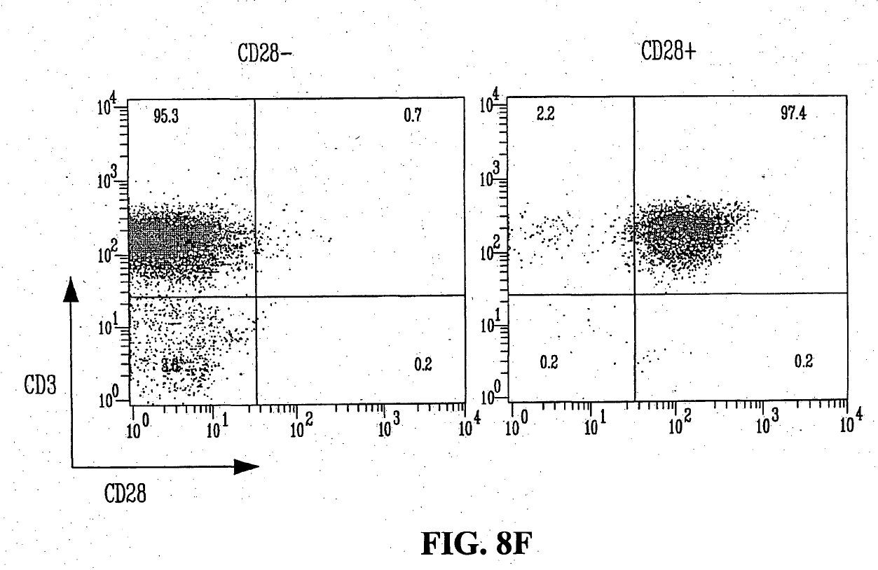

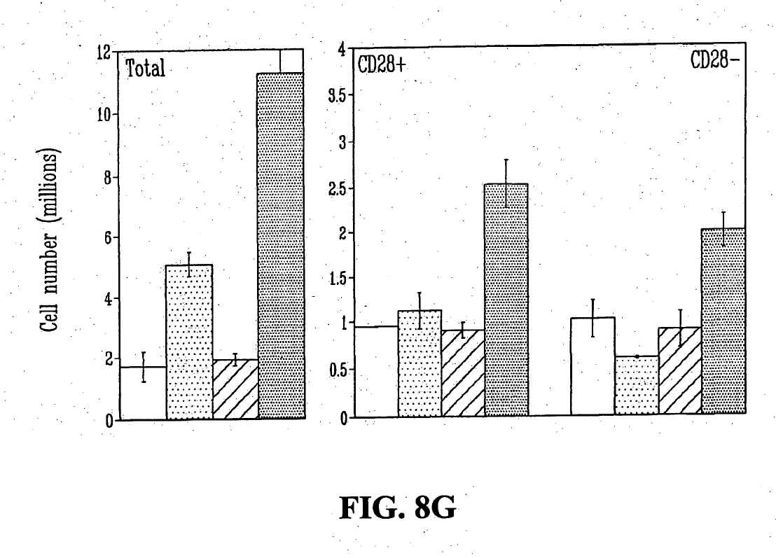

To test the effect of 4-1BBL on human CD28⁺ and CD28⁻ T cells, unfractionated purified T cells were labelled with CFSE and stimulated with P815 cells with or without transfected human 4-1BBL in the presence or absence of anti-CD3 antibodies bound to the FcR of the P815 cells (FIG. 8a). Cells were gated on the CD4⁺ CD28⁺, CD4⁺0 CD28⁻, CD8⁺ CD28⁺ and CD8⁺ CD28⁻ populations and analyzed for cell division. Cell division was first detected in the cultures by 72 hr (not shown) and was more extensive by 96 hr (FIG. 8b). Stimulation with anti-CD3 alone induced a modest amount of cell division by 96 hr of culture, whereas the combination of 4-1BBL and anti-CD3 allowed a substantial proportion of all four T cell subsets to divide (FIG. 8b). In donors with a predominant CD8⁺ CD28⁻ population as well as in a single donor where both CD4⁺ and CD8⁺ CD28⁻ T cells were present in almost equal proportion, each of the populations showed similar rates of cell division in response to anti-CD3 plus 4-1BBL (FIG. 8b and data not shown). The combination of anti-CD3 plus 4-1BBL stimulation induced a greater response than either signal alone with all donors and T cell subsets examined. However, the CD28⁻ T cells consistently showed a small amount of cell division in response to anti-CD3 alone, whereas CD28⁺ T cells were unresponsive to TCR signalling in the absence of costimulation (FIG. 8b). CD45RA⁺ CD28⁻ T cells divided as efficiently as CD45RA⁻ CD28⁻ T cells, with subsequent loss of CD45RA expression (data not shown).

To test the effects of 4-1BBL on expansion of separated CD28⁺ and CD28⁻ T cells, the two populations were isolated by cell sorting. On average, the purity of the T cells was about 95% with less than 1% CD28⁺ T cells contaminating the CD28-population (FIG. 8c). The net expansion of cells in the cultures was followed by counting the number of viable cells recovered after 5 or 6 days of stimulation (FIG. 8d). There was no expansion of isolated CD28⁺ or CD28⁻ T cells in cultures stimulated with either anti-CD3 or 4-1BBL alone, whereas the isolated CD28⁺ and CD28⁻ T cells showed a 2-fold expansion upon stimulation with both anti-CD3 and 4-1BBL. This modest expansion reflects the net effects of expansion versus death over the 5-day culture and clearly shows that 4-1BBL can contribute to expansion and survival of purified CD28⁺ and CD28⁻ T cells.

### Example 9

### IL-2 Production in 4-1BBL-Stimulated Cultures

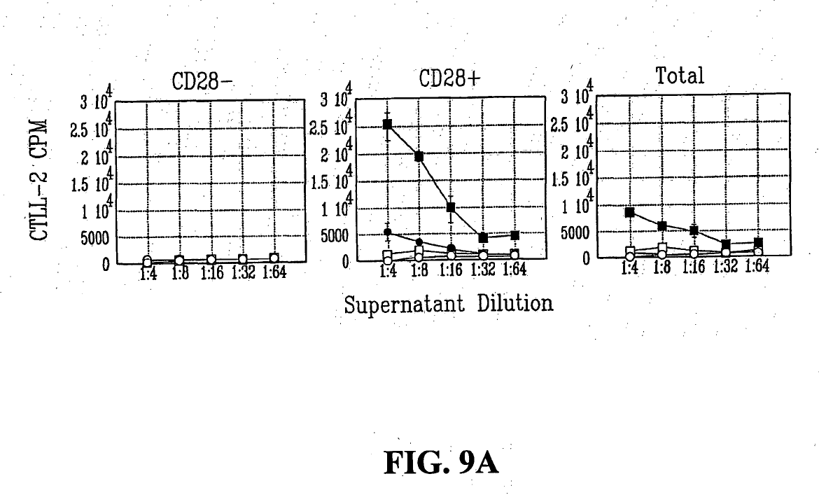

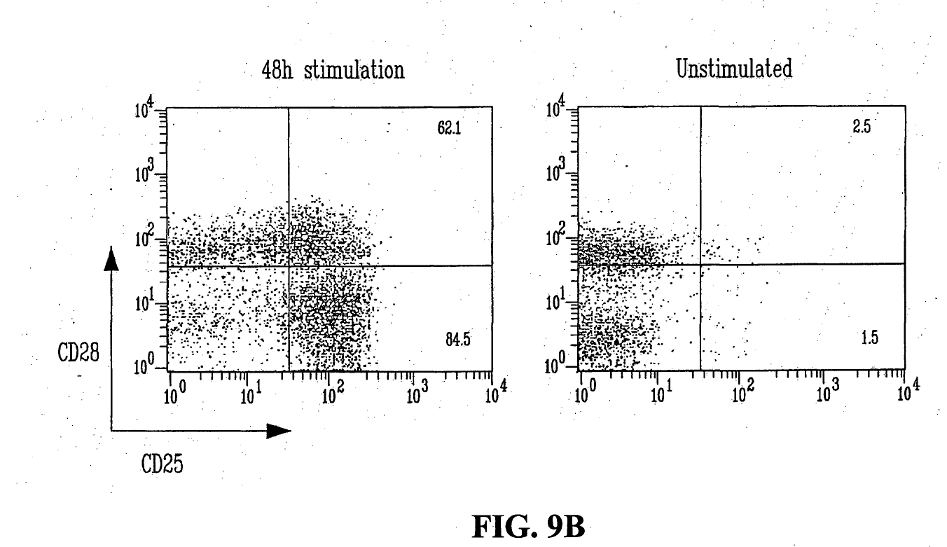

In experiments with several donors, isolated CD28⁻ T cells produced little or no IL-2, whereas sorted CD28⁺ or unfractionated cultures released IL-2 into the supernatant as measured in a bioassay for active IL-2 (FIG. 9a). This was also the case for the donor with a substantial CD4⁺ CD28⁻ T cell population (data not shown).

Although the CD28⁻ T cells did not produce detectable IL-2, they likely respond to IL-2, as evidenced by cell division. Consistent with this, treatment with either anti-CD3 alone or with anti-CD3 plus 4-1BBL induced the expression of the high affinity IL-2Ra chain, CD25, on both the CD28 and CD28⁻ T cell population (FIG. 9b).

### Example 10

### Mechanism of 4-1BBL-Induced T Cell Survival

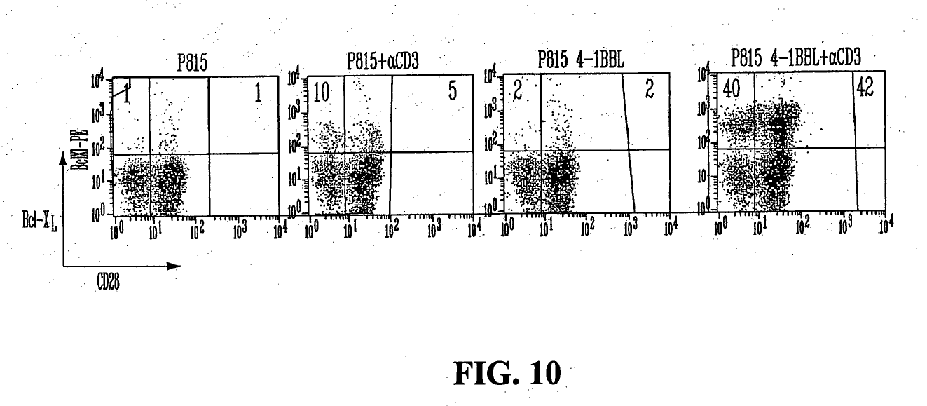

A feature of T cell costimulation method and compositions of the present invention is their ability to induce T cell survival. CD28 signaling promotes T cell survival by regulating expression of the anti-apoptotic protein Bcl-X_{L} (60). It was thus of interest to verify whether the finding that 4-1BBL allows a net expansion of CD28⁻ T cells after 5 days of culture is linked to the fact that the TCR signalling in the presence of a 4-1BBL costimulation (e. g. anti-CD3 plus 4-1BBL signal) not only allows cell division but also promotes T cell survival. It was found that treatment with anti-CD3 PLUS 4-1BBL upregulated Bcl-X_{L} protein over basal levels in both the CD28⁺ and CD28⁻ T cells (FIG. 10).

### Example 11

### Analysis of Effector Cytokine Production by CD28+ and CD28− T Cells Responding to 4-1BBL-Mediated Costimulation

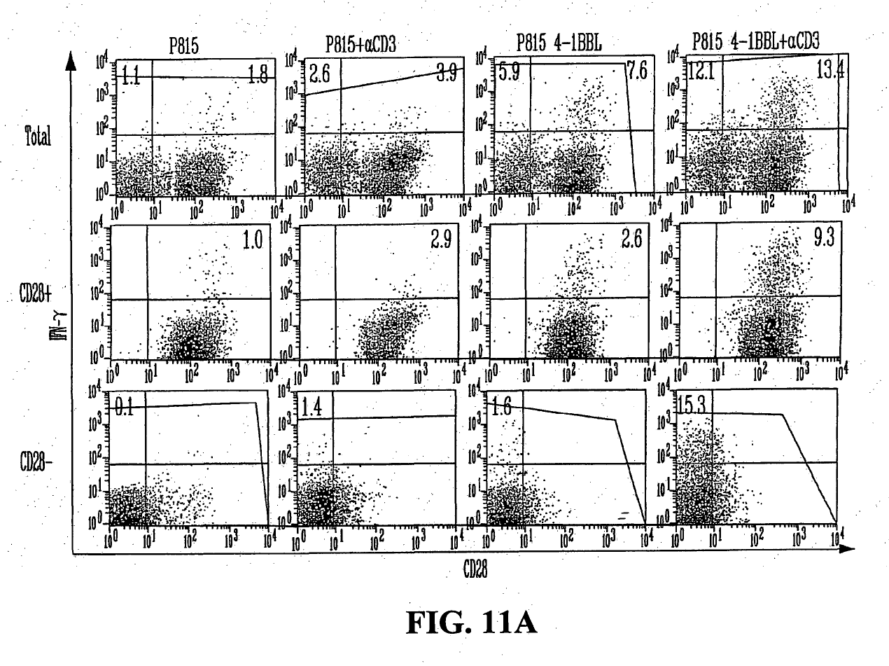

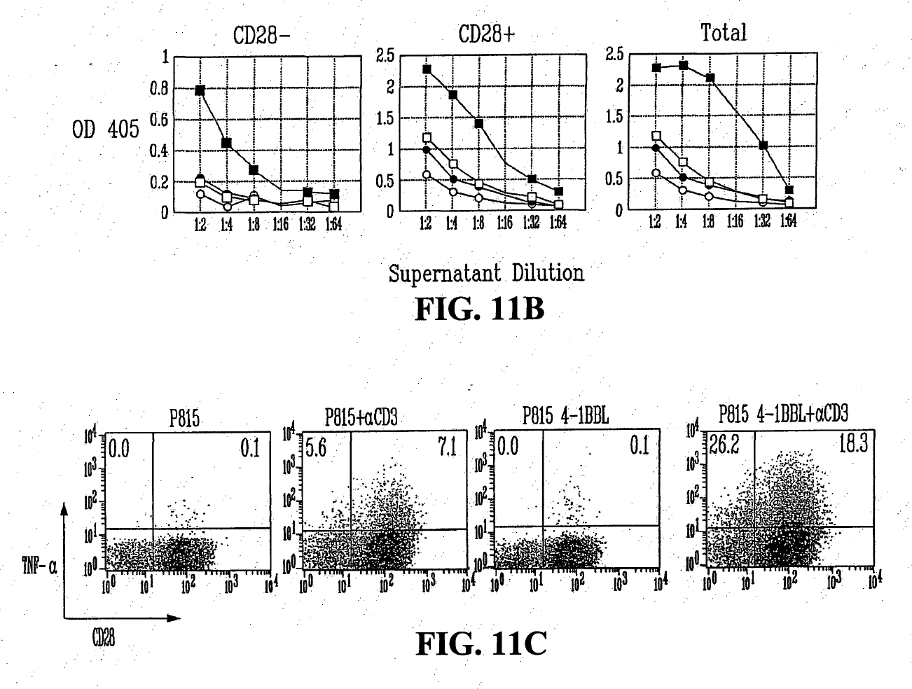

CD28⁺ and CD28⁻ T cells in unfractionated cultures as well as in cultures of sorted CD28⁺ or CD28⁻ T cells produced IFN-γ in response to anti-CD3 plus 4-1BBL stimulation (as measured by intracellular cytokine staining [FIG. 11a] or by ELISA [FIG. 11b]). In donor C, both the CD4⁺ and CD8⁺ CD28⁻ subsets produced IFN-γ (data not shown). Separation of CD4⁺ and CD8⁺ CD28⁻ T cells in this donor and their subsequent stimulation produced similar results (data not shown). CD28⁻ T cells also produced TNF-α in response to anti-CD3 plus 4-1BBL-mediated costimulation (FIG. 11c), although the population was not well resolved and levels of TNF-α are clearly lower than those produced by CD28⁺ T cells.

CD28⁻ T cells failed to produce the Th2 cytokine IL-4, or the regulatory cytokines IL-10 and TGF-β following stimulation with anti-CD3 plus 4-1BBL (data not shown). Unfractionated T cells as well as CD28⁺ T cell cultures produced very small amounts of IL-4 in three out of five donors, whereas IL-10 was produced in barely detectable amounts in only one donor in response to 4-1BBL costimulation. None of the donors showed detectable production of TGF-β (data not shown). Thus both CD4 and CD8, CD28⁺ and CD28⁻ T cell subsets produce predominantly Thl cytokines in response to anti-CD3 plus 4-1BBL-mediated costimulation.

### Example 12

### 4-1BBL Augments the Cytotoxic Capabilities of CD28⁻ T Cells

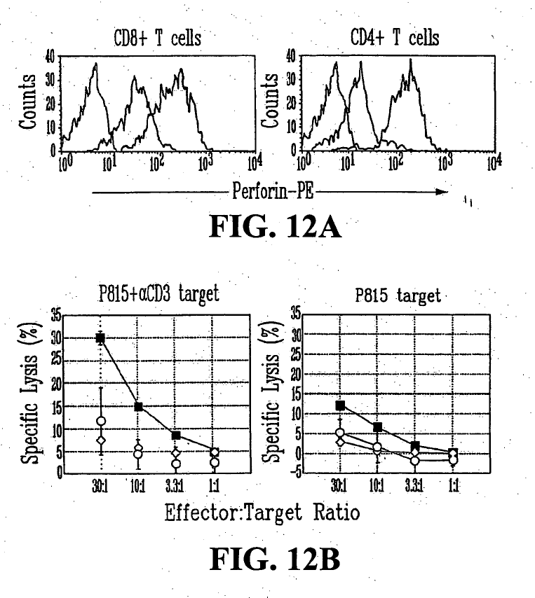

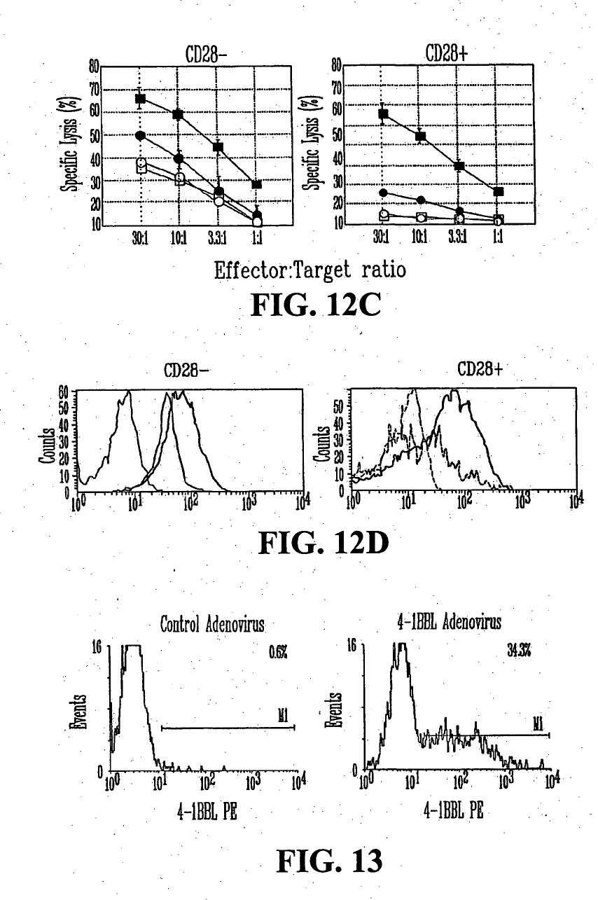

Freshly isolated CD28⁻ T cells were shown to have much higher levels of perforin than CD28⁺ T cells (FIG. 12a). For most donors, these CD28⁻ perforin^{hi} cells are largely of the CD8 phenotype; however, donor C showed high perforin levels in the CD4 and CD8 CD28⁻ population. Consistent with their high perforin levels, and as previously observed (43) freshly isolated unstimulated CD28⁻ T cells were effective in cytolytic function as demonstrated in a redirected lysis assay, in which anti-CD3 is used to direct the CTL killing (FIG. 12b). In contrast, the CD28⁻ cells have lower levels of perforin and show little cytotoxicity above background in the redirected lysis assay (FIG. 12b). These results imply that the CD28⁻ T cells have the constitutive effector function associated with memory cells, although they are low in CD25 and HLA-DR expression, consistent with a resting state. Stimulation with anti-CD3 and 4-1BBL led to enhanced cytotoxic activity in the CD28⁻ T cell subset and a gain of cytolytic effector function in the CD28⁺ subset (FIG. 12c) Following stimulation with anti-CD3 plus 4-1BBL both the CD28⁻ and CD28⁺ T cells showed increased levels of perforin, consistent with their increased cytolytic capacity (FIG. 12d). Neither anti-CD3 alone or 4-1BBL alone resulted in increased perforin levels (data not shown).

### Discussion

### Examples 7-12

The T cell surface protein CD28 provides a critical costimulatory signal for T cell activation. With age, humans accumulate increasing numbers of CD28⁻ T cells and this loss of CD28 expression is exacerbated in certain disease states such as HIV infection, autoimmune conditions or cancer. It is unclear whether CD28⁻ T cells represent terminally differentiated effector cells or whether they remain sensitive to costimulation by CD28-independent pathways. Here, it is formally demonstrated that 4-1BB ligand (4-1BBL) can activate human CD28⁻ T cells, resulting in cell division, cytokine production, enhancement of cytolytic effector function, as well as the upregulation of the anti-apoptotic protein Bcl-X_{L}. The enhancement of effector function and survival of CD28⁻ T cells by 4-1BBL makes it an attractive candidate for a therapy of a disease or condition in which CD28⁻ T cells expansion is observed. One non-limiting example of such a therapy is antiviral therapy such as HIV therapy, where the tremendous expansion of CD8⁺ CD28⁻ T cells results in a large pool of T cells intrinsically incapable of a response to CD28⁻ mediated costimulation.

In the results described herein, it is clearly demonstrated that the 4-1BB/4-1BBL interaction, in the presence of a TCR signal, can costimulate human CD28⁻ T cells to enhance their proliferation, effector function and level of the survival factor Bcl-X_{L}. The immunopotentiation and activation of CD28 T cells is of importance in view of the number of diseases or conditions in which CD28 T cells are expanded. The present invention now provides the means to activate such CD28⁻ T cells (as well as CD28⁺).

An example of a patient in which CD8⁺ CD28⁻ T cells are expanded is HIV patients, where up to 75% of the CD8 T cell pool can be CD28 negative (61). Other conditions also show expanded CD28⁻ T cell populations. For example, in Rheumatoid arthritis, the loss of expression of CD28 on the CD4 T cell pool is associated with the severity of the disease (52, 62). Increased numbers of CD28⁻ T cells are also observed in other autoimmune conditions including systemic lupus erythromatosus and multiple sclerosis (63, 64). Expansion of CD28⁻ T cells is also observed in cancer patients. Of note, and as stated above, CD28-T cells are expanded during aging. The present invention provides the means to activate a significant proportion of T cells in the aging population (53). The progressive loss of CD28 expression could be a mechanism of immuno-senescence or represent a normal function of activated effector cells. Given the findings that CD28⁻ T cells can be induced to proliferate, acquire effector function (cytokine secretion and cytotoxicity), as well as increase the levels of the survival factor Bcl-X_{L} in response to 4-1BB costimulation, these cells are unlikely to represent a purely senescent population of T cells.

Without being bound to a particular theory, the accumulation of CD28⁻ T cells in humans may be due to their mode of prior activation. Support is emerging for the notion that the fate of an effector with respect to CD28 expression lies in the dose of antigen with which the effector cell was activated. Using EBV peptide/MHC tetramers, Hislop et al. (2001, J. Immunol. 167:2019) showed that latent epitope specific T-cells (representing a low antigen dose) were consistently CD45RO⁺ and CD28⁺ while the lytic epitope-specific T cells (representing a high antigen dose) were more heterogeneous, with a distinct CD45RA+CD28⁻ T cell population (65). Thus the high load of antigen during HIV infection may be the driving factor in the emergence of these T cells. An additional factor that may contribute to the accumulation of CD28⁻ T cells in HIV infection is the ability of the viral nef protein to induce downregulation of CD28 on the surface of infected T cells (66).

The ability of 4-1BB to costimulate the CD28 null T cell pool can have important implications in diseases or conditions in which activation, induction of proliferation, acquiring of effector function and/or increased survival of CD28 T cells is desired. The present invention therefore finds utility in HIV therapy, cancer therapies (such as multiple myeloma), where this T cell subset is clonally expanded (53). As such, attempts to utilize only the CD28/B7 pathway for costimulation of T cells will ignore the large pool of CD8⁺ CD28⁻ T cells due to their intrinsic inability to respond. The addition of 4-1BBL to such a regimen should further modulate the response and improve the odds of success, as both CD28⁺ and CD28⁻ T cells would be recruited. In addition, the potential of CD28⁻ T cells to respond to 4-2BBL-mediated costimulation by secreting Th1 cytokines must also be considered in autoimmune disease where the increased CD28⁻ T cell population may contribute to immune pathology. In such a disease, a blockage of 4-1BBL costimulation is predicted to decrease the symptoms.

### Example 13

### Generation of Stimulator Cells

P815 cells from American Type Culture Collection (ATCC, Manassas, Va.) were transfected with full-length human 4-1BBL as previously described (58). Control P815 cells and P815-4-1BB transfected cell lines bind equivalent levels of anti-CD3 (58).

### Example 14

### Flow Cytometry

FITC, PE, CyChrome and biotin conjugated antibodies specific for human 4-1BB, CD28, CD3, CD25, Perforin, CD16,] and CD32 were purchased from BD Pharmingen (Mississauga, Canada). FITC, PE and biotin conjugated anti-human antibodies specific for CD45RA, CD45RO, CD28, CD3, CD4 and CD19 were purchased from eBioscience (San Diego, Calif.). Anti-human Bcl-X_{L} antibody (Southern Biotechnology Associates) was purchased from Cedarlane Hornby, ON, Canada). The hybridomas OKT3, OKT4 and OKT8, secreting antibody specific for human CD3, CD4 and CD8, respectively, were obtained from the ATCC. Antibodies were purified using protein-G Sepharose™ (Amersham-Pharmacia Biotech, Piscataway, N.J.) and conjugated with FITC or biotin (Molecular Probes, Eugene, Oreg.). Streptavidin-APC (eBioscience, San Diego, Calif.) was used as a secondary step to detect biotin-conjugated antibodies. Flow cytometry was carried out on a FACSCalibur™ cytometer (BD Biosciences, San Jose, Calif.) and analyzed with CellQuest™ software (BD Biosciences).

### Example 15

### T Cell Isolation and Purification

Peripheral blood mononuclear cells (PBMC) were isolated from healthy donors by Ficoll-Paque Plus gradient centrifugation (Amersham-Pharmacia, Oakville, Canada). All donors gave informed consent, as approved by the University of Toronto human subjects review board. The PBMCs were incubated for 1 h at 37° C. to deplete macrophages and monocytes through plastic adherence. For the isolation of unfractionated T cells, total T cell immunodepletion columns (Cedarlane Laboratories, Hornby, Canada) were used according to the manufacturer's instructions. CD28⁺ and CD28⁻ T cells were isolated by fluorescence activated cell sorting (FACS). The cells were stained with anti-human CD28 and a cocktail of antibodies specific for human non-T cell markers: CD16, CD19 and CD32. This resulted in a purity of the CD3 population of about 97% (FIG. 8c). Cells negative for CD16/19/32 expression were separated on the basis of CD28 expression. In some donors, where CD4⁺ CD28⁻ T cells were more abundant, anti-CD4 antibody was added to the cocktail to remove the CD4⁺ population, with no significant change in the results for CD28⁻ cells. To counteract the stress of sorting, sorted cell subsets were allowed to recover by an overnight incubation with autologous adherent cells, which were washed 4-5 times with medium prior to the addition of T cells. This incubation resulted in no significant change in the purity of the cultures as determined by flow cytometry analysis pre-and post-incubation.

### Example 16

### T Cell Stimulation

The purified T cells were cultured with irradiated (8,000 rad) P815 cells with or without 4-1BB ligand, coated or not coated with the anti-CD3 antibody OKT3, prepared as previously described (58). Cells were cultured in RPMI 1640 medium (Sigma-Aldrich, St. Louis, Mo.) supplemented as described (58). The ratio of T cells to P815 stimulators was 2:1. Stimulations were performed for 2-6 days, depending on the effector function examined.

### Example 17

### Labelling With CFSE

Cells were washed 2 times with phosphate-buffered saline (PBS), resuspended in PBS containing 2 mM CFSE, and incubated at 37° C. for 20 minutes at a concentration of 5×10⁷ cells/ml. Cells were then washed once with medium, incubated at 37° C. for 15 minutes in medium with 10% FCS and washed once again with medium.

### Example 18

### Cytokines in Culture Supernatants

Matched pairs of anti-human IFN-γ, IL-4, and IL-10 antibodies were purchased from eBioscience. TGF-β ELISA kits was purchased from BD Pharmingen and developed according to manufacturer's instructions. Cultures were stimulated for 5-6 days to measure TGF-β, IL-4, and IL-10 and 2 days for IFN-y and IL-2. IL-2 production was analyzed by measuring [³H] thymidine incorporation of the IL-2-dependent cell line CTLL-2 by pulsing with radioactive thymidine for 7 hours at the end of a 16 hr incubation with culture supernatants. Radioactivity was measured using the Top CountTh scintillation counter (Canberra-Packard, Meriden, Conn.).

### Example 19

### Intracellular Cytokine Analysis

APC conjugated anti-human IFN-γ and TNF-α antibodies and the intracellular staining kit containing GolgiStop™ were purchased from BD Pharmingen. T cells were stimulated for 2 days, incubated for 6h with GolgiStop to inhibit cytokine release, then permeabilized and fixed according to manufacturer's instructions.

### Example 20

### Cytotoxic T Cell Assays

Cytotoxic effector function was measured by a standard ⁵¹Cr release assay. To measure the inherent cytotoxicity, sorted CD28⁻ or CD28⁺ subsets, or purified unfractionated T cells were directly tested for killing of P815 or P815+ anti-CD3 ⁵¹Cr labelled targets. To test the development of cytotoxicity in response to 4-1BBL-mediated costimulation, sorted T cell subsets were incubated in the P815 system for 5-6 days in each stimulator condition. Following the 5-6 day stimulation, cells were collected, live cells were counted on the basis of Trypan Blue exclusion, and incubated with P815 cells plus 0.25 mg OKT3/ml for 4 hours. Radioactivity in the supernatant was determined using the Top Count™ scintillation counter.

### Example 21

### Role of Virally Delivered 4-1BBL in Augmenting Anti-Viral Immunity

This invention also relates to the fact that 4-1BBL delivered to human blood adherent cells using an adenovirus vector can augment anti-viral immunity. Indeed, it is shown that following 4-1BBL delivery to human blood adherent cells, Interferon gamma production increasing in response to a challenge with peptides derived from EBV or influenza was observed.

This provides thus an important additional example, since it shows antigen specificity, anti-viral response as well as an additional mode of delivery that would be suitable for in vivo as well as ex vivo therapy.

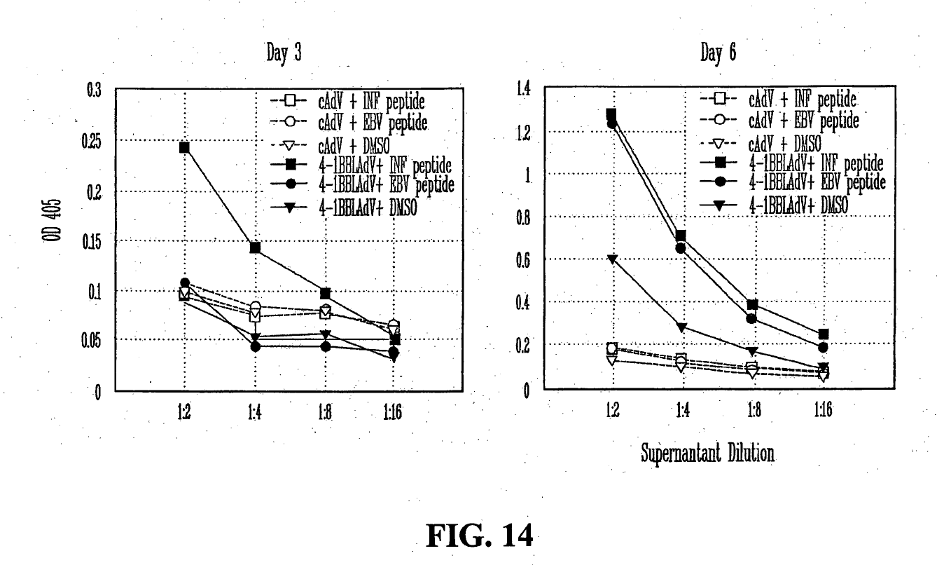

4-1BBL was delivered to antigen presenting cells in a replication defective viral vector (adenovirus V) and used to augment anti-viral immunity. In FIG. 13, it is demonstrated that a recombinant replication defective adenovirus 5 vector expressing the full length human 4-1BBL gene can be used to express 4-1BBL in blood cells (adherent antigen presenting cells) of healthy donors. In FIG. 14, it is shown that the 4-1BBL augments the virus specific response of human blood lymphocytes to peptides from EBV or influenza virus, as measured by an increase in IFN-gamma production. As shown in previous examples, there is good correlation between increased IFN-gamma production and increased cytotoxic T cell function in response to 4-1BBL stimulation. Thus this example shows that 4-1BBL can augment specific anti-viral immunity and that the 4-1BBL can be delivered to human antigen presenting cells using viral vectors, strongly suggesting that this would be applicable to in vivo or ex vivo therapy.

### Example 22

### Materials and Methods for Examples 23-33

Tetramers and Antibodies and Peptides: Class I-peptide tetramers were produced as described in (97). HLA-A2, HLA-B8, and β2-microglobulin (β2m) were cloned in prokaryotic expression vectors and expressed separately in Escherichia coli stains. The heavy chain, β2m and the appropriate peptides were refolded by dilution to yield soluble monomeric class I HLA complexes. The peptides used for HLA-A2 were Influenza-M1 (GILGFVFTL [SEQ ID NO: 7]), EBV-BMLF1 (GLCTLVAML [SEQ ID NO: 8]) and the HIV gag77-85 (SLYNTVATL [SEQ ID NO: 9]). For HLA-B8, the HIV nef 88-95 peptide (FLKEKGGL [SEQ ID NO: 10]) was used. Class I HLA-peptide monomers were biotinylated with BirA enzyme (Avidity, Denver, Colo.) and purified by gel filtration on an FPLC (Amersham Pharmacia biotech). Biotinylated monomers were then mixed with Extravidin-PE (Sigma-Aldrich) at a 4:1 molar ratio (monomer:Extravidin-PE) to form the tetramers.

Antibodies for CD3, CD4, CD28, HLA-A2, and HLA-DR were purified and labeled with FITC or biotin. Antibodies for CD8, CD14, CD27, CD28, CD45RA, B7.1, B7.2, 4-1BBL IFNγ, TNFα, IL-2, Perforin, were purchased from Pharmingen and eBioscience. Bcl-x_{L}-specific antibody was purchased from Southern Biotechnology.

Generation of recombinant adenoviruses: Replication-defective adenovirus 5 expressing human 4-1BB ligand (4-1BBL-Adv) was generated by a two-plasmid rescue method (98). In brief, full length human 4-1BBL CDNA was isolated by RT-PCR method using total RNA extracted from THP-1 cells as (58) and cloned into EcoRI site of shuttle plasmid pDC104. The sequence fidelity of 4-1BBL in the plasmid was confirmed by sequencing analysis. The purified shuttle plasmid was combined with rescue plasmid pBGH1ox-delE1/E3.cre2, and cotransfected into 293 cells to rescue the adenovirus (98). The 4-1BBL transgene expression was confirmed by FACS analysis of 4-1BBL-Adv infected A549 cells. Large scale virus purification was done using virus-infected 293N3S cells. Cells were lysed and the 4-1BBL-Adv or control Adv (transgene-free) viruses purified by cesium chloride gradient ultracentrifugation. The virus titer was measured by plaque assay. A similar protocol was used for the generation of B7.1-Adv (CD80), LIGHT-Adv and OX40L-Adv.

Donors, T cell purification, and APC preparation. 80-120 ml venous blood was obtained from healthy volunteers and PBMC (peripheral blood mononuclear cells) were isolated by Ficoll-Paque Plus gradient centrifugation (Amersham-Pharmacia, Oakville, Canada). All donors gave informed consent, as approved by the University of Toronto human subjects review board. PBMC were used fresh or following freezing in 10% DMSO in 50/50 FCS/Media mixture and storage in liquid nitrogen at −150° C. No significant differences between fresh and frozen samples were observed. Donors were screened for HLA-A2 by staining with the BB7.2 antibody. Donors were confirmed to be HLA-A*0201+ using sequence based HLA-typing (provided by the Canadian Vaccines and Immunotherapeutics Network, CANVAC, MHC typing facility, Toronto Canada).

In most donors studied, a tetramer positive population was detectable in unstimulated PBMC, ranging from 0.05-0.3% of CD8⁺ T cells. To confirm reactivity to influenza and EBV peptides, the responsiveness of each donor was tested by incubating PBMCs with 5 μM of each peptide for 7 days after which Ag-specific T cell expansion was confirmed by tetramer staining.

For costimulation cultures, fresh or freshly thawed PBMC were plated at about 3.5 million/48 well for one hour to allow the adherent fraction to attach to the plastic wells. The non-adherent fraction was removed and kept overnight in a 37° C. incubator. After a washing step, control Adv, 4-1BBL-Adv or B7.1-Adv were added at a multiplicity of infection of 50. To increase the efficiency of viral infection, the plate was centrifuged at 37° C. at 3000 rpm for 1 hour. After the centrifugation step, Influenza Matrix Protein peptide (GILGFVFTL [SEQ ID NO: 7]) or the EBV-BMLF1 peptide (GLCTLVAML [SEQ ID NO: 8]) dissolved in DMSO and diluted in medium were added at the concentrations indicated in the figure legends. Irrelevant melanoma gp100 peptide (IMDQVPFSV [SEQ ID NO: 11]) or DMSO in suspension medium were used as negative controls (indicated on the figures as no antigen), with identical results. After overnight incubation with adenovirus and peptides, APC were washed twice with pre-warmed media. T cells were purified with a Pan T cell negative selection kit from Miltenyi Biotec (MACS) as described by the manufacturer. T cell purity was routinely better than 99.5%, as determined by flow cytometry. Purified T cells were added to adherent cell cultures at a concentration of 1 Million per well of a 48 well dish. The cultures were stimulated for 7-9 days, with the majority of experiments done for 7 or 8 days. In initial experiments, we compared cultures with and without exogenous IL-2 (added at 3 Units/ml) and found that IL-2 did not improve T cell expansion and resulted in increased background, so remaining experiments were done without any exogenous cytokines.

Blood from HIV infected individuals was obtained by CANVAC investigators at the University of Montreal, and HLA type and peptide reactivity determined by CANVAC scientist Nicole Bernard, at McGill University. The acquisition of blood from HIV-infected individuals and samples from University of Montreal and distributing samples to collaborators and the University of Toronto was approved by the ethics board of University of Toronto. Blood samples were processed as above for healthy donors except that stimulations were done with HIV peptides. The density of PMBCs that were plated was increased to 4-5 million per 48-well in order to obtain enough adherent monocytes.

Flow cytometry: Samples were stained with tetramers at 37° C. for 15 minutes, followed by a wash with cold buffer. All subsequent antibody stains were done on ice. For intracellular cytokine staining, samples were restimulated with 5 μM peptide for 6 hours in the presence of GolgiPlug™ (BD-Biosciences). Cells were stained for surface markers, followed by intracellular staining with anti-cytokine antibodies. The CytoFix/CytoPerm kit (BD Biosciences) was used to fix and permeabilize the cells for intracellular staining. FlowJo software was used for data analysis.

CTL assays: The human T2 cell line was used for target cells for A2-positive individuals. Targets were pulsed with 3 μM Influenza, EBV or HIV peptides and DMSO in suspension medium or irrelevant melanoma control peptides overnight. The following day the cells were labeled with 200 μCi of Na₂⁵¹CrO₄ and incubated with effector T cells for 4 hours. Supernatant was analyzed for the release of radioactive chromium. For HLA-B8+ HIV patients, autologous EBV transformed B-LCL cell lines were used as target cells. The cells were labeled with B8-restricted peptide and Na₂⁵¹CrO₄ for one hour at 37 degrees C.

### Example 23

### Methodology for Conversion of Peripheral Blood Monocytes Into Antigen Presenting Cells.

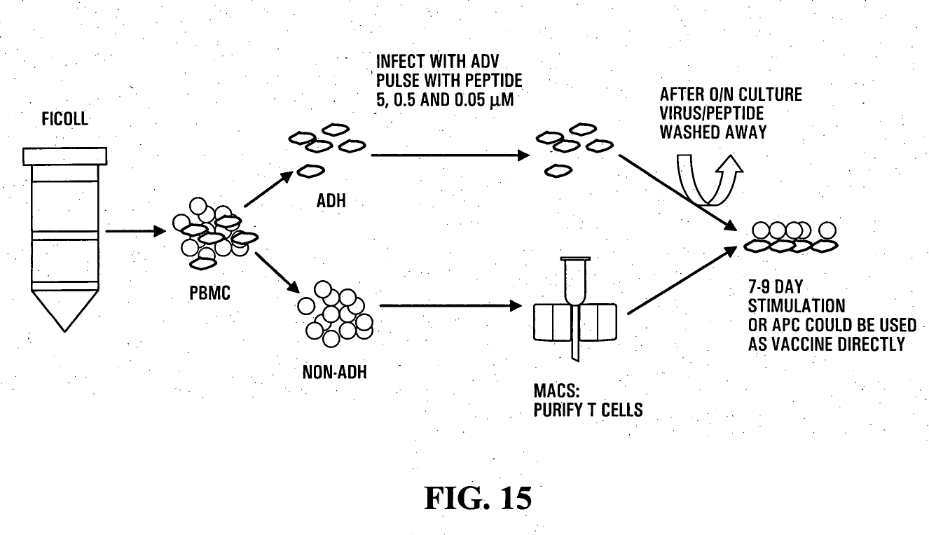

FIG. 15 describes the methodology for converting peripheral blood monocytes from patients or healthy donors into antigen presenting cells using recombinanant adenovirus and peptide antigens. Mononuclear cells are purified from whole blood by Ficoll density gradient centrigugation. Adherent cells are separated from non-adherent cells by a brief incubation on tissue culture plastic as described in the methods. T cells are purified by depleting unwanted subsets as desribed in the methods. Adenovirus and peptides are added to the adherent cells overnight and washed away the next day. The modified adherent cells (APC) can be used in vitro as shown in the subsequent examples, or could be used as immunogens in vivo, as described for dendritic cells.(71).

### Example 24

### Characterization of Adherent Cells and Gene Transfer Efficiency With Recombinant Adenoviruses

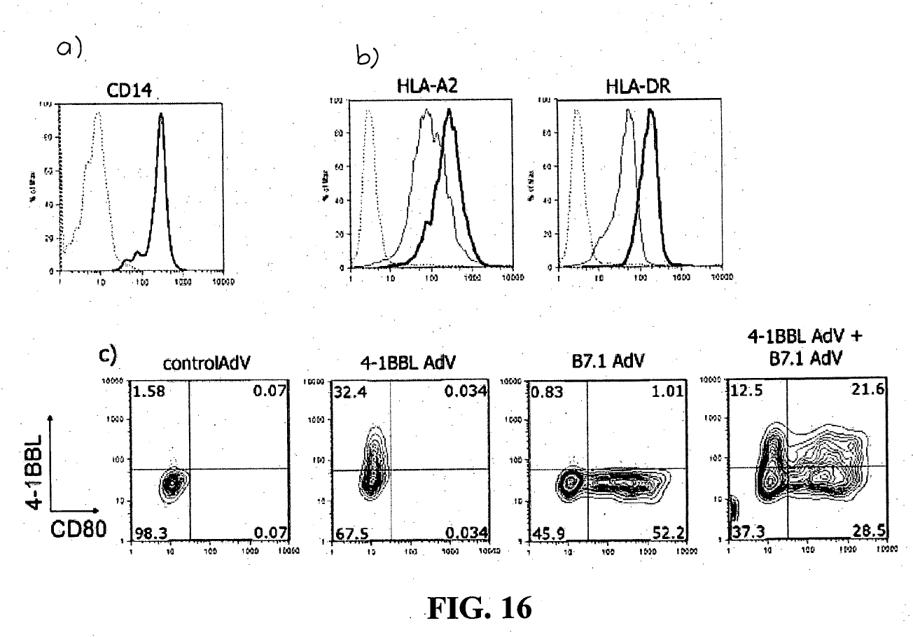

Replication defective recombinant adenoviruses were used to deliver costimulatory molecules to the adherent cell fraction of healthy donor PBMC. The adherent cell fraction was almost exclusively composed of CD14 monocytes (FIG. 16a) with no detectable B7.1 and negligible 4-1BBL (<2%) expressed on freshly isolated cells. Some B7.2 (CD86) expression was detected on monocytes of most donors (FIG. 16b, day 0). These levels significantly decreased after 2 days in culture with control or 4-1BBL-adenovirus (FIG. 16b, day 1-2). Adherent monocytes expressed Class I (HLA-A2) and Class II (HLA-DR), both of which were upregulated after infection with adenoviruses (FIG. 16a). The delivery of both costimulatory molecules through recombinant adenoviruses resulted in 35 to 55% of monocytes expressing 4-1BBL or B7.1 (FIG. 16c). Detailed legend—FIG. 16: Adherent cells from a healthy donor were stained for expression of a) CD14 (monocytes), b) HLA-A2 and HLA-DR before (thin line) and 24hr after (thick line) infection with adenovirus. Similar results were obtained using control-Adv or 4-1BBL-Adv. (not shown). Dashed line indicates staining with isotype control antibody. c) Expression of B7.1 and 4-1BBL 24hrs after infection with control or recombinant adenoviruses, as indicated above each panel. In b and c, percent of cells in each quadrant are indicated. Results are representative of 3 different donors.

### Example 25

### Effect of 4-1BBL on Expansion of T Cells From Healthy Donors. 4-1BBL Acts as a Potent Adjuvant for Influenza- and EBV-Specific Memory CD8 T Cell Responses

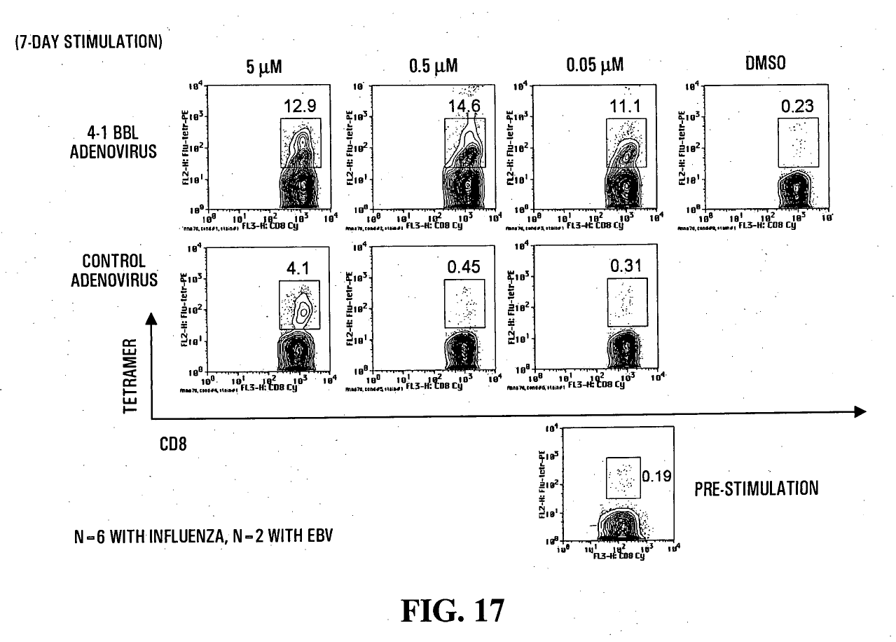

To test the effect of 4-1BBL on memory responses to viruses, T cells from healthy donors were incubated with adherent monocytes (FIGS. 15 and 16) that had been pre-activated with 4-1BBL-Adv or control Adv plus influenza matrix peptides. 4-1BBL costimulation was tested over a 100-fold range of peptide concentrations and significantly enhanced the expansion of influenza-specific tetramer+ T cells over control adenovirus treated cultures at all peptide doses. At the lowest peptide dose, a 35-fold enhancement of T cell expansion by 4-1BBL-Adv over control Adv-treated cultures was observed (FIG. 17). Details of FIG. 17: T cells were cultured with adherent monocytes preincubated with 4-1BBL-AdV or control-AdV and influenza matrix peptide at the concentrations indicated above each panel. Seven days later, cells were analyzed for CD8 and HLA-A*0201/influenza matrix peptide tetramer staining. Prior to stimulation, total purified T cells contained memory CD8 T cells specific for the influenza matrix protein epitope (range 0.05-0.27% of CD8 T cells in 7 donors).

### Example 26

### 4-1BBL-Expanded T Cells are Better Able to Kill Cells Displaying Influenza Peptide

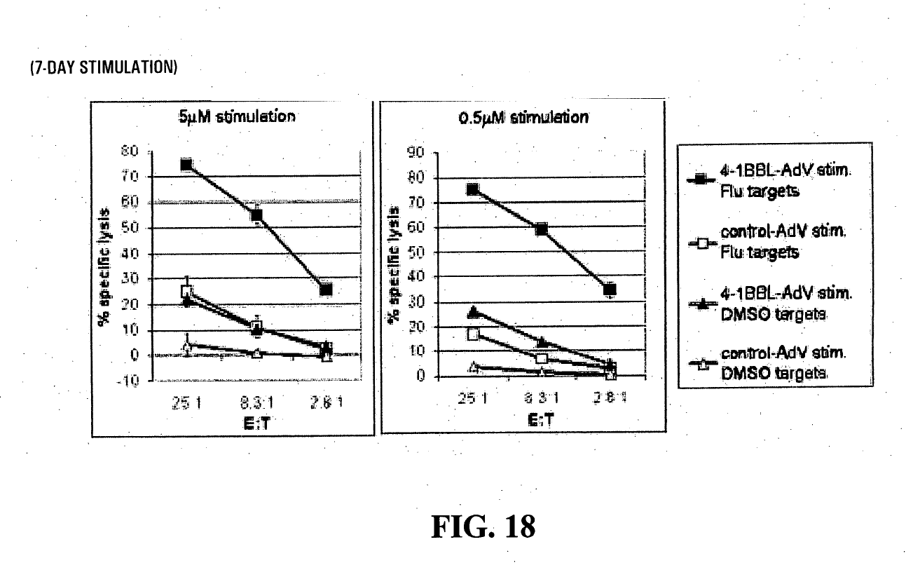

The ability of 4-1BBL stimulated T cells to kill influenza-peptide pulsed targets was consistently better than observed with control adenovirus plus peptide treated cultures, correlating with the increased expansion of tetramer positive cells (FIG. 18) and increases in effector molecule expression. Details of FIG. 18: Killing of influenza peptide coated T2 targets cells by effector T cells from FIG. 18, with 7-day stimulation conditions as indicated in the legend.

### Example 27

### Induction of Cytokine Production by 4-1BBL-Expressing APC

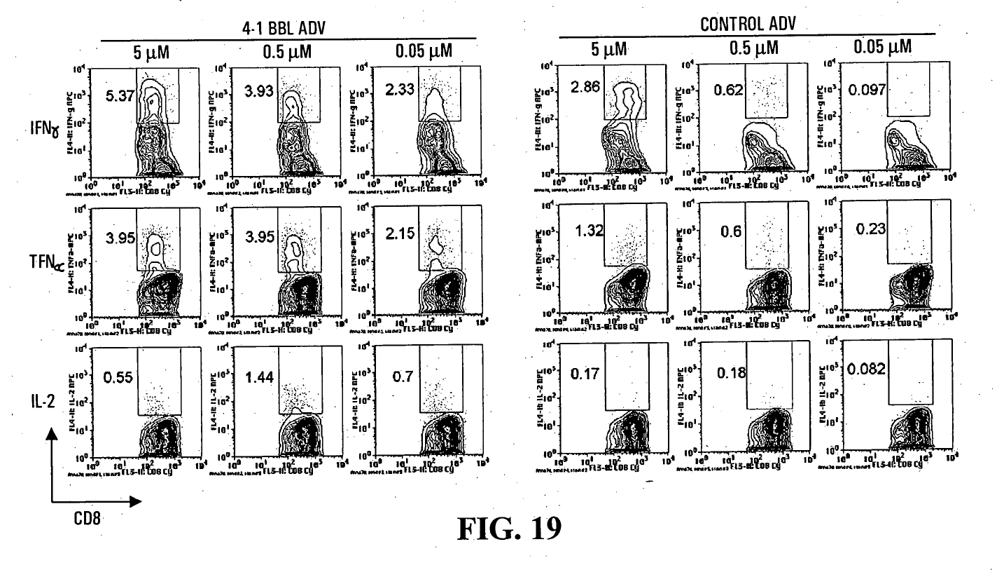

Cultures with 4-1BBL costimulation showed efficient production of cytokines after a 6h restimulation with the peptide (FIG. 19), whereas control adenovirus cultures showed moderate production of cytokines only in cultures that received a high dose of peptide during the 7 day culture period. In some donors, peptide restimulation for cytokine analysis was performed at 0.15 μM. Even this low dose of antigen resulted in nearly similar production of IFNγ in the 4-1BBL costimulated cultures, as compared to restimulation with 5 μM of peptide (data not shown). Control adenovirus cultures showed no significant response at this low restimulation dose, suggesting that 4-1BBL costimulated T cells have an increased sensitivity and respond well at lower concentrations of peptide. The T cells produced mainly IFNγ and TNFα, and relatively small amounts of IL-2: a phenotype associated with memory responses. Since IL-2 was not added to the cultures (see methods) this small amount of IL-2 may be important in the observed CD8 T cell expansion. Details of FIG. 19: Cytokine production by 4-1BBL or control stimulated T cells after 5h restimulation in the presence of monensin and 5 μM Influenza-peptide, measured by intracellular flow cytometry. Concentrations above plots indicate concentration of peptide during stimulation. Percent of cells in the indicated population are included in the plots for a) and c). Similar results were obtained with 6 donors, with one to four experiments per donor. EBV specific T cells showed similar results in 3 donors.

### Example 28

### Comparison of 4-1BBL and B7.1 or the Combination for Stimulation of Influenza Specific CD8 T Cells

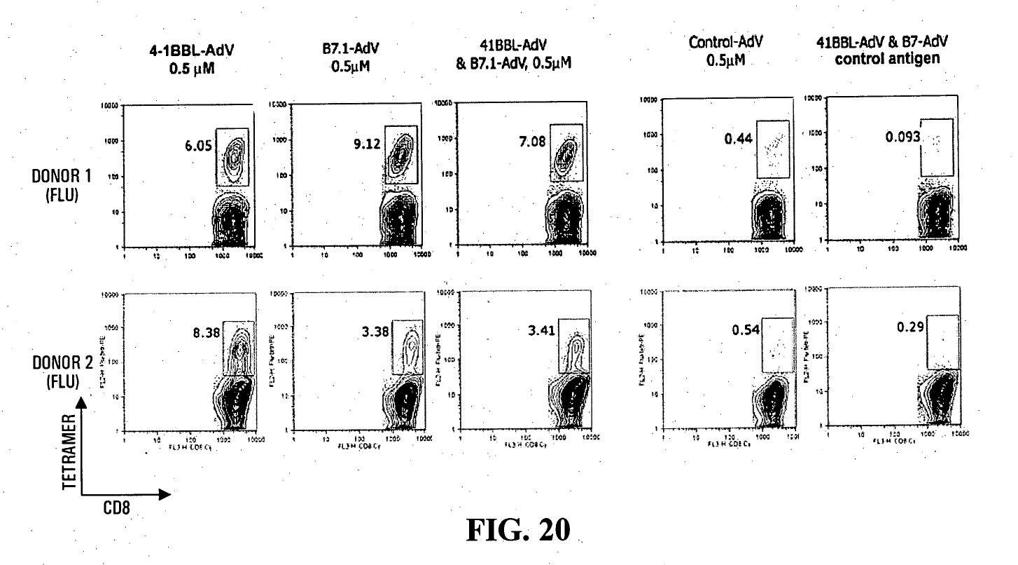

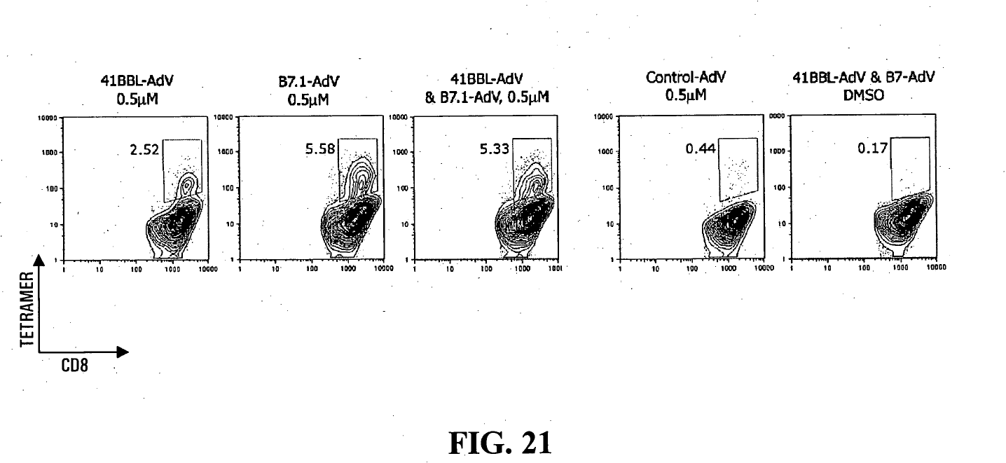

The preceding data suggest that 4-1BBL is an effective costimulatory molecule for memory cytotoxic T cells. To determine the effectiveness of 4-1BBL compared to a more conventional costimulation regimen, we set up parallel cultures in which 4-1BBL, B7.1 or both were delivered to the APC, with the results shown in FIG. 20. In all donors tested, 4-1BBL and B7.1 could each enhance T cell expansion when tested in isolation, albeit with variability in efficacy between donors. In some donors, B7.1 appeared as good or better as 4-1BBL in expanding influenza specific T cells, but further kinetic anaysis revealed that 4-1BBL induced more rapid expansion of the CD8 T cells than B7.1. Thus for healthy donor influenza specific responses, 4-1BBL is better than B7.1 at expanding memory anti-viral CD8 T cells. Surprisingly, the combination of 4-1BBL and B7.1 did not show additive or synergistic effects over individual costimulatory molecules in any of the donors examined, even when analyzed at earlier time points. FIG. 21 shows a similar analysis for EBV-specific responses.

Details of FIGS. 20 and 21: Expansion of tetramer+ T cells in response to Influenza after eight days of stimulation. Plots are gated on CD8⁺ events. Numbers in each plot indicate percent of CD8 T cells staining with tetramer. Representative of 3 different donors for influenza (FIG. 20) and 2 different donors for EBV (FIG. 21). The stimulation conditions used to modify the APC are indicated above the plot. Results are shown as Tetramer staining versus CD8 staining and the numbers indicate % of CD8 T cells specific for the influenza (FIG. 20) or EBV (FIG. 21) MHC-peptide tetramer.

### Example 29

### 4-1BBL is Equal or Better Than B7.1 in Increasing Expression of Survival and Effector Molecules in Virus Specific T Cells From Healthy Donors

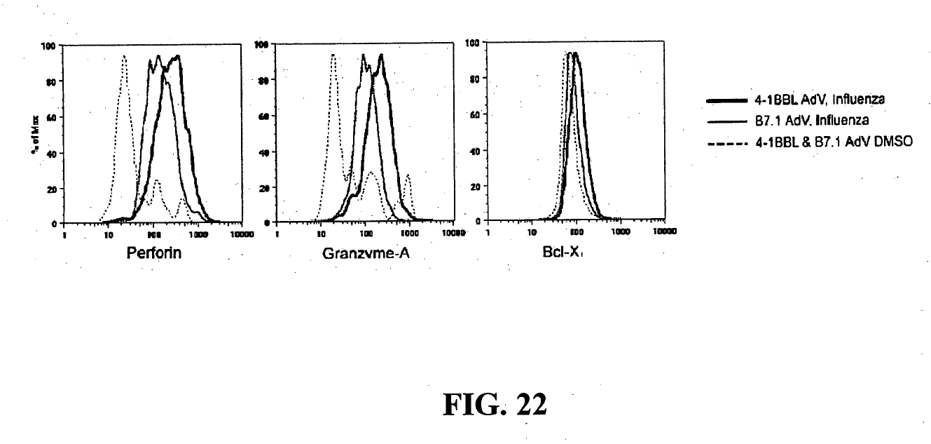

As shown in FIG. 22, T cells were cultured with adherent monocytes preincubated with 4-1BBL-AdV, B7.1-Adv or control-AdV and Influenza matrix protein 1 peptide or EBV BMLF1 peptide at the concentrations indicated above each panel. Eight days later, cells were analyzed for Intracellular levels of Perforin, Granzyme-A and Bcl-x_{L}, as measured by intracellular flow cytometry. 4-1BBL, B7.1 and no-costimulation cultures are compared as indicated. In general we find that perforin levels correlate well with cytotoxic activity (data not shown).

### Example 30

### Effect of B7 and 4-1BBL on Expansion of T Cells From HIV Infected Donors

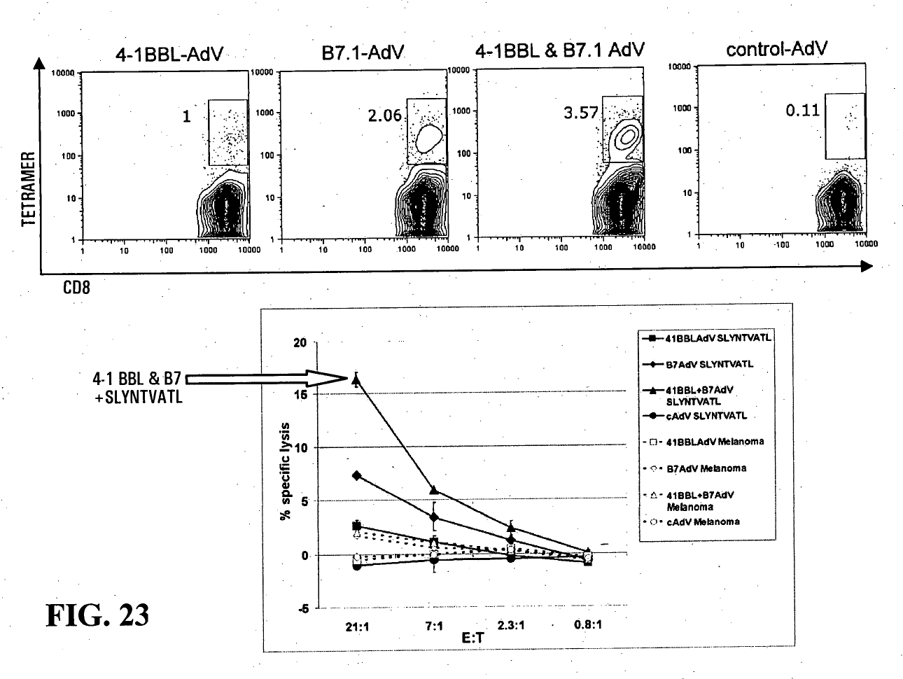

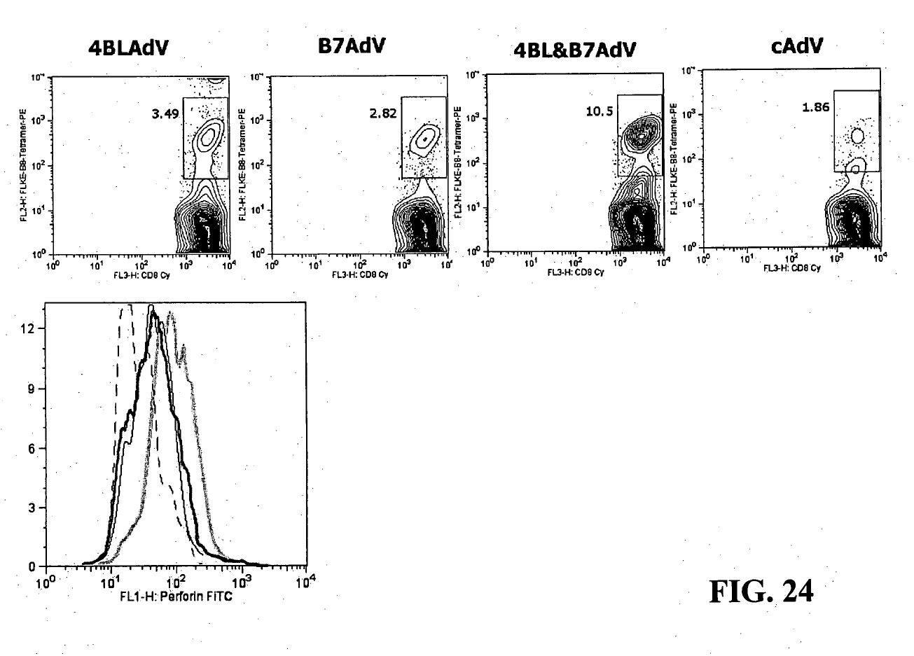

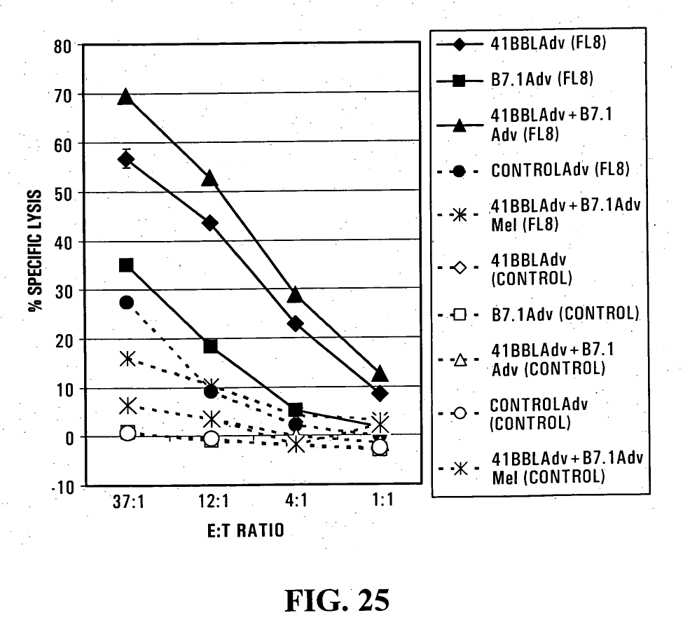

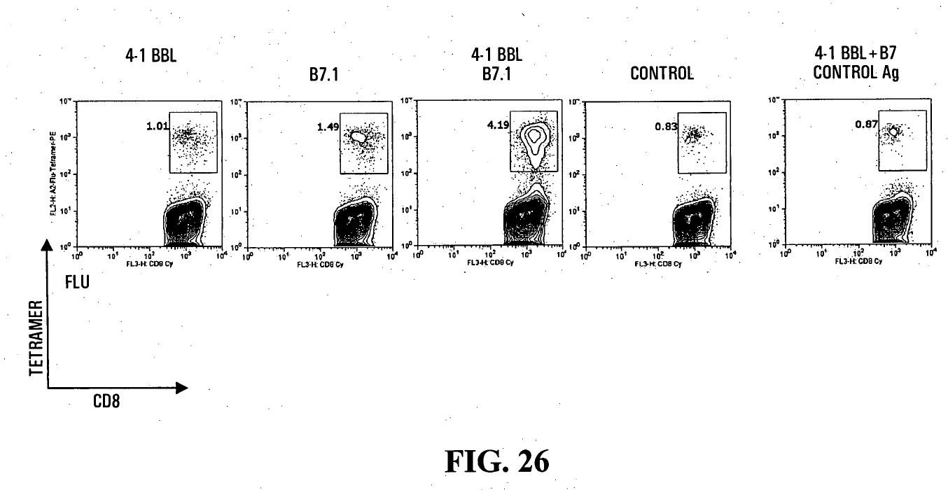

For HIV infected donors, the combination of B7+4-1BBL gives improved expansion of T cells and greatly improved induction of HIV peptide specific cytotoxic T cell activity over either stimulus alone. HIV Patient Responses to 9-day A2.1 HIV gag peptide (SLYNTVATL [SEQ ID NO: 9]) stimulation are shown in FIG. 23. Similar results were obtained with an HLA-B8 donor responding to HIV nef 88-95 (FLKEKGGL [SEQ ID NO: 10]). FIG. 23 shows tetramer versus CD8 staining by flow cytometry following a 9 day culture with APC treated peptide and with control-Adv, 4-1BBL-Adv, B7.1-Adv or both, as indicated above each panel and described in FIG. 15. The lower panel of FIG. 23 shows that T cells stimulated with adherent cells modified with both 4-1BBL and B7.1 give superior killing of HIV gag (SLYNVATL [SEQ ID NO: 9]) coated target cells. In FIG. 24, a similar experiment is shown with an HLA-B8 donor and analysis of cytotoxic effector function by staining for perforin reveals that the combination of 4-1BBL and B7.1 are more efficient in upregulating perforin (grey line). FIG. 25 shows the cytotoxic T cell activity against HIV peptide coated targets of the T cells generated in FIG. 10. In this donor, 4-1BBL alone induced substantial cytotoxic activity but there was some improvement when both molecules were added and B7.1 alone was much weaker. In general we have found a correlation between the number of virus-specific tetramer positive cells and the CTL activity observed. Analysis of an HIV patient for responses to influenza virus also revealed that the combination of B7.1 and 4-1BBL were required for maximal activity (FIG. 26). Thus the difference in HIV patients versus healthy donors in costimulatory requirements seems to be in the state of the immune system rather than the epitope per se.

### Example 31

### Fold-Expansion in Five Healthy Donors to Influenza and EBV Epitopes, Showing Lack of Enhancement With the Combination of 4-1BBL and B7.1 Costimulation

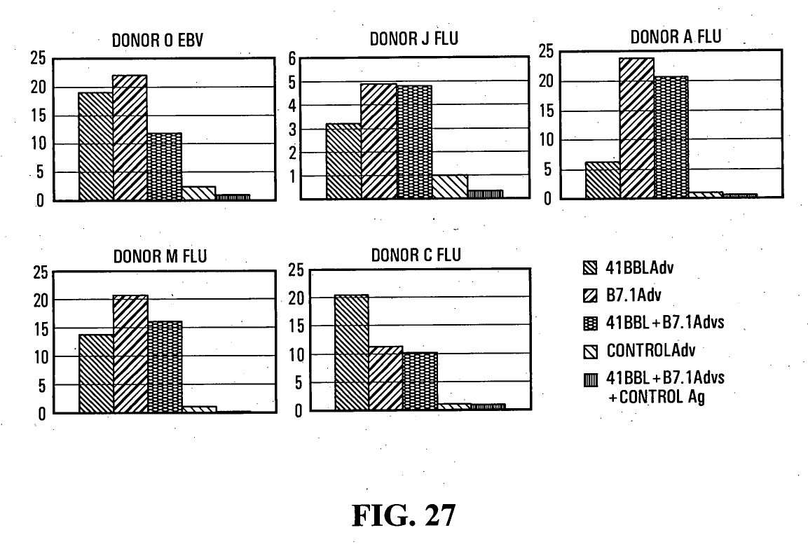

Fold expansion was determined by dividing the expansion obtained in costimulated cultures (4-1BBLAdv, B7.1Adv or combination) by the expansion in control cultures (controlAdv or 4-1BBLAdv+B7.1Adv with irrelevant antigen). Results for a representative donor are shown in FIG. 27.

### Example 32

### Fold-Expansion in Six HIV Patients to HIV or Influenza Epitopes, Showing Additive/Synergistic Effects with the Combination of 4-1BBL and B7.1 Costimulation in Chronically Infected Patients (>5 years).

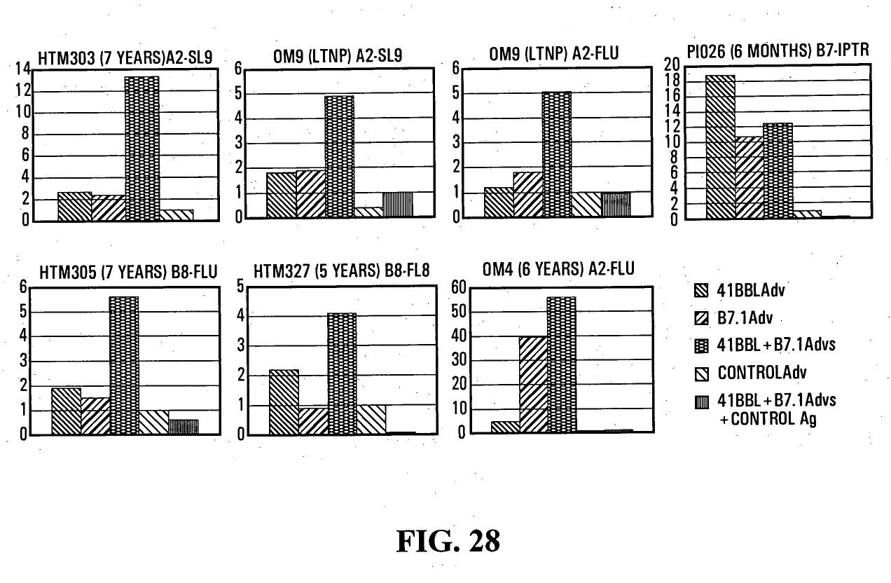

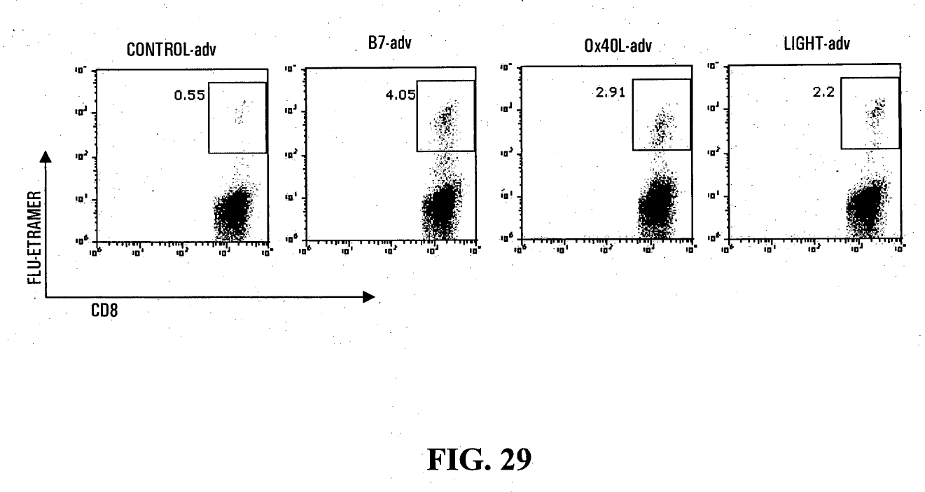

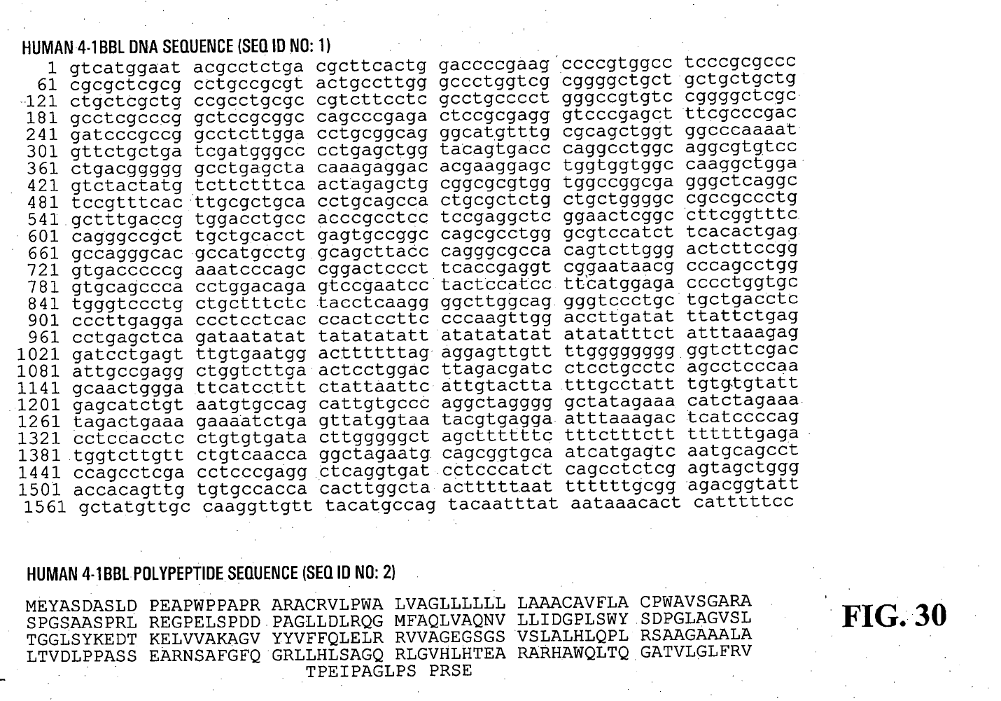

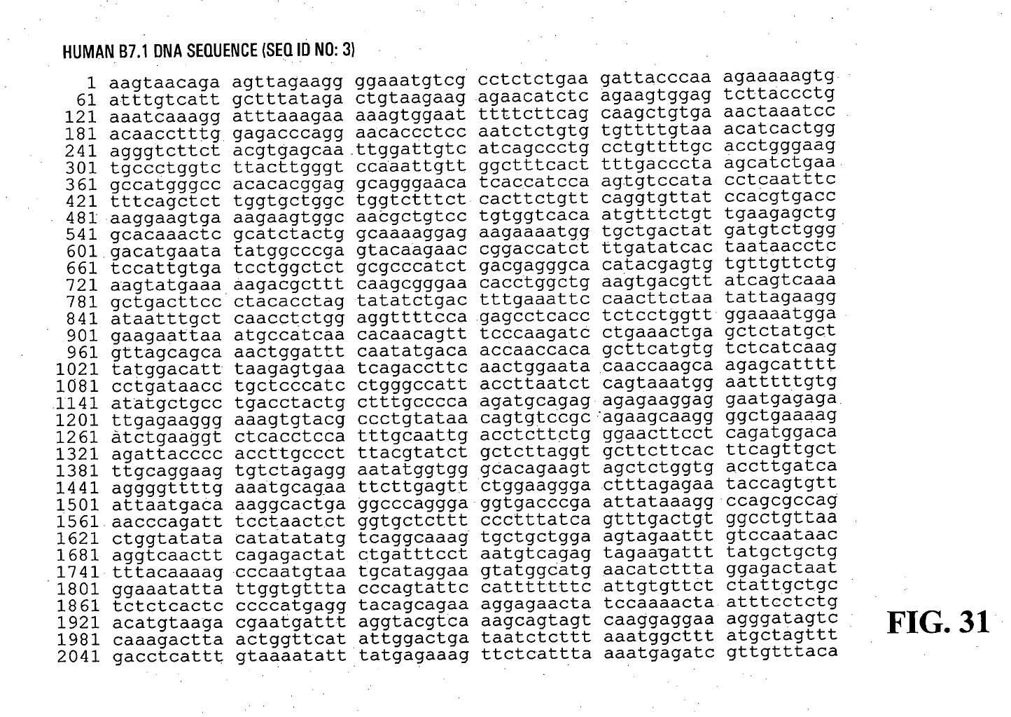

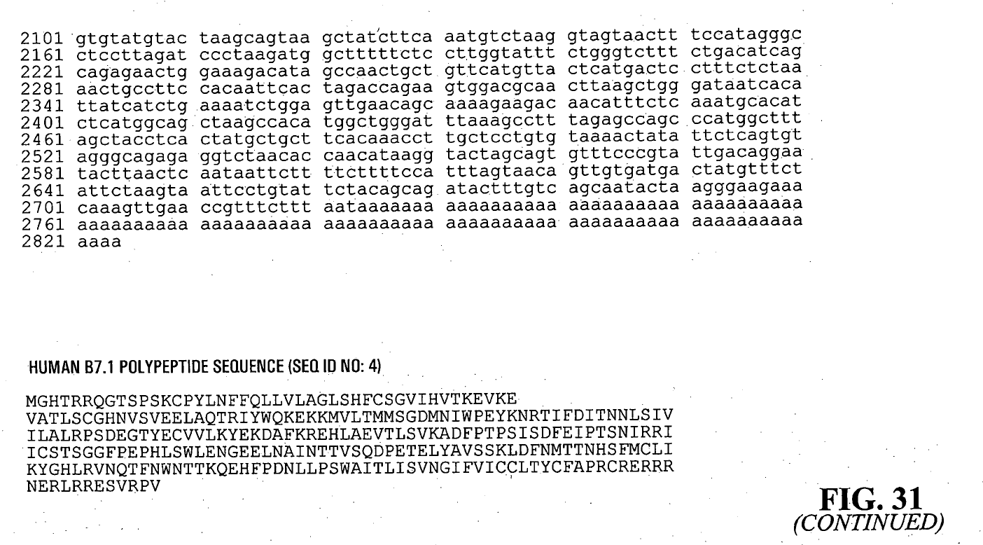

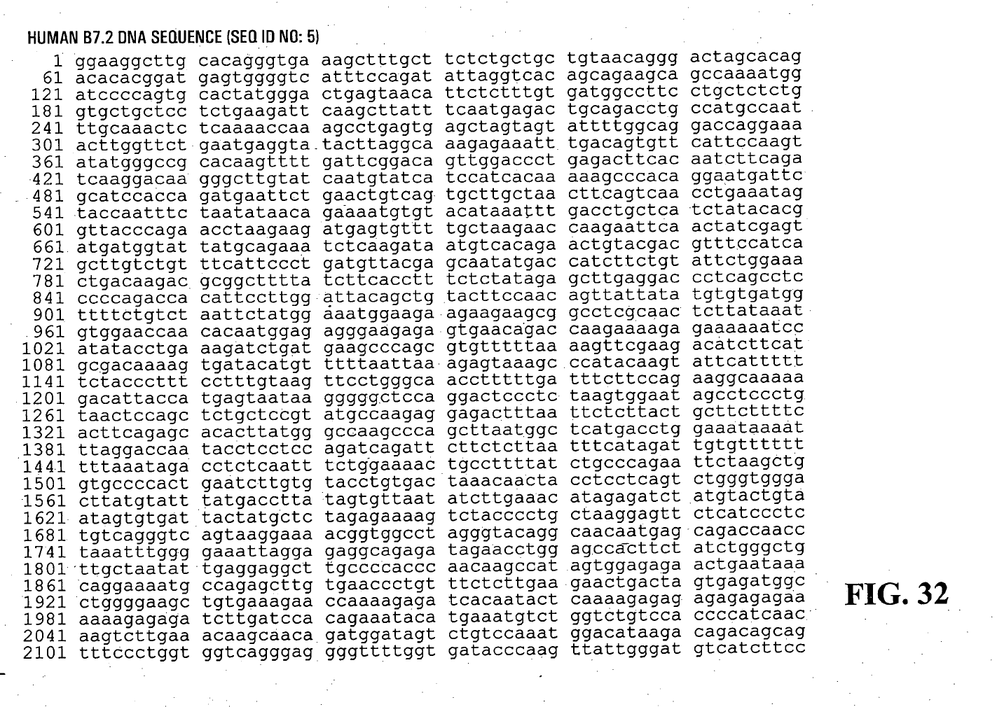

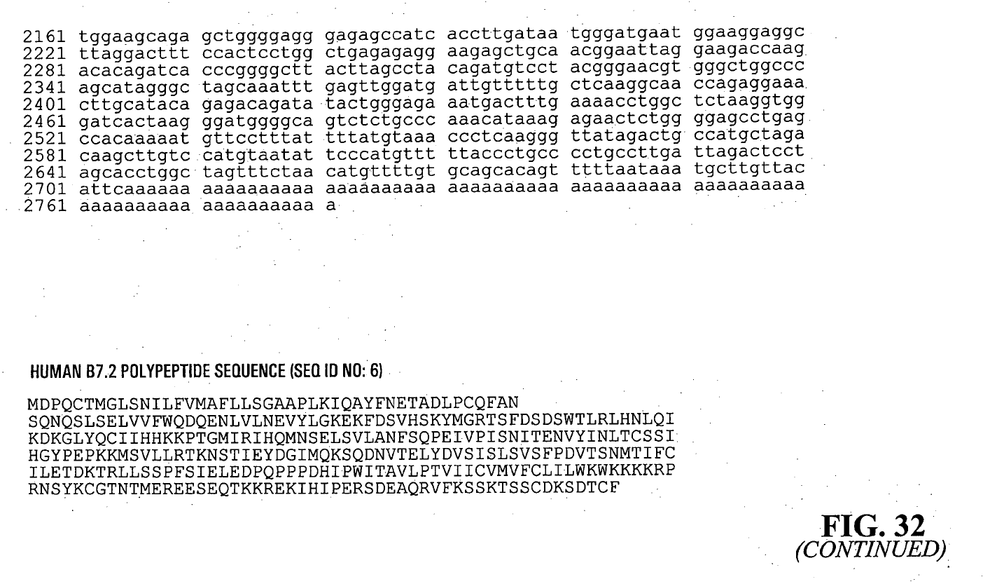

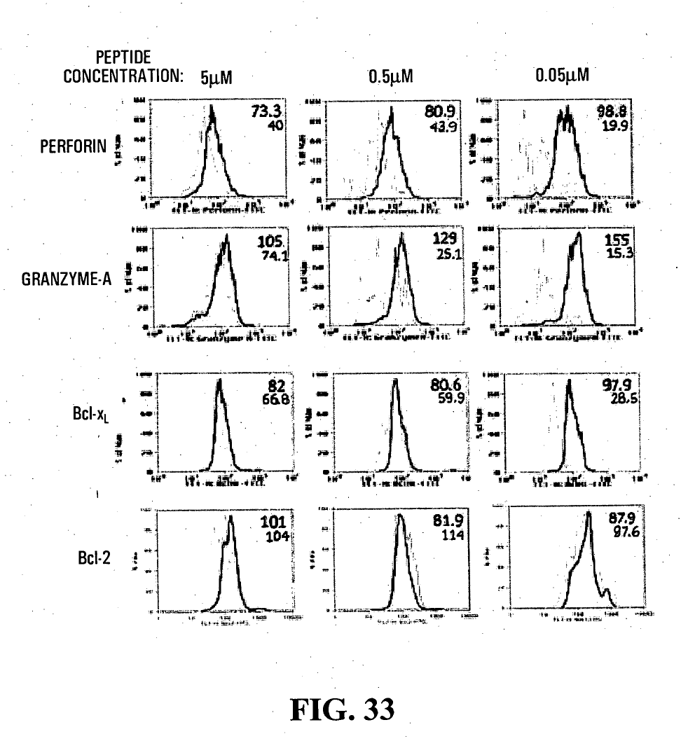

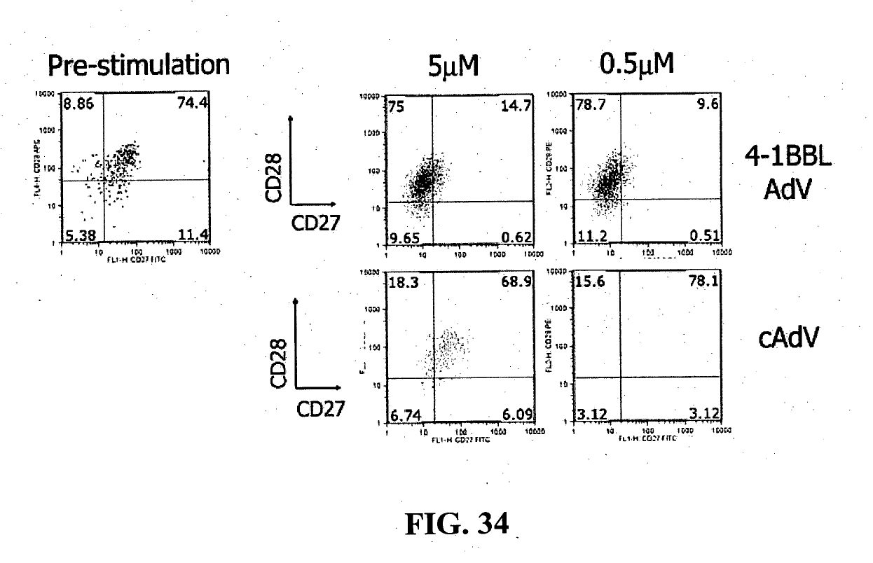

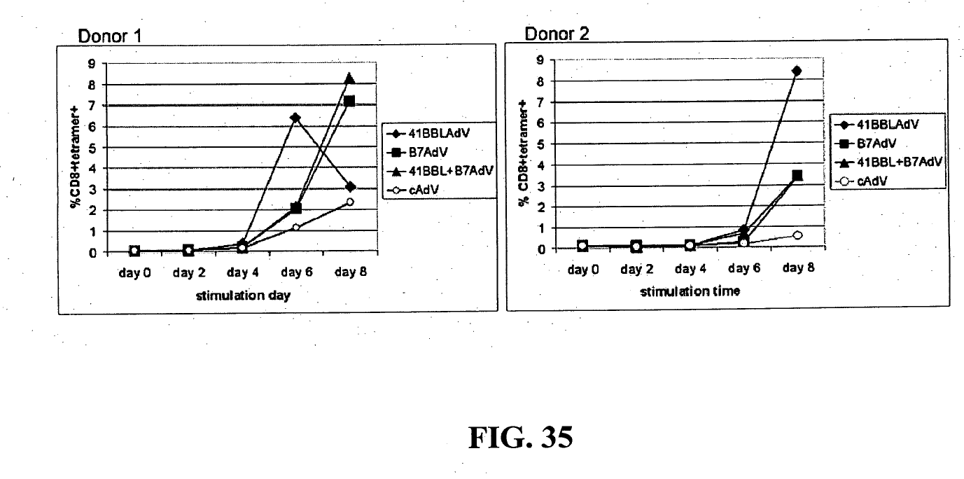

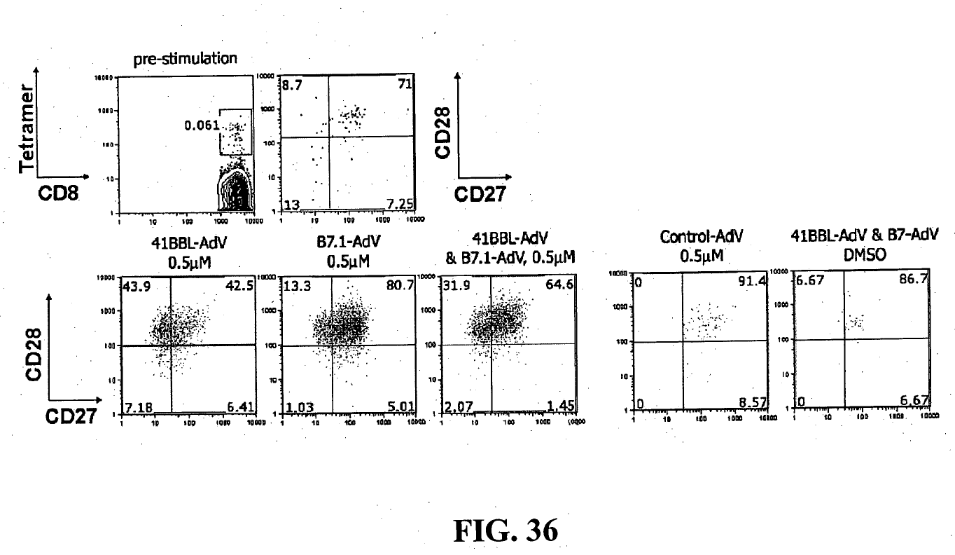

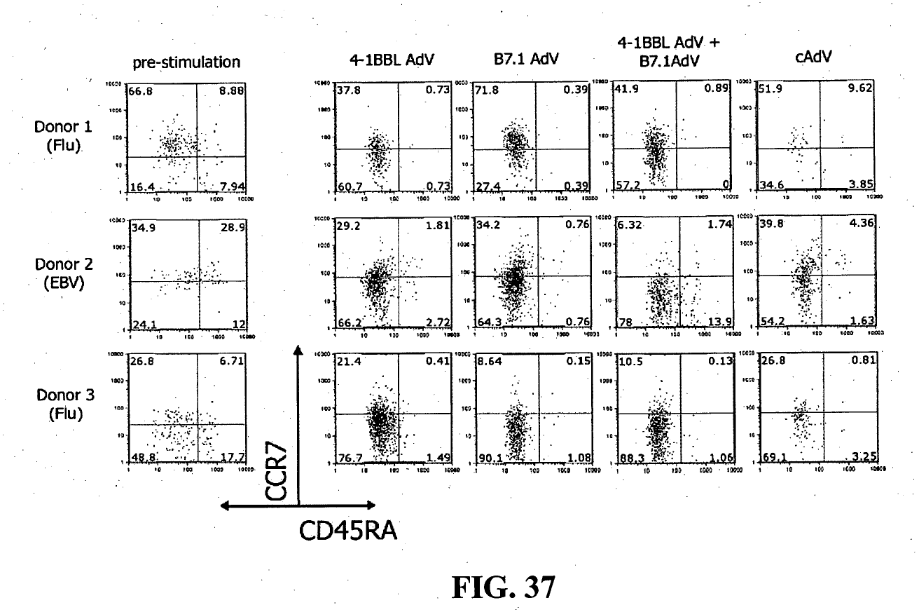

Results are shown in FIG. 28. Length of infection and epitope are indicated. One recently infected patient (˜6 months, preliminary data) did not show this effect in one experiment. Fold expansion was determined by dividing the expansion obtained in costimulated cultures (4-1BBLAdv, B7.1Adv or combination) by the expansion in control cultures (controlAdv or 4-1BBLAdv +B7.1Adv with irrelevant antigen). Tetramer percentages or actual cell numbers were used for this determination.

### Example 33

### Other Members of the TNF Family, Delivered by Adenoviruses to Human Monocytes, Can Also Enhance the Ability of Monocytes to Activate Influenza-Specific T Cells

Several other members of the TNFR family play a role in T cell activation. OX40 (CD134)/OX40L, CD27/CD70, 4-1BB (CD137)/4-1BBL have been fairly well established as having stimulatory activity for T cells activation (reviewed in (75, 83). In addition, the TNFR/TNF ligands CD30/CD30L, HVEM/LIGHT and GITR/GITRL also appear to have stimulatory effects for T cells. (99-102; Gurney et al. 1999, Current Biol. 9:215),. Therefore it is likely that each of these molecules will have activity in augmenting the ability of monocytes to activate T cells.

OX40 is primarily expressed on activated CD4 T cells and for primary responses its expression is dependent on CD28 signaling (103, 104). OX40 signaling in T cells augments CD4 responses at least in part by enhancing T cell survival subsequent to CD28 stimulation (105-108). Thus OX40 is primarily thought of as a costimulatory molecule for CD4 T cells. However, there is some evidence in mice that anti-OX40 can augment CD8 T cell responses (109).

LIGHT is a TNF ligand that can bind to HVEM as well to the LTP receptor. (68) Studies of LIGHT^{-/-} mice in general show a positive role for LIGHT in immune responses (100, 101, 111) In some studies effects were limited to CD8 T cells (100, 111), although another study showed effects on CD4 T cell IL-2 production in an MLR (101). In the absence of CD28-signaling, LIGHT influences both cardiac and skin graft rejection (101, 112), and in this regard, appears similar to 4-1BBL (14). However, in contrast to the effects of 4-1BBL on secondary CTL responses (14) there was no defect in ex vivo CTL responses or antibody responses following VSV infection (101).

We therefore show herein that the TNF family ligands LIGHT and OX40L can also induce in monocytes the ability to allow expansion of influenza specific CD8 memory T cells. These data suggest that one can use either 4-1BBL, LIGHT or OX40L for augmenting the T cell activation ability of monocytes and by extrapolation to the studies with HIV patients, a combination of B7.1 and one or more of the TNF ligand family will give best results. To our knowledge this is the first evidence that LIGHT and OX40L can directly stimulate human CD8 memory T cells. In addition, to 4-1BBL, LIGHT and OX40L, the closely related TNF family members CD30L, CD70 and GITR-L are likely to have similar effects.

Therefore, for 7 healthy donors, 4-1BBL modified monocytes are sufficient for augmentation of anti-viral cytotoxic CD8 T cell memory cell expansion and development of effector function, with no added benefit of adding B7.1 to the monocytes (analyzed for 5 of the donors—FIG. 26). In contrast, for 5 out of 6 HIV-infected donors, we find that the memory anti-viral CTL responses (tested on either HIV gag, HIV nef or influenza epitopes) are improved by combining 4-1BBL and B7.1 adenovirus modification of adherent cells (FIG. 27). All five chronically infected patients (>5 years), including one long term non-progressor (LTNP) patient, showed added benefit of both 4-1BBL and B7.1 costimulatory molecules. The only HIV patient not exhibiting this effect was a patient infected relatively recently (˜6 months). While it is possible that only chronically infected patients benefit from both costimulatory molecules, this results needs to be repeated in this donor and other recently infected donors. Further work is being carried out to analyze a greater number of donors to confirm these differences between healthy donors and HIV infected individuals.

Since most HIV-specific CD8 T cells in the donors analyzed are CD27⁺ CD28⁻ (65-90%) and most Influenza and EBV-specific CD8 T cells in healthy donors are CD27⁺ CD28⁺, the differences in responses might be due to the low CD28 expressed on HIV T cells. This is not the case, however as Influenza-specific CD8 T cells in HIV patients, despite a mainly CD27⁺ CD28⁺ phenotype (90-100%), do in fact show an additive effect with 4-1BBL and B7.1 costimulation (FIG. 27). The levels of tetramer+ T cells are rather high in HIV donors examined, ranging from 0.8-1.6%, yet up to 75% of these T cells die during the first day of culture. This contrasts with healthy donors, where most of the tetramer+ CD8 T cells remain alive. As a result, the HIV patients in FIG. 27 that show a rather high fold expansion in fact show lower than expected numbers of tetramer+ CD8 T cells: the high expansion is due to extensive cell death in the control cultures and a subsequent lack of expansion. This might be useful in adoptive transfer therapy, as only the best fit effectors will survive the culture conditions and most anergic or non-reactive effectors will be eliminated. The results suggest that for immunotherapy of HIV patients it will be important to include B7.1 and 4-1BBL or antibodies to their receptors in order to maximize cytotoxic T cell activation. Since cancer patients also show a weakened immune response, the prediction is that other patients with chronic infectious or neoplatic conditions will show a similar requirement. Other members of the TNF family including OX40L, LIGHT and possible CD30L, CD70 and GITR-L.

REFERENCES

1. Vinay et al. 1998, Sem. Immunol. 10:481.

2. Watts et al. 1999, Curr. Op. Immunol. 11:286.

3. Goodwin et al. 1993, Eur. J. Immunol. 23:2631.

4. Pollok et al. 1994, Eur. J. Immunol. 24:367.

5. DeBenedette et al. 1997, J. Immunol. 158:551.

6. Saoulli et al. 1998, J. Exp. Med. 187:1849.

7. Shuford et al. 1997, J. Exp. Med. 186:47.

8. Takahashi et al. 1999, J. Immunol. 162:5037.

9. DeBenedette et al. 1995, J. Exp. Med. 181:985.

10. Chu et al. 1997, J. Immunol. 158:3081.

11. Gramaglia et al. 2000, Eur. J. inmunol. 30:392.

12. Blazar et al. 2001, J Immunol 166:3174.

13. Cannons et al. 2001, J. Immunol. 167:1313.

14. DeBenedette et al. 1999, J. Tmmunol. 163:4833.

15. Tan et al. 1999, J. Immunol. 163:4859.

16. Tan et al. 2000, J. Immunol. 164:2320.

17. Wang et al. 1998, AIDS Res. Hum. Retro. 14:223.

18. Melero et al. 1997, Nature Med. 3:682.

19. Melero et al. 1998, Eur. J. Immunol. 28:1116.

20. Guinn et al. 1999, J. Immunol. 162:5003.

21. Chen et al. 2000, Mol Ther 2:39.

22. Alderson et al. 1994, Eur. J. Immunol. 24:2219.

23. Zhou et al. 1995, Immunology Letters 45:67.

24. Schwarz et al. 1995, Blood 85:1043.

25. Kimm et al. 1993, J. Immunol. 151:1255.

26. Arch et al. 1998, Mol.Cell Biol. 18:558.

27. Jang et al. 1998, Bioch. Biophys. Res. Comm. 242:613.

28. Ye et al. 1999, Mol. Cell. 4:321.

29. Cannons et al. 1999, J. Immunol. 163:2990.

30. Cannons et al. 2000, J Immunol. 165:6193.

31. Broll et al. 2001, Am J Clin Pathol 115:543.

32. Schwarz,et al. 1997, Biophys. Res. Comm. 235:699.

33. Michel et al. 1998, Eur. J. Immunol 28:290.

34. Kienzle et al. 2000, int Immunol 12:73.

35. Heinisch et al. 2000, Eur. J. Immunol 30:3441.

36. Schwarz et al. 1996, Blood 87:2839.

37. Kim et al. 1998, Eur. J. Immunol. 28:881.

38. Langstein et al. 1999, J. Leukoc Biol 65:829.

39. Langstein et al. 1999, Blood 94:3161.

40. Dustin et al. 1999, Science 283:649-650.

41. Lenschow et al. 1996, Ann. Rev. Immunol. 14:233.

42. McAdam et al. 1998, Immunological Reviews 165:231-47.

43. Azuma et al. 1993, J. Immunol 150:1147-59.

44. Effros et al. 1994, Exp. Gerontol 29:601-9.

45. Mugnaini et al. 1998, Eur. J. Immunol 28:1738-42.

46. Scott-Algara et al. 2001, J. Infect Dis 183:1565-73.

47. Trimble et al. 2000, J. Virol 74:7320-30.

48. Weekes et al. 1999, J. Immunol 162:7569-77.

49. Speiser et 1999, Eur. J. Immunol 29:1990-9.

50. Monteiro et al. 1996, J. Immunol 156:3587-90.

51. Park et al. 1997, Eur. J. Immunol 27:1082-90.

52. Schmidt et al. 1996, J. Clin Invest 97:2027-37.

53. Sze et 2001, Blood 98:2817-27.

54. Pollok et al. 1993, J. Immunol 150:771-781.

55. Kwon et al. 2000, Molecules and Cells 10:119-26.

56. Bertram et al. 2002, J. Immunol. 168:3777.

57. Maus et al. 2002, Nature Biotechnology 20:143-8.

58. Wen et al. 2002, J. Immunol. 168:4897.

59. Nociari et al. 1999, J. Immunol. 162:3327-35.

60. Boise et al. 1995, Immunity 3, 87-98.

61. Brinkmann et al. 1994, J. Infec. Dis. 169:730-738.

62. Martens et al. 1997, Arthritis Rheumatism 40:1106-14.

63. Honda et al. 2001, Clin. Immunol. 99:211-221.

64. Markovic-Plese et al. 2001, J. Clin. Invest. 108:1185-94.

65. Hislop et al. 2001, J. Immunol. 167:2019-29.

66. Swigut et al. 2001, Embo J 20:1593-604.

67. Hafler et al. 1989, Immunology Today 10:204.

68. Shahinian et al. 1993, Science 261:609.

69. June 2002, Nature Biotechnology 20:143.

70. Levineet al.2002, Nat. Med. 8:47.

71. Schuleret al. 2003, Curr Opin Immunol 15:138.

72. Steinmanet al. 2002, J. Clin. Invest. 109:1519.

73. Steinmanet al. 2001, Int J Cancer 94:459.

74. Sharpe et al. 2002, Nat. Rev. Immunol. 2:116.

75. Croft 2003, Nat. Rev. Immunol. 3:609.

76. Xiang 1999, Cancer Biother. Radiopharm. 14:353.

77. Martinetet al. 2000, J. Natl. Cancer Inst. 92:931.

78. Stromeet al. 2000, J. Immunother. 23:430.

79. Ye et al.2002, Nat. Med. 8:343.

80. Laderachet al. 2002, Int. Immunol. 14:1155.

81. Miller et al. 2002, J. Immunol. 169:1792.

82. Yoshida et al. 2003, Cancer Immunol. Immunother. 52:97.

83. Croft 2003, Cytokine Growth Factor Rev. 14:265.

84. Futagawa et al. 2002, Int. Immunol. 14:275.

85. Hurtado et al. 1995, J. Immunol. 155:3360.

86. Hurtado et al. 1997, J. Immunol. 158:2600.

87. Cooper et al. 2002, Eur. J. Immunol. 32:521.

88. Diehl et al. 2002, J. Immunol. 168:3755.

89. Wilcox et al. 2002, J. Clin. Invest. 109:651.

90. Wiethe et al. 2003, J. Immunol. 170:2912.

91. Melero et al. 1998, Cell. Immunol. 190:167.

92. Halstead et al. 2002, Nat. Immunol. 3:536.

93. Bukczynski et al. 2003, Eur. J. Immunol. 33:446.

94. Kim et al. 2002, Blood 100:3253.

95. Appay et al. 2002, Nature Med. 8:379.

96. Bansal-Pakala et al. 2002, J. Immunol. 169:5005.

97. Altman et al. 1996, Science 274:94.

98. Bett et al. 1994, Proc. Natl. Acad. Sci. U.S.A. 91:8802.

99. Harlin et al. 2002, J. Immunol. 169:2451.

100. Tamada et al. 2002, J. Immunol. 168:4832.

101. Scheu et al. 2002, J. Exp. Med. 195:1613.

102. Granger et al. 2003, Cytokine Growth Factor Rev. 14:289.

103. Weinberg et al. 1998, Sem. Immunol. 10:471.

104. Walker et al. 2000, Immunol. Today 21:333.

105. Gramaglia et al. 1998, J. Immunol. 161:6510.

106. Gramaglia et al. 2000, J. Immunol. 165:3043.

107. Rogers et al. 2001, Immunity 15:445.

108. Maxwell et al. 2000, J. Immunol. 164:107.

109. De Smedt et al. 2002, J. Immunol. 168:661.

110. Mauri et al. 1998, Immunity 8:21.

111. Liu et al. 2003, Int Immunol 15:861.

112. Ye et al. 2002, J. Exp. Med. 195:795.

What is claimed is:

1. A method of preparing an antigen presenting cell specific to an antigen, said method comprising:
(a) providing a human monocyte or monocyte-related cell;
(b) increasing the level of a 4-1BBL in said monocyte or monocyte-related cell;
(c) increasing the level of a B7 molecule in said monocyte or monocyte-related cell;
(d) contacting said monocyte or monocyte-related cell with said antigen or a part thereof; and
(e) culturing said monocyte or monocyte-related cell for less than 1 week thereby to allow its conversion to an antigen presenting cell.

2. The method of claim 1, wherein said level of a 4-1BBL is increased by introducing into said monocyte or monocyte-related cell a nucleic acid encoding said 4-1BBL.

3. The method of claim 2, wherein said nucleic acid is introduced into said monocyte by introducing into said monocyte a vector comprising said nucleic acid.

4. The method of claim 3, wherein said vector is a viral vector.

5. The method of claim 4, wherein said vector is an adenovirus.

6. The method of claim 1, wherein said level of a B7 molecule is increased by introducing into said monocyte or monocyte-related cell a nucleic acid encoding said B7 molecule.

7. The method of claim 6, wherein said nucleic acid is introduced into said monocyte by introducing into said monocyte a vector comprising said nucleic acid.

8. The method of claim 7, wherein said vector is a viral vector.

9. The method of claim 8, wherein said vector is an adenovirus.

10. The method of claim 2, wherein said level of a B7 molecule is increased by introducing into said monocyte or monocyte-related cell a nucleic acid encoding said B7 molecule.

11. The method of claim 10, wherein said nucleic acid encoding said 4-1BBL is introduced into said monocyte or monocyte-related cell by introducing into said monocyte or monocyte-related cell a vector comprising said nucleic acid encoding said 4-1BBL, and wherein said nucleic acid encoding said B7 molecule is introduced into said monocyte or monocyte-related cell by introducing into said monocyte or monocyte-related cell a vector comprising said nucleic acid encoding said B7 molecule.

12. The method of claim 11, wherein said vector comprising said nucleic acid encoding said 41BBL and said vector comprising said nucleic acid encoding said B7 molecule are each independently a viral vector.

13. The method of claim 4, wherein said viral vector is an adenovirus.

14. The method of claim 1, wherein said method further comprises contacting said T cell with a TNF ligand.

15. The method of claim 14, wherein said TNF ligand is selected from the group consisting of OX40L, LIGHT, CD70, CD30 and GITR-L.

16. The method of claim 1, wherein said culturing step (e) has a duration of less than about 72 hours.

17. The method of claim 16, wherein said culturing step (e) has a duration of less than about 48 hours.

18. The method of claim 17, wherein said culturing step (e) has a duration of less than about 24 hours.

19. The method of claim 18, wherein said culturing step (e) has a duration of less than about 16 hours.

20. The method of claim 19, wherein said culturing step (e) has a duration of about 12 to about 16 hours.

21. The method of claim 1, wherein said antigen or part thereof is selected from a virus, a protein and a polypeptide.

22. The method of claim 21, wherein said protein or polypeptide is contacted with said monocyte or monocyte-related cell by introducing into said monocyte or monocyte-related cell a nucleic acid capable of encoding said protein or said polypeptide.

23. An antigen presenting cell produced-by the method of claim 10, wherein said antigen presenting cell comprises:
(a) a recombinant vector comprising said nucleic acid encoding said 41BBL; and
(b) a recombinant vector comprising said nucleic acid encoding said B7 molecule.

24. The antigen presenting cell of claim 24, wherein the same vector comprises both said nucleic acid encoding said 41BBL and said nucleic acid encoding said B7 molecule.

25. A composition comprising the antigen presenting cell of claim 23 and a pharmaceutically acceptable carrier.

26. A vaccine comprising the antigen presenting cell of claim 23.

27. A method of activating a human T cell, said method comprising contacting said T cell with the antigen presenting cell of claim 23.

28. The method of claim 27, wherein said contacting step is carried out ex vivo.

29. The method of claim 27, wherein said contacting step is carried out in vivo and said antigen presenting cell is administered to a subject comprising said human T cell.

30. The method of claim 27, wherein said human T cell is obtained from a subject suffering from a condition associated with immuno impairment.

31. The method of claim 30, wherein said condition is selected from the group consisting of viral disease, pathogen infection and cancer.

32. The method of claim 31, wherein said viral disease is selected from the group consisting of AIDS, hepatitis C, and CMV-related disease.

33. The method of claim 31, wherein said pathogen is selected from the group consisting or a bacteria, a fungus and a parasite.

34. A method of treating a subject suffering from a condition associated with immuno impairment, said method comprising administering to said subject the antigen presenting cell of claim 23.

35. A method of vaccinating a subject, said method comprising administering to said subject the antigen presenting cell of claim 23.

36. A method of treating a subject suffering from a condition associated with immuno impairment, said method comprising administering to said subject an activated T cell of claim prepared by the method of claim 27.

37. The method of claim 1, wherein said B7 molecule is selected from the group consisting of B7.1 and B7.2.


SEQUENCE LISTING (11 sequences)

SEQ ID NO: 1 - DNA, 1619 bp, Homo sapiens
  CDS (4)..(768)
gtcatggaatacgcctctgacgcttcactggaccccgaagccccgtggcctcccgcgccccgcgctcgcgcctgccgcgtactgccttgggccctggtcgcggggctgctgctgctgctgctgctcgctgccgcctgcgccgtcttcctcgcctgcccctgggccgtgtccggggctcgcgcctcgcccggctccgcggccagcccgagactccgcgagggtcccgagctttcgcccgacgatcccgccggcctcttggacctgcggcagggcatgtttgcgcagctggtggcccaaaatgttctgctgatcgatgggcccctgagctggtacagtgacccaggcctggcaggcgtgtccctgacggggggcctgagctacaaagaggacacgaaggagctggtggtggccaaggctggagtctactatgtcttctttcaactagagctgcggcgcgtggtggccggcgagggctcaggctccgtttcacttgcgctgcacctgcagccactgcgctctgctgctggggccgccgccctggctttgaccgtggacctgccacccgcctcctccgaggctcggaactcggccttcggtttccagggccgcttgctgcacctgagtgccggccagcgcctgggcgtccatcttcacactgaggccagggcacgccatgcctggcagcttacccagggcgccacagtcttgggactcttccgggtgacccccgaaatcccagccggactcccttcaccgaggtcggaataacgcccagcctgggtgcagcccacctggacagagtccgaatcctactccatccttcatggagacccctggtgctgggtccctgctgctttctctacctcaaggggcttggcaggggtccctgctgctgacctccccttgaggaccctcctcacccactccttccccaagttggaccttgatatttattctgagcctgagctcagataatatattatatatattatatatatatatatatttctatttaaagaggatcctgagtttgtgaatggacttttttagaggagttgttttggggggggggtcttcgacattgccgaggctggtcttgaactcctggacttagacgatcctcctgcctcagcctcccaagcaactgggattcatcctttctattaattcattgtacttatttgcctatttgtgtgtattgagcatctgtaatgtgccagcattgtgcccaggctagggggctatagaaacatctagaaatagactgaaagaaaatctgagttatggtaatacgtgaggaatttaaagactcatccccagcctccacctcctgtgtgatacttgggggctagcttttttctttctttcttttttttgagatggtcttgttctgtcaaccaggctagaatgcagcggtgcaatcatgagtcaatgcagcctccagcctcgacctcccgaggctcaggtgatcctcccatctcagcctctcgagtagctgggaccacagttgtgtgccaccacacttggctaactttttaatttttttgcggagacggtattgctatgttgccaaggttgtttacatgccagtacaatttataataaacactcatttttcc

SEQ ID NO: 2 - protein, 254 aa, Homo sapiens
MEYASDASLDPEAPWPPAPRARACRVLPWALVAGLLLLLLLAAACAVFLACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 3 - DNA, 2824 bp, Homo sapiens
  CDS (364)..(1230)
aagtaacagaagttagaaggggaaatgtcgcctctctgaagattacccaaagaaaaagtgatttgtcattgctttatagactgtaagaagagaacatctcagaagtggagtcttaccctgaaatcaaaggatttaaagaaaaagtggaatttttcttcagcaagctgtgaaactaaatccacaacctttggagacccaggaacaccctccaatctctgtgtgttttgtaaacatcactggagggtcttctacgtgagcaattggattgtcatcagccctgcctgttttgcacctgggaagtgccctggtcttacttgggtccaaattgttggctttcacttttgaccctaagcatctgaagccatgggccacacacggaggcagggaacatcaccatccaagtgtccatacctcaatttctttcagctcttggtgctggctggtctttctcacttctgttcaggtgttatccacgtgaccaaggaagtgaaagaagtggcaacgctgtcctgtggtcacaatgtttctgttgaagagctggcacaaactcgcatctactggcaaaaggagaagaaaatggtgctgactatgatgtctggggacatgaatatatggcccgagtacaagaaccggaccatctttgatatcactaataacctctccattgtgatcctggctctgcgcccatctgacgagggcacatacgagtgtgttgttctgaagtatgaaaaagacgctttcaagcgggaacacctggctgaagtgacgttatcagtcaaagctgacttccctacacctagtatatctgactttgaaattccaacttctaatattagaaggataatttgctcaacctctggaggttttccagagcctcacctctcctggttggaaaatggagaagaattaaatgccatcaacacaacagtttcccaagatcctgaaactgagctctatgctgttagcagcaaactggatttcaatatgacaaccaaccacagcttcatgtgtctcatcaagtatggacatttaagagtgaatcagaccttcaactggaatacaaccaagcaagagcattttcctgataacctgctcccatcctgggccattaccttaatctcagtaaatggaatttttgtgatatgctgcctgacctactgctttgccccaagatgcagagagagaaggaggaatgagagattgagaagggaaagtgtacgccctgtataacagtgtccgcagaagcaaggggctgaaaagatctgaaggtctcacctccatttgcaattgacctcttctgggaacttcctcagatggacaagattaccccaccttgccctttacgtatctgctcttaggtgcttcttcacttcagttgctttgcaggaagtgtctagaggaatatggtgggcacagaagtagctctggtgaccttgatcaaggggttttgaaatgcagaattcttgagttctggaagggactttagagaataccagtgttattaatgacaaaggcactgaggcccagggaggtgacccgaattataaaggccagcgccagaacccagatttcctaactctggtgctctttccctttatcagtttgactgtggcctgttaactggtatatacatatatatgtcaggcaaagtgctgctggaagtagaatttgtccaataacaggtcaacttcagagactatctgatttcctaatgtcagagtagaagattttatgctgctgtttacaaaagcccaatgtaatgcataggaagtatggcatgaacatctttaggagactaatggaaatattattggtgtttacccagtattccatttttttcattgtgttctctattgctgctctctcactcccccatgaggtacagcagaaaggagaactatccaaaactaatttcctctgacatgtaagacgaatgatttaggtacgtcaaagcagtagtcaaggaggaaagggatagtccaaagacttaactggttcatattggactgataatctctttaaatggctttatgctagtttgacctcatttgtaaaatatttatgagaaagttctcatttaaaatgagatcgttgtttacagtgtatgtactaagcagtaagctatcttcaaatgtctaaggtagtaactttccatagggcctccttagatccctaagatggctttttctccttggtatttctgggtctttctgacatcagcagagaactggaaagacatagccaactgctgttcatgttactcatgactcctttctctaaaactgccttccacaattcactagaccagaagtggacgcaacttaagctgggataatcacattatcatctgaaaatctggagttgaacagcaaaagaagacaacatttctcaaatgcacatctcatggcagctaagccacatggctgggatttaaagcctttagagccagcccatggctttagctacctcactatgctgcttcacaaaccttgctcctgtgtaaaactatattctcagtgtagggcagagaggtctaacaccaacataaggtactagcagtgtttcccgtattgacaggaatacttaactcaataattcttttcttttccatttagtaacagttgtgatgactatgtttctattctaagtaattcctgtattctacagcagatactttgtcagcaatactaagggaagaaacaaagttgaaccgtttctttaataaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 4 - protein, 288 aa, Homo sapiens
MGHTRRQGTSPSKCPYLNFFQLLVLAGLSHFCSGVIHVTKEVKEVATLSCGHNVSVEELAQTRIYWQKEKKMVLTMMSGDMNIWPEYKNRTIFDITNNLSIVILALRPSDEGTYECVVLKYEKDAFKREHLAEVTLSVKADFPTPSISDFEIPTSNIRRIICSTSGGFPEPHLSWLENGEELNAINTTVSQDPETELYAVSSKLDFNMTTNHSFMCLIKYGHLRVNQTFNWNTTKQEHFPDNLLPSWAITLISVNGIFVICCLTYCFAPRCRERRRNERLRRESVRPV

SEQ ID NO: 5 - DNA, 2781 bp, Homo sapiens
  CDS (117)..(1106)
ggaaggcttgcacagggtgaaagctttgcttctctgctgctgtaacagggactagcacagacacacggatgagtggggtcatttccagatattaggtcacagcagaagcagccaaaatggatccccagtgcactatgggactgagtaacattctctttgtgatggccttcctgctctctggtgctgctcctctgaagattcaagcttatttcaatgagactgcagacctgccatgccaatttgcaaactctcaaaaccaaagcctgagtgagctagtagtattttggcaggaccaggaaaacttggttctgaatgaggtatacttaggcaaagagaaatttgacagtgttcattccaagtatatgggccgcacaagttttgattcggacagttggaccctgagacttcacaatcttcagatcaaggacaagggcttgtatcaatgtatcatccatcacaaaaagcccacaggaatgattcgcatccaccagatgaattctgaactgtcagtgcttgctaacttcagtcaacctgaaatagtaccaatttctaatataacagaaaatgtgtacataaatttgacctgctcatctatacacggttacccagaacctaagaagatgagtgttttgctaagaaccaagaattcaactatcgagtatgatggtattatgcagaaatctcaagataatgtcacagaactgtacgacgtttccatcagcttgtctgtttcattccctgatgttacgagcaatatgaccatcttctgtattctggaaactgacaagacgcggcttttatcttcacctttctctatagagcttgaggaccctcagcctcccccagaccacattccttggattacagctgtacttccaacagttattatatgtgtgatggttttctgtctaattctatggaaatggaagaagaagaagcggcctcgcaactcttataaatgtggaaccaacacaatggagagggaagagagtgaacagaccaagaaaagagaaaaaatccatatacctgaaagatctgatgaagcccagcgtgtttttaaaagttcgaagacatcttcatgcgacaaaagtgatacatgtttttaattaaagagtaaagcccatacaagtattcattttttctaccctttcctttgtaagttcctgggcaacctttttgatttcttccagaaggcaaaaagacattaccatgagtaataagggggctccaggactccctctaagtggaatagcctccctgtaactccagctctgctccgtatgccaagaggagactttaattctcttactgcttcttttcacttcagagcacacttatgggccaagcccagcttaatggctcatgacctggaaataaaatttaggaccaatacctcctccagatcagattcttctcttaatttcatagattgtgtttttttttaaatagacctctcaatttctggaaaactgccttttatctgcccagaattctaagctggtgccccactgaatcttgtgtacctgtgactaaacaactacctcctcagtctgggtgggacttatgtatttatgaccttatagtgttaatatcttgaaacatagagatctatgtactgtaatagtgtgattactatgctctagagaaaagtctacccctgctaaggagttctcatccctctgtcagggtcagtaaggaaaacggtggcctagggtacaggcaacaatgagcagaccaacctaaatttggggaaattaggagaggcagagatagaacctggagccacttctatctgggctgttgctaatattgaggaggcttgccccacccaacaagccatagtggagagaactgaataaacaggaaaatgccagagcttgtgaaccctgtttctcttgaagaactgactagtgagatggcctggggaagctgtgaaagaaccaaaagagatcacaatactcaaaagagagagagagagaaaaaagagagatcttgatccacagaaatacatgaaatgtctggtctgtccaccccatcaacaagtcttgaaacaagcaacagatggatagtctgtccaaatggacataagacagacagcagtttccctggtggtcagggaggggttttggtgatacccaagttattgggatgtcatcttcctggaagcagagctggggagggagagccatcaccttgataatgggatgaatggaaggaggcttaggactttccactcctggctgagagaggaagagctgcaacggaattaggaagaccaagacacagatcacccggggcttacttagcctacagatgtcctacgggaacgtgggctggcccagcatagggctagcaaatttgagttggatgattgtttttgctcaaggcaaccagaggaaacttgcatacagagacagatatactgggagaaatgactttgaaaacctggctctaaggtgggatcactaagggatggggcagtctctgcccaaacataaagagaactctggggagcctgagccacaaaaatgttcctttattttatgtaaaccctcaagggttatagactgccatgctagacaagcttgtccatgtaatattcccatgtttttaccctgcccctgccttgattagactcctagcacctggctagtttctaacatgttttgtgcagcacagtttttaataaatgcttgttacattcaaaaaaaaaaaaaaaaaaaaaaaaaaaaaaaaaaaaaaaaaaaaaaaaaaaaaaaaaaaaaaaaaaaaaaaaaaaaa

SEQ ID NO: 6 - protein, 329 aa, Homo sapiens
MDPQCTMGLSNILFVMAFLLSGAAPLKIQAYFNETADLPCQFANSQNQSLSELVVFWQDQENLVLNEVYLGKEKFDSVHSKYMGRTSFDSDSWTLRLHNLQIKDKGLYQCIIHHKKPTGMIRIHQMNSELSVLANFSQPEIVPISNITENVYINLTCSSIHGYPEPKKMSVLLRTKNSTIEYDGIMQKSQDNVTELYDVSISLSVSFPDVTSNMTIFCILETDKTRLLSSPFSIELEDPQPPPDHIPWITAVLPTVIICVMVFCLILWKWKKKKRPRNSYKCGTNTMEREESEQTKKREKIHIPERSDEAQRVFKSSKTSSCDKSDTCF

SEQ ID NO: 7 - protein, 8 aa, Influenza virus
  misc_feature: M1 amino acid sequence
GILGVFTL

SEQ ID NO: 8 - protein, 9 aa, Epstein-Barr virus
  misc_feature: BMLF1 sequence
GLCTLVAML

SEQ ID NO: 9 - protein, 9 aa, Human immunodeficiency virus
  misc_feature: gag 77-85 peptide
SLYNTVATL

SEQ ID NO: 10 - protein, 8 aa, Human immunodeficiency virus
  misc_feature: nef 88-95 peptide
FLKEKGGL

SEQ ID NO: 11 - protein, 9 aa, Homo sapiens
  misc_feature: melanoma gp100 peptide
IMDQVPFSV